(19) United States
(12) Patent Application Publication
(10) Pub. No.: US 2026/0151517 A1
(43) Pub. Date: Jun. 4, 2026

(54) SOMATOSTATIN RECEPTOR-TARGETING RADIOPHARMACEUTICALS AND USES THEREOF

(71) Applicants: Nuclidium AG, Basel (CH); University of Basel, Basel (CH)

(72) Inventors: Leila Jaafar-Thiel, Basel (CH); Francesco De Rose, Basel (CH); Melpomeni Fani, Basel (CH)

(21) Appl. No.: 19/375,103

(22) Filed: Oct. 30, 2025

Related U.S. Application Data
(63) Continuation of application No. PCT/US2025/021199, filed on Mar. 24, 2025.
(60) Provisional application No. 63/568,814, filed on Mar. 22, 2024.

(51) Int. Cl. A61K 51/08; A61K 47/02; A61K 47/10; A61K 47/22

(52) U.S. Cl. CPC A61K 51/083; A61K 47/02; A61K 47/10; A61K 47/22; A61K 51/088; A61K 2123/00

(57) ABSTRACT

The present disclosure relates to pharmaceutical compositions comprising [⁶¹Cu]Cu-NODAGA-LM3 or a pharmaceutically acceptable salt thereof. The present disclosure also relates to methods of imaging subjects suspected of having or diagnosed with a somatostatin receptor subtype 2 (SST2)-expressing tumor by administering the pharmaceutical compositions described herein and generating one or more radiographic images of the subject.

### 1. BACKGROUND

Neuroendocrine tumors (NET) are a group of neoplasms arising from neuroendocrine cells and are most commonly found in the gastrointestinal tract, pancreas, and lungs. Although NETs are rare, their incidence rates have been significantly increasing. As the majority of NETs are slowly growing tumors with almost no symptoms, up to 50% are metastatic at diagnosis.

The somatostatin receptor subtype 2 (SSTR2) is a heterodimeric G protein-coupled transmembrane receptor highly expressed in NETs. SSTR2 expression constitutes the pathophysiological basis for diagnosing NETs using nuclear medicine in vivo techniques such as positron emission tomography (PET).

In one clinical study, the diagnostic efficiency of detecting NETs with PET/CT imaging using [⁶⁸Ga]Ga-NODAGA-LM3 (a radiolabeled SSTR2 antagonist) and [⁶⁸Ga]Ga-DOTA-TATE (an FDA-approved radiolabeled SSTR2 agonist) was compared. [⁶⁸Ga]Ga-NODAGA-LM3 had lower uptake in normal organs and higher tumor-to-background ratios than [⁶⁸Ga]Ga-DOTA-TATE. [⁶⁸Ga]Ga-NODAGA-LM3 had also higher tumor uptake and detected more lesions than [⁶⁸Ga]Ga-DOTA-TATE (Zhu W, et al., EJNMMI 2022:49:1613-1622). In a single-center, prospective phase I/II head-to-head comparative study with 12 well-differentiated gastroenteropancreatic (GEP) NET patients, [⁶⁸Ga]Ga-NODAGA-JR11 (a radiolabeled SST antagonist), also known as [⁶⁸Ga]Ga-OPS202, showed a significantly higher sensitivity, on a lesion-basis, than [⁶⁸Ga]Ga-DOTATOC (Nicolas G P, et al., J Nucl Med. 2018; 59 (6): 915-21).

Use of [⁶⁸Ga]Ga-labelled imaging agents is hampered by (i) the expense and limited production capacity of 68Ge/⁶⁸Ga-generators, (ii) the short half-life of ⁶⁸Ga (t1/2=1.13 h), which prevents the shipment of the radiotracer from central producers to smaller centers that are located beyond 2 hours' distance, and (iii) the high-energy of ⁶⁸Ga (Emax=1.9 MeV), which limits spatial resolution of reconstructed PET imaging.

There is a need for improved methods of generating radiographic images of subjects with SSTR2-expressing tumors for the diagnosis, monitoring, and ultimately, treatment of cancer patients.

### 2. SUMMARY

In one aspect, the present disclosure provides a pharmaceutical composition comprising:
(a) a radiotracer that has the structure

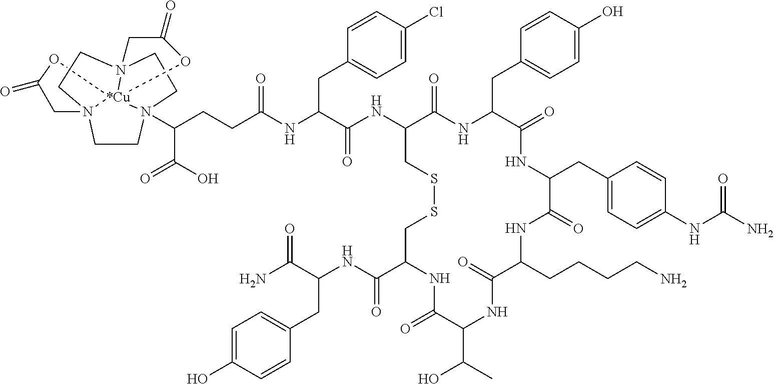

or is a pharmaceutically acceptable salt thereof, wherein,
(b) a radiolytic inhibitor, such as ascorbic acid, and
(c) an aqueous vehicle;
   wherein the pharmaceutical composition is characterized by one or more of:
a [*Cu]Cu, particularly [⁶¹Cu]Cu, radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a ¹⁰⁹Cd specific activity≤0.1 Bq/g.

In another aspect, the present disclosure provides a method for imaging a subject comprising:
(a) administering an effective amount of a pharmaceutical composition to a subject suspected of having or diagnosed with a somatostatin receptor subtype 2 (SST2)-expressing tumor, wherein the pharmaceutical composition comprises:
   i. a radiotracer that has the structure

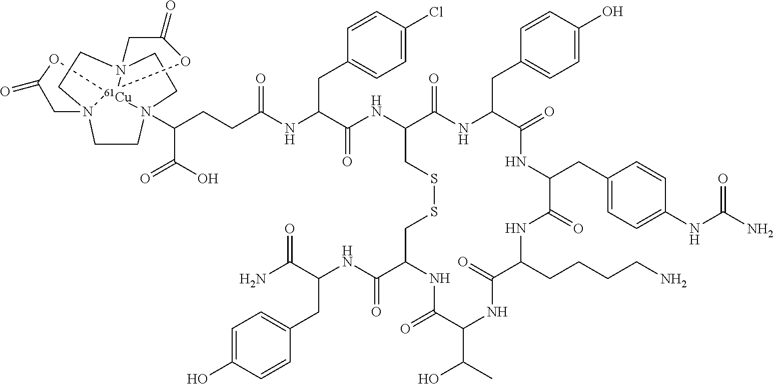

or is a pharmaceutically acceptable salt thereof, and
   ii, a radiolytic inhibitor, such as ascorbic acid;
wherein the pharmaceutical composition is characterized by one or more of:
   a [⁶¹Cu]Cu radionuclidic purity at end of synthesis of ≥97%,
   a radiocobalt activity content at end of synthesis of ≤0.05%,
   a ^{110m}Ag specific activity≤0.1 Bq/g,
   a ^{108m}Ag specific activity≤0.1 Bq/g, or
   a ¹⁰⁹Cd specific activity≤0.1 Bq/g; and
(b) generating one or more radiographic images of the subject.

In another aspect, the present disclosure provides a method for determining a subject's response to a cancer treatment comprising:
(a) administering an effective amount of a pharmaceutical composition to a subject diagnosed with a SST2-expressing tumor at an earlier time point and at a later time point, wherein the pharmaceutical composition comprises:
   i. a radiotracer that has the structure

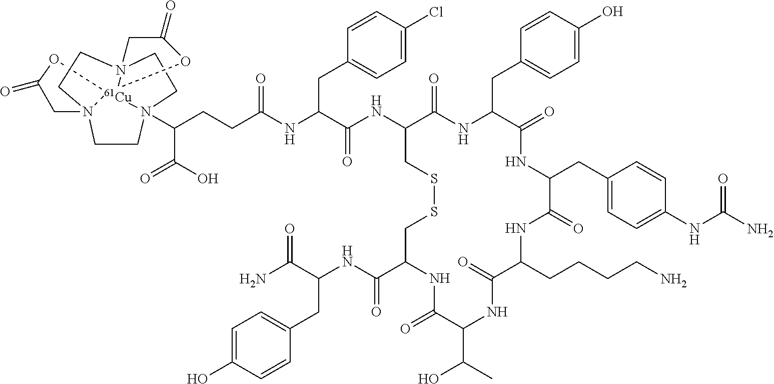

or is a pharmaceutically acceptable salt thereof, and
   ii, a radiolytic inhibitor, such as ascorbic acid;
wherein the pharmaceutical composition is characterized by one or more of:
   a [⁶¹Cu]Cu radionuclidic purity at end of synthesis of ≥97%,
   a radiocobalt activity content at end of synthesis of ≤0.05%,
   a ^{110m}Ag specific activity≤0.1 Bq/g,
   a ^{108m}Ag specific activity≤0.1 Bq/g, or
   a ¹⁰⁹Cd specific activity≤0.1 Bq/g;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

In yet another aspect, the present disclosure provides a theranostic method comprising:
(a) administering to a subject an effective amount of a first pharmaceutical composition described herein;
(b) generating one or more images of the subject; and
(c) administering to the subject an effective amount of a second pharmaceutical composition comprising a radiopharmaceutical.

### 3. BRIEF DESCRIPTION OF THE DRAWINGS

These and other features, aspects, and advantages of the present disclosure will become better understood with regard to the following description, and accompanying drawings, where:

FIG. 1 illustrates with increasing magnification homogenous nickel coating having durable adhesion to a niobium coin upon completion of electroplating, as evaluated using a DINOLite digital microscope. Panel A. 20× magnification; panel B, 50× magnification; panel C, 250× magnification.

FIG. 2 shows samples of the coin provided according to the present disclosure with nickel deposited in the center of a niobium backing.

FIG. 3 displays the analysis of ⁶¹Cu purity of [⁶¹Cu]CuCl₂ solution obtained by irradiation of ^{nat}Ni on Nb backing with deuteron beam at 8.4 MeV for 3 h at 50 μA. The curved line corresponds to reduction in % purity of ⁶¹Cu over time and the bars correspond to radiocobalt activity over time.

FIG. 4 displays an analysis of ⁶¹Cu purity of [⁶¹Cu]CuCl₂ solution obtained by irradiation of ⁶⁰Ni on Nb backing with a deuteron beam at 8.4 MeV for 3 h at 50 μA. The curved line corresponds to the reduction in % purity of ⁶¹Cu over time, and the bars correspond to radiocobalt activity over time.

FIG. 5 presents the specific activity of detected impurities in [⁶¹Cu]CuCl₂ solutions produced according to various methods. The ext. coin (Ag, natNi) data was generated by irradiation of a commercially available ^{nat}Ni target on Ag backing. The (Nb, natNi) and (Nb, Ni-61) data were generated based on irradiation of Ni targets (natural and isotopically enriched in ⁶¹Ni, respectively) electroplated according to the present disclosure on high-purity Nb backing. The specific activity was assessed by gamma spectrometry and reported in Bq/g. The data shows that silver and cobalt isotopes are significantly reduced in the [⁶¹Cu]CuCl₂ solution produced by irradiation of Ni targets electroplated according to the present disclosure on high-purity Nb backing.

FIG. 6 shows the significant reduction in the sum of radionuclidic impurities present in a [⁶¹Cu]CuCl₂ solutions produced according to various methods. The ext. coin (Ag, natNi) data was generated based on irradiation of a commercially available ^{nat}Ni target on Ag backing. The (Nb, natNi) and (Nb, Ni-61) data were generated based on irradiation of Ni targets (natural and isotopically enriched in ⁶¹Ni, respectively), electroplated according to the present disclosure on high-purity Nb backing. The radionuclidic impurities were determined by gamma spectrometry and reported in Bq/g (summed radionuclidic impurities). The presented data highlight in particular the reduction of overall impurities in the [⁶¹Cu]CuCl₂ solution when produced in accordance with the present disclosure.

FIG. 7 illustrates the sustained high radionuclidic purity of a [⁶¹Cu]CuCl₂ solution produced according to the present disclosure compared to a commercially available ^{nat}Ni target on a Ag backing (ext. coin (Ag, natNi)). The (Nb, natNi) and (Nb, Ni-61) coins were prepared by electrodeposition according to the present disclosure on high-purity Nb backing. The data was generated using gamma spectrometry and reported in Bq/g providing the summed radionuclidic purities at t=0 h and at t=12 h. The presented data highlight the superior quality of the [⁶¹Cu]CuCl₂ solution when produced by irradiation of Ni targets electroplated according to the present disclosure on high purity Nb backing, where the purity after 12 hours is still well above the purity limits set by pharmacopeia for similar radionuclides for medical use.

FIG. 8 displays chemical impurities, as measured by ICP-MS, of the [⁶¹Cu]CuCl₂ solution when produced by bombardment of ^{nat}Ni vs. ⁶¹Ni when produced by irradiation of Ni targets electroplated according to the present disclosure on high-purity Nb backing.

FIG. 9 shows the process for automated cassette production of [⁶¹Cu]Cu-NODAGA-LM3.

FIG. 10 displays maximum intensity projections (MIPs) PET/CT images of [⁶¹Cu]Cu-NODAGA-LM3 at 1 hour (dynamic scan) and 4 hours post-injection in HEK-SST2 xenografted mice. Scale 0-4 in standardized uptake value (SUV).

FIG. 11 illustrates the study protocol described in Example 4.

FIG. 12 details the study procedures and assessments as described in Example 4.

FIG. 13 shows [⁶¹Cu]Cu-NODAGA-LM3 PET/CT and [⁶⁸Ga]Ga-DOTATOC PET/CT of Subject 1.

FIG. 14 shows [⁶¹Cu]Cu-NODAGA-LM3 PET/CT and [⁶⁸Ga]Ga-DOTATOC PET/CT of Subject 2.

FIG. 15 shows [⁶¹Cu]Cu-NODAGA-LM3 PET/CT and [⁶⁸Ga]Ga-DOTATOC PET/CT of Subject 3.

FIG. 16 shows [⁶¹Cu]Cu-NODAGA-LM3 PET/CT of Subject 4.

FIG. 17 shows [⁶¹Cu]Cu-NODAGA-LM3 PET/CT of Subject 6.

FIG. 18 shows AMA test plot obtained plotting at x-axis (logarithmic) the nmol of titrating chelator and at y-axis the % of complexation obtained via radio-TLC.

FIG. 19 shows experimental point corresponding to the lowest value of nmol of chelator in correspondence of which ≥95% complexation was achieved (black arrow).

### 4. DETAILED DESCRIPTION

### 4.1. Definitions

When describing the embodiments of the present disclosure, the following terms, if present, have the following meanings, unless otherwise indicated. If not otherwise defined, terms have their customary meaning in the relevant art.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least.” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than.” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

As used herein, “absorbed dose” refers to the amount of radiation absorbed by an object (e.g., an organ or tissue) or a person. The gray (Gy) is the SI unit of absorbed dose and is defined as the absorption of one joule of energy, in the form of ionizing radiation, per kilogram of matter, i.e., one gray=1 J/kg².

As used herein, “activity” or “radioactivity” refers to a physical quantity defined as the number of radioactive transformations per second that occur in a particular compound or composition that contains one or more radioactive substances. The unit of activity used herein is the becquerel (Bq), which is defined equivalent to reciprocal seconds (1/seconds or s-1).

As used herein, “activity concentration” refers to the total amount of radioactivity per unit volume. In certain embodiments, activity concentration is expressed in Bq/L or magnitudes thereof (e.g., MBq/mL).

As used herein, “radiocobalt activity content” refers to the ratio, expressed as a percentage, of the radioactivity of radiocobalt species in a composition to the total radioactivity of the composition.

As used herein, “end of synthesis” (EoS) refers to the completion of radiolabeling NODAGA-LM3 with [⁶¹Cu]CuCl₂ and subsequent preparation of the pharmaceutical composition, e.g., dilution with saline solution.

As used herein, “effective amount.” “pharmaceutically effective amount,” or “therapeutically effective amount” mean a sufficient amount of the pharmaceutical composition to provide the desired utility when administered to a subject. In certain embodiments, an effective amount includes an amount of pharmaceutical composition sufficient to generate an image of subject. In certain embodiments, an effective amount includes an amount of pharmaceutical composition sufficient to diagnose a disease in a subject. It is understood that for any given case, an appropriate “effective amount” can be determined by one of ordinary skill in the art using routine experimentation. For example, when administered in clinic, such pharmaceutical compositions will contain an amount of active ingredient effective to achieve the desired result (e.g., imaging cancerous tissue).

As used herein, “effective dose” refers to the internationally accepted central radiological risk metric quantity. The calculation of effective dose can be seen as a three-step process. First, the mean absorbed doses to organs and tissues are determined in gray (Gy; J kg⁻¹). Second, the absorbed doses are converted to equivalent doses in sievert (Sv) using tissue/organ radiation weighting factors (wR). The weighting factors were computed from radiation epidemiological data, and take into consideration that some organs (e.g., breasts, stomach, and, lungs) are more radiosensitive than others (e.g., brain and skin). The summing of organ/tissue equivalent doses, each weighted by the appropriate tissue weighting factor (wT), gives the effective dose, in Sv or Sv/MBq (i.e., the effective dose received per unit of activity administered). The effective dose is therefore a risk metric, and not a dosimetric quantity per se.

As used herein, “molar activity” refers to the amount of radioactivity (e.g., number of nuclear disintegrations per second) per unit mole of the radiolabeled compound. In certain embodiments, molar activity is expressed in Bq/mol, e.g., MBq/nmol and is used where the molecular weight of the labelled material is known. The “apparent molar activity” (apparent Am) takes into account the amounts of the radiolabeled (compound A*) and non-radiolabeled radiotracer (compound A), radiolabeled impurities (compound B*) and non-radiolabeled impurities (compound B) and remaining precursor (compound C) present expressed in mol (or μmol). See, e.g., Luurtsema, G., et al. “EANM guideline for harmonisation on molar activity or specific activity of radiopharmaceuticals: impact on safety and imaging quality.” EJNMMI Radiopharm. Chem. 6, 34 (2021).

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate. 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium, and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutical composition” refers to a composition suitable for administration to a subject that comprises [⁶¹Cu]Cu-NODAGA-LM3 and one or more pharmaceutically acceptable excipients. A “pharmaceutically acceptable excipient”, as used herein refers to for example, pharmaceutically, physiologically, acceptable organic or inorganic carrier substances suitable for intravenous administration that do not deleteriously react with [⁶¹Cu]Cu-NODAGA-LM3 or salt thereof. Exemplary pharmaceutical excipients are known to those of skill in the art.

As used herein, “positive predictive value” or PPV refers to a given method's precision in identifying cancerous lesions/tumors. A method that has a high PPV is more desirable. Mathematically, PPV is the number of true positives (TP) divided by the sum of TP and false positives (FP). In other words, PPV is TP/(TP+FP).

As used herein, “peptide receptor radionuclide therapy” (PRRT) refers to the use of one or more radiotherapeutic agents for the delivery of targeting radiation, usually by a chelated radionuclide, to a specific tissue or receptor associated with cancer. The targeting and selection of certain tissues over another is accomplished by specific peptide hormone receptors expressed or overexpressed on the surface of cancerous biomass.

As used herein, “radiochemical purity” refers to the ratio, given as a percent, of radioactivity from the radionuclide in the pharmaceutical composition (e.g., the desired radionuclide that is chelated in a radiotracer as described herein) to the total radioactivity of the composition that comprises the radiotracer. The majority of the radioactive isotope is attached to the tracer construct and is not free or attached to another chemical entity as these forms may have a different biodistribution. Radiochemical purity (RCP) measurements establish the content of impurities labelled with the same radionuclide used to prepare a radiopharmaceutical, but with a different chemical form.

Radiochemical purity is determined according to methods well known to those of skill in the art, e.g., radio-HPLC or iTLC. As is understood in the art, determination of radiochemical purity is not strictly quantitative, and it is calculated as the ratio between the peak area of the desired radiotracer and the overall area of all the detected peaks in the radiochromatogram (corrected for decay). The instrument used to determine radiochemical purity with HPLC (radio-HPLC) is a radiometric detector (radiodetector), which has an in-line detector connected in series with a UV or other physicochemical detector. The radiometric detector can be a Geiger-Müller probe, a scintillation detector, or a PIN diode. As compared with radio-HPLC it has the big advantage that all applied radioactivity is detected and there are no concerns with recovery. The radiochemical purity is reported at a particular time point, e.g., a certain time after end of synthesis (EoS, described herein). In certain embodiments, a radiotracer radiochemical purity is measured at 3 hours after end of synthesis of the radiotracer, at 6 hours after end of synthesis of the radiotracer, at 9 hours after end of synthesis of the radiotracer, or at 12 hours after end of synthesis of the radiotracer.

As used herein, “radionuclidic purity” refers to the ratio, expressed as a percentage, of the radioactivity of a particular radionuclide to the total radioactivity of the sample, e.g., the starting material used to prepare a radiotracer. In certain embodiments, radionuclidic purity can be determined by high resolution gamma spectroscopy (e.g., high-purity germanium (HPGe) detector) on a sample after expiration, e.g. >8 hours or >3 weeks) and is then extrapolated (e.g., using the TENDL-2019 database according to procedures well known in the art), and reported herein as the value at the end of synthesis (e.g., EoB+2 hours) of the radionuclide.

As used herein, “radionuclidic impurities” refer to all non-⁶¹Cu radionuclides in a composition.

As used herein, “radiotracer” refers to a compound of the present disclosure comprising a radionuclide or radioisotope. It is understood herein that when a compound, e.g., a radiotracer, is described as comprising a particular radioisotope or radionuclide (e.g., ⁶¹Cu) that the compound is isotopically enriched in that isotope at the indicated position.

As used herein, “sensitivity” refers to the ability of a given method to accurately identify cancerous lesions/tumors. A method that has high sensitivity is more desirable. Mathematically, sensitivity is the number of true positives (TP) divided by the sum of the number of TPs and false positives (FP). In other word, sensitivity (Se)=TP/(TP+FN).

As used herein, “specific activity” refers to the activity of a radionuclide per unit mass or per unit volume. In certain embodiments, specific activity refers to the activity of a radionuclide per unit mass of the radiotracer. In certain embodiments, specific activity refers to the activity of a radionuclide per unit mass of the [⁶¹Cu]CuCl₂ solution used to radiolabel the NODAGA-LM3. In certain embodiments, specific activity refers to the activity of a radionuclide per unit volume of the [⁶¹Cu]CuCl₂ solution used to radiolabel the NODAGA-LM3. In certain embodiments, specific activity is measured at EoS.

As used herein, “subject” refers to the person or organism to which the composition is, or is intended to be, administered. As such, subjects of the present disclosure may include but are not limited to mammals, e.g., humans and other primates, such as chimpanzees and other ape and monkey species. In preferred embodiments the subject are humans. The term subject includes a person or organism of any age, weight, or other physical characteristic, including an adult, an adolescent, a child, an infant or a newborn.

### 4.2. Pharmaceutical Compositions

In one aspect, the present disclosure provides pharmaceutical compositions comprising a radiotracer ([*Cu]Cu-NODAGA-LM3) that has the structure

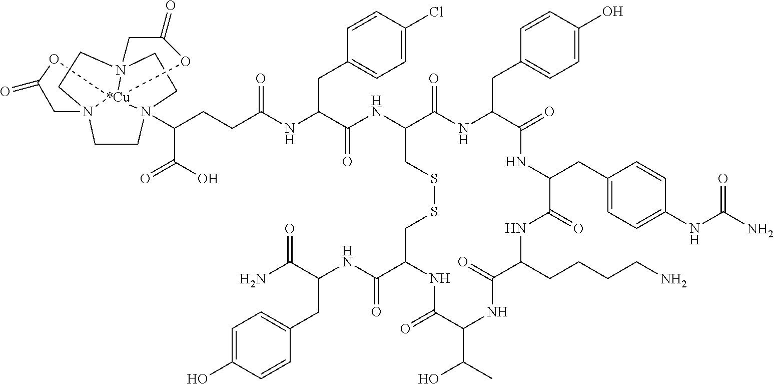

or is a pharmaceutically acceptable salt thereof, wherein *Cu is selected from ⁶¹Cu, ⁶⁴Cu, and ⁶⁷Cu, particularly ⁶¹Cu.

The pharmaceutical compositions are useful in the methods described herein, e.g., for generating radiographic images of subjects diagnosed with or suspected of having a somatostatin receptor subtype 2 (SST2)-expressing tumor following administration of the pharmaceutical composition, e.g., by intravenous (i.v.) administration.

⁶¹Cu-NODAGA-LM3 is a promising imaging agent for SST2-expressing tumors that combines three novel properties: (1) the use of an isotope (⁶¹Cu) with favorable physical characteristics (longer half-life and lower positron energy than ⁶⁸Ga) and almost ideal properties for central production and distribution (t1/2=3.33 h), (2) higher tumor uptake and longer retention of the SST2 antagonist LM3 over established agonists, and (3) a metabolically stable chelation with NODAGA which takes full advantage of delayed ⁶¹Cu-NODAGA-LM3 PET imaging with higher image contrast.

In certain embodiments, the radiotracer has the structure

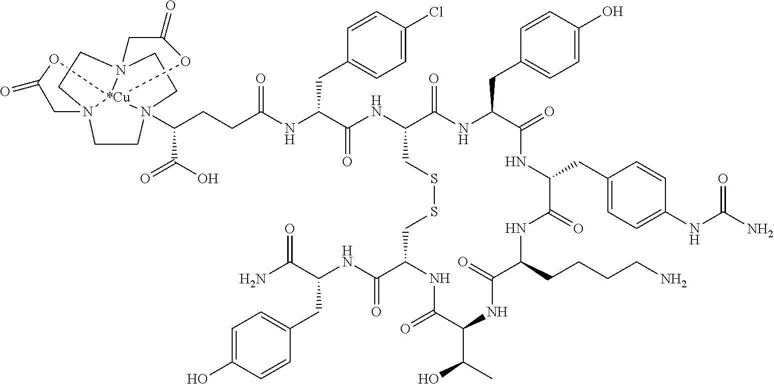

or is a pharmaceutically acceptable salt thereof, wherein *Cu is selected from ⁶¹Cu, ⁶⁴Cu, and ⁶⁷Cu, particularly ⁶¹Cu.

In certain embodiments, the pharmaceutical composition comprises the radiotracer in an amount ≥1 μg, e.g., ≥10 μg, ≥20 μg, ≥30 μg, ≥40 μg, ≥50 μg, ≥60 μg, ≥70 μg, or ≥80 μg.

In certain embodiments, the pharmaceutical composition comprises the radiotracer in an amount from 1 μg to 100 μg, e.g., from 20 μg to 90 μg, from 20 μg to 80 μg, from 20 μg to 70 μg, from 20 μg to 60 μg, from 20 μg to 50 μg, from 20 μg to 40 μg, from 20 μg to 30 μg, from 30 μg to 100 μg, from 30 μg to 90 μg, from 30 μg to 80 μg, from 30 μg to 70 μg, from 30 μg to 60 μg, from 30 μg to 50 μg, from 30 μg to 40 μg, from 40 μg to 100 μg, from 40 μg to 90 μg, from 40 μg to 80 μg, from 40 μg to 70 μg, from 40 μg to 60 μg, from 40 μg to 50 μg, from 50 μg to 100 μg, from 50 μg to 90 μg, from 50 μg to 80 μg, from 50 μg to 70 μg, from 50 μg to 60 μg, from 60 μg to 100 μg, from 60 μg to 90 μg, from 60 μg to 80 μg, from 60 μg to 70 μg, from 70 μg to 100 μg, from 70 μg to 90 μg, from 70 μg to 80 μg, from 80 μg to 100 μg, from 80 μg to 90 μg, or from 90 μg to 100. In certain embodiments, the pharmaceutical composition comprises from 20 μg to 100 μg, from 20 μg to 50 μg, or from 20 μg to 40 μg of the radiotracer. In certain embodiments, the pharmaceutical composition comprises 1 μg to 50 μg of the radiotracer, e.g., from 1 μg to 30 μg, from 1 μg to 10 μg, or from 1 μg to 5 μg.

In certain embodiments, the pharmaceutical composition is in the form of a liquid, e.g., a solution, such as an aqueous solution.

In certain embodiments, the pharmaceutical composition comprises an aqueous vehicle, i.e., a medium or carrier comprising at least a minimal amount of water, in which all the other components are dissolved. Exemplary aqueous vehicles include, e.g., deionized water, saline, phosphate buffer, citrate buffer, malate buffer, tartrate buffer, balanced salt solution, salts of organic acids, combinations of organic acids and salts of organic acids (e.g., tribasic sodium citrate and citric acid, malic acid and sodium malate, and potassium sodium tartrate and tartaric acid), and combinations thereof. In certain embodiments, the aqueous vehicle comprises saline solution, e.g., isotonic saline solution.

In certain embodiments, the pharmaceutical composition has a total volume of 2 to 15 mL, e.g., from 2 mL to 14 mL, from 2 mL to 12 mL, from 2 mL to 10 mL, from 2 mL to 8 mL, from 2 mL to 6 mL, from 2 mL to 4 mL, from 4 mL to 15 mL, from 4 mL to 14 mL, from 4 mL to 12 mL, from 4 mL to 10 mL, from 4 mL to 8 mL, from 4 mL to 6 mL, from 6 mL to 15 mL, from 6 mL to 14 mL, from 6 mL to 12 mL, from 6 mL to 10 mL, from 8 mL to 15 mL, from 8 mL to 14 mL, from 8 mL to 12 mL, from 8 mL to 10 mL, from 12 mL to 15 mL, or from 12 mL to 14 mL. In certain embodiments, the pharmaceutical composition has a total volume from 2 mL to 5 mL or from 8 to 12 mL.

The total volume of the pharmaceutical composition comprises one or more unit doses for administration to the subject in the methods described herein, e.g., one dose, two doses, or three or more doses. In certain embodiments, the pharmaceutical composition comprises from 2 mL to 5 mL, corresponding to one unit dose for administration. In certain embodiments, the pharmaceutical composition comprises from 8 mL to 12 mL, corresponding to two unit doses for administration.

Exemplary radiolytic inhibitors include, but are not limited to, ascorbic acid, gentisic acid, citric acid, N-tert-butyl-α-phenylnitrone (PBN), polyvinylpyrrolidone (PVP), ethanol, DMSA, cysteine, vanillin, methionine, adenine, dobesilic acid, thymine, uracil, nicotinic acid, nicotinamide, salts of any of the foregoing, and combinations thereof.

In certain embodiments, the radiolytic inhibitor comprises ascorbic acid or a salt thereof. In certain embodiments, the radiolytic inhibitor comprises ascorbic acid. In certain embodiments, the radiolytic inhibitor comprises a salt of ascorbic acid, e.g., sodium ascorbate, calcium ascorbate, or potassium ascorbate.

In certain embodiments, the radiolytic inhibitor is present in the pharmaceutical composition in a concentration ≥1 mg/mL, e.g., ≥2 mg/mL, ≥3 mg/mL, or ≥5 mg/mL.

In certain embodiments, the radiolytic inhibitor is present in the pharmaceutical composition in a concentration from 1 mg/mL to 10 mg/mL, e.g., from 1 mg/mL to 7 mg/mL, from 1 mg/mL to 5 mg/mL, from 1 mg/mL to 3 mg/mL, or from 1 mg/mL to 2 mg/mL.

### 4.2.1. Properties

In certain embodiments, the pharmaceutical composition has an activity of ≥25 MBq. e.g., ≥30 MBq, ≥50 MBq, ≥75 MBq, ≥100 MBq, ≥150 MBq, ≥200 MBq, ≥250 MBq, ≥300 MBq, ≥350 MBq, ≥400 MBq, ≥450 MBq, ≥500 MBq, ≥750 MBq, or ≥1,000 MBq.

In certain embodiments, the pharmaceutical composition has an activity from 25 MBq to 1,500 MBq, e.g., from 25 MBq to 1,000 MBq, from 25 MBq to 750 MBq, from 25 MBq to 500 MBq, from 25 MBq to 300 MBq, from 25 MBq to 200 MBq, from 25 MBq to 100 MBq, from 50 MBq to 1,500 MBq, from 50 MBq to 1,000 MBq, from 50 MBq to 750 MBq, from 50 MBq to 500 MBq, from 50 MBq to 300 MBq, from 50 MBq to 200 MBq, or from 50 MBq to 100 MBq. In certain embodiments, the pharmaceutical composition has an activity from 100 MBq to 300 MBq. In certain embodiments, the pharmaceutical composition has an activity from 100 MBq to 200 MBq.

In certain embodiments, the pharmaceutical composition has an activity from 700 MBq to 1,500 MBq. e.g., from 700 MBq to 1,200 MBq, from 700 MBq to 1,000 MBq, from 750 MBq to 1,500 MBq, from 750 MBq to 1,200 MBq, from 750 MBq to 1,000 MBq, from 1,000 MBq to 1,500 MBq, from 1,200 MBq to 1,500 MBq, or from 1,200 MBq to 1,500 MBq.

In certain embodiments, the pharmaceutical composition has an activity of ≥50 MBq, e.g., ≥80 MBq, ≥100 MBq, ≥200 MBq, ≥300 MBq, ≥400 MBq, ≥500 MBq, or ≥600 MBq.

In certain embodiments, the pharmaceutical composition has an activity from 50 MBq to 1,000 MBq. e.g., from 50 MBq to 900 MBq, from 50 MBq to 800 MBq, from 50 MBq to 700 MBq, from 50 MBq to 600 MBq, from 50 MBq to 500 MBq, from 50 MBq to 400 MBq, from 50 MBq to 300 MBq, from 50 MBq to 200 MBq, from 50 MBq to 100 MBq, from 100 MBq to 1,000 MBq, from 100 MBq to 900 MBq, from 100 MBq to 800 MBq, from 100 MBq to 700 MBq, from 100 MBq to 600 MBq, from 100 MBq to 500 MBq, from 100 MBq to 400 MBq, from 100 MBq to 300 MBq, from 100 MBq to 200 MBq, from 200 MBq to 1,000 MBq, from 200 MBq to 900 MBq, from 200 MBq to 800 MBq, from 200 MBq to 700 MBq, from 200 MBq to 600 MBq, from 200 MBq to 500 MBq, from 200 MBq to 400 MBq, from 200 MBq to 300 MBq, from 300 MBq to 1,000 MBq, from 300 MBq to 900 MBq, from 300 MBq to 800 MBq, from 300 MBq to 700 MBq, from 300 MBq to 600 MBq, from 300 MBq to 500 MBq, from 300 MBq to 400 MBq, from 400 MBq to 1,000 MBq, from 400 MBq to 900 MBq, from 400 MBq to 800 MBq, from 400 MBq to 700 MBq, from 400 MBq to 600 MBq, from 400 MBq to 500 MBq, from 500 MBq to 1,000 MBq, from 500 MBq to 900 MBq, from 500 MBq to 800 MBq, from 500 MBq to 700 MBq, from 500 MBq to 600 MBq, from 600 MBq to 1,000 MBq, from 600 MBq to 900 MBq, from 600 MBq to 800 MBq, from 600 MBq to 700 MBq, from 700 MBq to 1,000 MBq, from 700 MBq to 900 MBq, from 700 MBq to 800 MBq, from 800 MBq to 1,000 MBq, from 800 MBq to 900 MBq, or from 900 MBq to 1,000 MBq.

In certain embodiments, the pharmaceutical composition has an activity from 200 MBq to 800 MBq. In certain embodiments, the pharmaceutical composition has an activity from 500 MBq to 700 MBq.

In certain embodiments, the pharmaceutical composition has a radiotracer radiochemical purity of ≥90% at 12 hours after end of synthesis, e.g., ≥93%, ≥95%, ≥96%, ≥97%, ≥98%, or ≥99%.

In certain embodiments, the pharmaceutical composition has a radiotracer radiochemical purity from 90% to 99% at 12 hours after end of synthesis, e.g., from 90% to 98%, from 90% to 97%, from 90% to 95%, from 90% to 93%, from 93% to 99%, from 93% to 98%, from 93% to 97%, from 93% to 95%, from 95% to 99%, from 95% to 98%, from 95% to 97%, from 97% to 99%, or from 98% to 99%.

In certain embodiments, the pharmaceutical composition has a ⁶¹[Cu]Cu radionuclidic purity at end of synthesis of ≥95%. e.g., ≥96%, ≥97%, ≥98%, or ≥99%. In certain embodiments, the pharmaceutical composition has a ⁶¹[Cu]Cu radionuclidic purity at end of synthesis of ≥97%.

In certain embodiments, the pharmaceutical composition has a ⁶¹[Cu]Cu radionuclidic purity at end of synthesis of ≥98%, e.g., ≥99%, ≥99.5%, ≥99.6%, ≥99.7%, ≥99.8%, or ≥9.9%. In certain embodiments, the pharmaceutical composition has a ⁶¹[Cu]Cu radionuclidic purity at end of synthesis of ≥99.99%. In certain embodiments, the pharmaceutical has a radiotracer radiochemical purity ≥99% at 3 hours after end of synthesis, e.g., 4 hours after end of synthesis, 5 hours after end of synthesis, 6 hours after end of synthesis, 9 hours after end of synthesis, 12 hours after end of synthesis, or 18 hours after end of synthesis.

In certain embodiments, the pharmaceutical composition has a ⁶¹[Cu]Cu radionuclidic purity at end of synthesis of ≥95%, e.g., ≥96%, ≥97%, ≥98%, or ≥99%. In certain embodiments, the pharmaceutical composition has a ⁶¹[Cu]Cu radionuclidic purity at end of synthesis of ≥97%.

In certain embodiments, the pharmaceutical composition has a ⁶¹[Cu]Cu radionuclidic purity at end of synthesis of ≥98%, e.g., ≥99%, ≥99.5%, ≥99.6%, ≥99.7%, ≥99.8%, or ≥99.9%. In certain embodiments, the pharmaceutical composition has a ⁶¹[Cu]Cu radionuclidic purity at end of synthesis of ≥99.99%.

In certain embodiments, the pharmaceutical composition has a radiocobalt activity content at end of synthesis of ≤0.1%, e.g., ≤0.05%, ≤0.02%, ≤0.01%, ≤0.005% or ≤0.001%.

In certain embodiments, the pharmaceutical composition has at least one of a 56 [Co]Co specific activity or ⁵⁸[Co]Co specific activity at end of synthesis of ≤1,500 Bq/g, e.g., ≤1,200 Bq/g, ≤1,000 Bq/g, ≤800 Bq/g, ≤400 Bq/g, or ≤200 Bq/g.

In certain embodiments, the pharmaceutical composition has at least one of a 56 [Co]Co specific activity or ⁵⁸[Co]Co specific activity at end of synthesis from 200 Bq/g to 1,500 Bq/g. e.g., from 200 Bq/g to 1,200 Bq/g, from 200 Bq/g to 1,000 Bq/g, from 200 Bq/g to 800 Bq/g, from 200 to 600 Bq/g, or from 200 Bq/g to 400 Bq/g.

In certain embodiments, the pharmaceutical composition has a 56 [Co]Co specific activity of ≤100 Bq/g. e.g., ≤50 Bq/g, ≤25 Bq/g, ≤10 Bq/g, ≤8 Bq/g, <4 Bq/g, or ≤2 Bq/g.

In certain embodiments, the pharmaceutical composition has a 56 [Co]Co specific activity of from 1 Bq/g to 100 Bq/g, e.g., from 1 Bq/g to 50 Bq/g, from 1 Bq/g to 25 Bq/g, from 1 Bq/g to 10 Bq/g, from 1 Bq/g to 8 Bq/g, or from 1 Bq/g to 4 Bq/g.

In certain embodiments, the pharmaceutical composition has a ⁵⁸[Co]Co specific activity of ≤100 Bq/g, e.g., ≤50 Bq/g, ≤25 Bq/g, ≤10 Bq/g, ≤8 Bq/g, ≤4 Bq/g, or ≤2 Bq/g.

In certain embodiments, the pharmaceutical composition has a ⁵⁸[Co]Co specific activity of from 1 Bq/g to 100 Bq/g, e.g., from 1 Bq/g to 50 Bq/g, from 1 Bq/g to 25 Bq/g, from 1 Bq/g to 10 Bq/g, from 1 Bq/g to 8 Bq/g, or from 1 Bq/g to 4 Bq/g.

In certain embodiments, the pharmaceutical composition is characterized by a ^{110m}Ag specific activity≤0.1 Bq/g. In certain embodiments, the pharmaceutical composition is characterized by a ^{108m}Ag specific activity≤0.1 Bq/g. In certain embodiments, the pharmaceutical composition is characterized by a ¹⁰⁹Cd specific activity≤0.1 Bq/g.

In certain embodiments, the pharmaceutical composition is characterized by at least two of: a ^{110m}Ag specific activity≤0.1 Bq/g, a ^{108m}Ag specific activity≤0.1 Bq/g, and a ¹⁰⁹Cd specific activity≤0.1 Bq/g. In certain embodiments, the pharmaceutical composition is characterized by a ^{110m}Ag specific activity of ≤0.1 Bq/g, a ^{108m}Ag specific activity≤0.1 Bq/g, and a ¹⁰⁹Cd specific activity≤0.1 Bq/g.

In certain embodiments, the sum of the specific activities of the radionuclidic impurities in the pharmaceutical composition is ≤8,000 Bq/g, e.g., ≤5,000 Bq/g, ≤3,000 Bq/g, ≤1200 Bq/g, ≤ 1,000 Bq/g, ≤800 Bq/g, or ≤500 Bq/g.

In certain embodiments, the pharmaceutical composition comprises Al in an amount ≤1.2 ng/MBq. In certain embodiments, the pharmaceutical composition comprises Co in an amount ≤ 0.2 ng/MBq. In certain embodiments, the pharmaceutical composition comprises Fe in an amount ≤1.7 ng/MBq. In certain embodiments, the pharmaceutical composition comprises Pb in an amount ≤0.8 ng/MBq. In certain embodiments, the pharmaceutical composition comprises Zn in an amount ≤0.8 ng/MBq. In certain embodiments, the pharmaceutical composition comprises one or more of: an amount of Al≤1.2 ng/MBq, an amount of Co≤0.2 ng/MBq, an amount of Fe≤1.7 ng/MBq, an amount of Pb≤0.8 ng/MBq, or an amount of Zn≤0.8 ng/MBq.

In certain embodiments, the pharmaceutical composition has an activity concentration ≥10 MBq/mL. e.g., ≥20 MBq/mL, ≥30 MBq/mL, ≥40 MBq/mL, ≥50 MBq/mL, ≥60 MBq/mL, ≥70 MBq/mL, ≥80 MBq/mL, or ≥90 MBq/mL.

In certain embodiments, the pharmaceutical composition has an activity concentration from 10 MBq/mL to 100 MBq/mL, e.g., from 10 MBq/mL to 50 MBq/mL or from 40 MBq/mL to 90 MBq/mL.

In certain embodiments, the pharmaceutical composition has an activity concentration from 10 MBq/mL to 60 MBq/mL, e.g., from 10 MBq/mL to 50 MBq/mL, from 10 MBq/mL to 40 MBq/mL, from 10 MBq/mL to 30 MBq/mL, from 10 MBq/mL to 20 MBq/mL, from 20 MBq/mL to 60 MBq/mL, from 20 MBq/mL to 50 MBq/mL, from 20 MBq/mL to 40 MBq/mL, from 20 MBq/mL to 30 MBq/mL, from 30 MBq/mL to 60 MBq/mL, from 30 MBq/mL to 50 MBq/mL, from 30 MBq/mL to 40 MBq/mL, from 40 MBq/mL to 60 MBq/mL, from 40 MBq/mL to 50 MBq/mL, or from 50 MBq/mL to 60 MBq/mL.

In certain embodiments, the apparent molar activity of the radiotracer is ≥1 MBq/nmol, e.g., ≥10 MBq/nmol, ≥20 MBq/nmol, ≥30 MBq/nmol, or ≥50 MBq/nmol.

In certain embodiments, the apparent molar activity of the radiotracer is from 1 MBq/nmol to 50 MBq/nmol, e.g., from 1 MBq/nmol to 30 MBq/nmol, from 1 MBq/nmol to 20 MBq/nmol, from 1 MBq/nmol to 10 MBq/nmol, from 10 MBq/nmol to 50 MBq/nmol, from 10 MBq/nmol to 30 MBq/nmol, from 10 MBq/nmol to 20 MBq/nmol, from 20 MBq/nmol to 50 MBq/nmol, from 20 MBq/nmol to 40 MBq/nmol, or from 20 MBq/nmol to 30 MBq/nmol.

In certain embodiments, the molar activity of the radiotracer is ≥1 MBq/nmol, e.g., ≥10 MBq/nmol, ≥20 MBq/nmol, ≥30 MBq/nmol, or ≥50 MBq/nmol.

In certain embodiments, the molar activity of the radiotracer is from 1 MBq/nmol to 50 MBq/nmol, e.g., from 1 MBq/nmol to 30 MBq/nmol, from 1 MBq/nmol to 20 MBq/nmol, from 1 MBq/nmol to 10 MBq/nmol, from 10 MBq/nmol to 50 MBq/nmol, from 10 MBq/nmol, to 30 MBq/nmol, from 10 MBq/nmol to 20 MBq/nmol, from 20 MBq/nmol to 50 MBq/nmol, from 20 MBq/nmol to 40 MBq/nmol, or from 20 MBq/nmol to 30 MBq/nmol.

In certain embodiments, the molar activity of the radiotracer is from 1 MBq/nmol to 600 MBq/nmol, e.g., from 1 MBq/nmol to 600 MBq/nmol, from 1 MBq/nmol to 500 MBq/nmol, from 1 MBq/nmol to 400 MBq/nmol, from 1 MBq/nmol to 300 MBq/nmol, from 1 MBq/nmol to 200 MBq/nmol, from 1 MBq/nmol to 100 MBq/nmol, from 30 MBq/nmol to 600 MBq/nmol, from 30 MBq/nmol to 500 MBq/nmol, from 30 MBq/nmol to 400 MBq/nmol, from 30 MBq/nmol to 300 MBq/nmol, from 30 MBq/nmol to 200 MBq/nmol, from 30 MBq/nmol to 100 MBq/nmol, from 30 MBq/nmol, from 100 MBq/nmol to 600 MBq/nmol, from 100 MBq/nmol to 500 MBq/nmol, from 100 MBq/nmol to 400 MBq/nmol, from 100 MBq/nmol to 300 MBq/nmol, or from 100 MBq/nmol to 200 MBq/nmol.

In certain embodiments, the molar activity of the radiotracer is from 1 MBq/nmol to 40 MBq/nmol, e.g., from 1 MBq/nmol to 30 MBq/nmol, from 1 MBq/nmol to 20 MBq/nmol, from 1 MBq/nmol to 10 MBq/nmol, from 5 MBq/nmol to 40 MBq/nmol from 5 MBq/nmol to 30 MBq/nmol, from 5 MBq/nmol to 20 MBq/nmol, from 5 MBq/nmol to 10 MBq/nmol, from 10 MBq/nmol to 40 MBq/nmol, from 10 MBq/nmol to 30 MBq/nmol, or from 10 MBq/nmol to 20 MBq/nmol.

In certain embodiments, the pH of the pharmaceutical composition is from 5 to 7, e.g., from 5 to 6, 5.5 to 6.5, or from 6 to 7. In certain embodiments, the pH of the pharmaceutical composition is 5, 6, or 7+/−0.1.

### 4.2.2. Excipients

In certain embodiments, the pharmaceutical composition comprises one or more excipients.

In certain embodiments, the pharmaceutical composition comprises sodium chloride. In certain embodiments, sodium chloride is present in a concentration from 0.5 mg/mL to 50 mg/mL, e.g., from 5 mg/mL to 10 mg/mL, from 10 mg/mL to 30 mg/mL, or from 20 mg/mL to 50 mg/mL.

In certain embodiments, the pharmaceutical composition comprises ethanol. In certain embodiments, ethanol is present in an amount ≤10% v/v, e.g., ≤8% v/v, ≤5% v/v, or ≤3% v/v.

In certain embodiments, the pharmaceutical composition comprises ascorbic acid or a salt thereof, ethanol, and sodium chloride.

In certain embodiments, the pharmaceutical composition comprises 1-100 μg radiotracer, 5-20 mg ascorbic acid, 0.1-1 mL ethanol, and 1-10 mL isotonic saline solution.

In certain embodiments, the pharmaceutical composition comprises 20-30 μg radiotracer, 5-10 mg ascorbic acid, 0.1-0.5 mL ethanol, and 1-5 mL isotonic saline solution.

In certain embodiments, the pharmaceutical composition comprises 50-75 μg radiotracer, 15-20 mg ascorbic acid, 0.5-1 mL ethanol, and 5-10 mL isotonic saline solution.

In certain embodiments, the pharmaceutical composition comprises ethanol, ascorbic acid, sodium chloride, and one or more of a metal scavenger (e.g., sodium EDTA), sodium bicarbonate, and propylene glycol. In certain embodiments, the pharmaceutical composition comprises ethanol, ascorbic acid, sodium chloride, and two or more of a metal scavenger (e.g., sodium EDTA), sodium bicarbonate, and propylene glycol. In certain embodiments, the pharmaceutical composition comprises ethanol, ascorbic acid, sodium chloride, a metal scavenger (e.g., sodium EDTA), sodium bicarbonate, and propylene glycol.

In certain embodiments, the pharmaceutical composition comprises sodium bicarbonate. In certain embodiments, the pharmaceutical composition comprises from 0.5 to 10 mg of sodium bicarbonate, e.g., from 0.5 mg to 7.5 mg, from 0.5 mg to 5 mg, from 0.5 to 2.5 mg, from 2.5 mg to 10 mg, from 2.5 mg to 7.5 mg, from 2.5 mg to 5 mg, from 5 mg to 10 mg, from 5 mg to 7.5 mg, or from 5 mg to 10 mg. In certain embodiments, the pharmaceutical composition comprises sodium bicarbonate in an amount from 1 mg to 3 mg or 0.5 mg to 2 mg.

In certain embodiments, the pharmaceutical composition comprises a metal scavenger, e.g., EDTA or a salt thereof. In certain embodiments, the metal scavenger is sodium EDTA. In certain embodiments, the pharmaceutical composition comprises a metal scavenger (e.g., sodium EDTA) in an amount from 0.001 mg to 0.01 mg, e.g., from 0.001 mg to 0.0075 mg, from 0.001 mg to 0.005 mg, from 0.001 mg to 0.0025 mg, from 0.0025 mg to 0.01 mg, from 0.0025 mg to 0.0075, from 0.0025 mg to 0.005 mg, from 0.005 mg to 0.01 mg, from 0.005 mg to 0.0075 mg, or from 0.0075 mg to 0.01 mg. In certain embodiments, the pharmaceutical composition comprises a metal scavenger (e.g., sodium EDTA) in an amount from 0.0025 mg to 0.0075 mg or from 0.001 mg to 0.005 mg.

In certain embodiments, the pharmaceutical composition comprises propylene glycol. In certain embodiments, the pharmaceutical composition comprises propylene glycol in an amount from 5 mg to 100 mg, e.g., from 5 mg to 75 mg, from 5 mg to 50 mg, from 5 mg to 25 mg, from 25 mg to 75 mg, from 25 mg to 50 mg, from 50 mg to 75 mg, or from 50 mg to 100 mg. In certain embodiments, the pharmaceutical composition comprises propylene glycol in an amount from 10 mg to 50 mg, e.g., from 30 mg to 50 mg or from 10 mg to 20 mg.

In certain embodiments, the pharmaceutical composition comprises ethanol, ascorbic acid, sodium chloride, and one or more of a metal scavenger (e.g., sodium EDTA), sodium bicarbonate, and propylene glycol. In certain embodiments, the pharmaceutical composition comprises ethanol, ascorbic acid, sodium chloride, and two or more of a metal scavenger (e.g., sodium EDTA), sodium bicarbonate, and propylene glycol. In certain embodiments, the pharmaceutical composition comprises ethanol, ascorbic acid, sodium chloride, a metal scavenger (e.g., sodium EDTA), sodium bicarbonate, and propylene glycol.

### 4.3. Methods of Imaging

In one aspect, the present disclosure provides methods for imaging subjects suspected of having or diagnosed with SST2-expressing tumors comprising administering an effective amount of a pharmaceutical composition of the present disclosure and generating one or more radiographic images of the subject.

In another aspect, the present disclosure provides methods for determining a subject's response to a cancer treatment over time, e.g., a subject previously diagnosed with a SST2-expressing tumor. In certain embodiments, the method comprises:
(a) administering an effective amount of a pharmaceutical composition of the present disclosure to a subject diagnosed with a SST2-expressing tumor at an earlier time point and at a later time point;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

In certain embodiments, the subject is a mammal. In certain embodiments, the subject is a human. In certain embodiments, the subject is a human ≥18 years of age.

In certain embodiments, the subject has been diagnosed with one or more SST2-expressing tumors. In certain embodiments, the subject has been diagnosed with a bronchopulmonary neuroendocrine tumor (BP NET). In certain embodiments, the subject has been diagnosed with a gastroenteropancreatic neuroendocrine tumor (GEP NET).

In certain embodiments, the subject is suspected of having, but not diagnosed with, one or more SST2-expressing tumors. In certain embodiments, the subject is suspected of having, but not diagnosed with, a BP NET. In certain embodiments, the subject is suspected of having, but not diagnosed with, a GEP NET.

In certain embodiments, one or more SST2 lesions are detected in the subject prior to administering the pharmaceutical composition described herein, e.g., two or more lesions or three or more lesions. In certain embodiments, three or more SST2 lesions are detected in the subject prior to administering the pharmaceutical composition described herein.

In certain embodiments, the subject has an estimated glomerular filtration rate (eGFR) (CKD-EPI) of ≥45 mL/min.

In certain embodiments, the pharmaceutical composition is not administered less than one week after a first radiotracer that is not [⁶¹Cu]Cu-NODAGA-LM3 or a pharmaceutically acceptable salt thereof. Exemplary first radiotracers include [⁶⁸Ga]Ga-NODAGA-LM3, [68Ga]Ga-DOTA-LM3, and [⁶⁸Ga]Cu-NODAGA-JR11.

In certain embodiments, the pharmaceutical composition is not administered less than one week after a SSTR-targeting ligand, e.g., somatostatin agonists or somatostatin antagonists. Exemplary somatostatin agonists include, e.g., pentetreotide, TOC derivatives (e.g., DOTATOC), TATE derivatives (e.g., DOTATATE). Exemplary somatostatin antagonists include, e.g., derivatives of JR11 and BASS.

In certain embodiments, the subject is receiving cancer treatment at the time of administering the pharmaceutical composition or has received cancer treatment in the last five years prior to administering the pharmaceutical composition, e.g., at least 1 week, at least 1 month, at least 3 months, at least 6 months, at least 1 year, at least 2 years, at least 3 years, at least 4 years, or 5 years.

In certain embodiments, the cancer treatment comprises surgery, chemotherapy, drug therapy, peptide receptor radionuclide therapy (PRRT), gene therapy, cryotherapy, or a combination thereof.

In certain embodiments, the cancer treatment comprises drug therapy.

In certain embodiments, drug therapy comprises administration of an mTOR (mammalian target of rapamycin) inhibitor, rapamyacin, or rapalog (e.g., everolimus, temsirolimus, and ridaforolimus).

In certain embodiments, drug therapy comprises administration of a somatostatin analog. Exemplary somatostatin analogs include, but are not limited to, octapeptide octreotide, octreotide, lanreotide, and pasireotide.

In certain embodiments, drug therapy comprises administration of a combination of an mTOR inhibitor and a somatostatin analog.

In certain embodiments, the cancer treatment comprises surgery. In certain embodiments, the subject has undergone more than one surgery.

In certain embodiments, the surgery is a resection surgery, i.e., complete removal of the NET tumor. Exemplary resection surgeries, include, but are not limited to, endoscopic excision, bowel resection, gastric resection or gastrectomy, appendectomy, whipple procedure, distal pancreatectomy, lung resection, total thydroidectomy, and liver resection.

In certain embodiments, the surgery is a lymph node dissection, which is often done at the same time as a resection.

In certain embodiments, the surgery is cytoreductive surgery, i.e., removal of a large amount of cancer/as much of the cancer as possible.

In certain embodiments, the cancer treatment comprises chemotherapy. Chemotherapeutic agents useful for treating NETs is known in the art (Das S, Al-Toubah T. Strosberg J. Chemotherapy in Neuroendocrine Tumors. Cancers (Basel). 2021 Sep. 29; 13 (19): 4872.). Chemotherapeutic regimens for treatment of SSTR2-expressing tumors or NETs often involves combination therapy, as detailed below. The various combinations of compounds can be coadministered e.g., simultaneous or sequential administration of the compounds individually or in combination (more than one compound).

In certain embodiments, chemotherapy comprises administration of an alkylating agent. In certain embodiments, chemotherapy comprises administration of platinum agents.

In certain embodiments, chemotherapy comprises administration of streptozocin, optionally in combination with fluorouracil, doxorubicin, chlorozotocin, everolimus, bevacizumab, capecitabine, temozolomide, or a combination thereof.

In certain embodiments, chemotherapy comprises administration of dacarbazine, optionally in combination with fluorouracil, leucovorin, or a combination thereof.

In certain embodiments, chemotherapy comprises administration of temozolomide, optionally in combination with fluorouracil, leucovorin, thalidomide, bevacizumab, capecitabine, or a combination thereof. In certain embodiments, chemotherapy comprises coadministration of capecitabine and temozolomide.

In certain embodiments, chemotherapy comprises administration of oxaliplatin, optionally in combination with fluorouracil, capecitabine, bevacizumab, leucovorin, or a combination thereof.

In certain embodiments, chemotherapy comprises cisplatin or carboplatin plus etoposide.

In certain embodiments, the cancer treatment comprises PRRT, i.e., systemic radiotherapy that allows targeted administration of radiopharmaceuticals nuclides to tumor cells expressing high levels of SSTR2. Exemplary PRRT radiopharmaceuticals include, but are not limited to, [¹⁷⁷Lu]Lu-DOTATE, [¹⁷⁷Lu]Lu-DOTATATE (LUTATHERA®), [⁹⁰Y]Y-DOTATOC, [²¹²Pb]Pb-DOTAM-TATE, [²¹³Bi]Bi-DOTA-TOC, [²²⁵Ac]Ac-DOTA-TATE, [¹¹¹In]In-DOTA-SST-ANT, [¹⁷⁷Lu]Lu-DOTA-BASS, [¹⁷⁷Lu]Lu-DOTA-JR11 (OPS201), and [¹⁷⁷Lu]Lu-DOTA-LM3, [177Yb]Yb-DOTA-LM3, [225 Ac]Ac-DOTA-LM3, [212Pb]Pb-DOTA-LM3, [177Yb]Yb-DOTA-TATE, [²¹²Pb]Pb-DOTA-TATE, [⁹⁰Y]Y-DOTA-JR11, [225 Ac]Ac-DOTA-JR11, [177Lu]Lu-DOTA-TOC. [⁹⁰Y]Y-DOTA-TOC. [²²⁵Ac]Ac-DOTA-TOC. [²¹³Bi]Bi-DOTA-TOC, [¹⁷⁷Lu]Lu-DOTA-EB-TATE, [⁹⁰Y]Y-DOTALAN, and [²¹²Pb]Pb-VMT-α-NET.

In certain embodiments, the cancer treatment comprises chemotherapy and PRRT. In certain embodiments, combined chemotherapy and PRRT comprises administration of any of the above-mentioned chemotherapy agents in combination with any of the above-mentioned PRRT agents. In certain embodiments, combined chemotherapy and PRRT comprises administration of (i) capecitabine, temozolomide, or a combination thereof and (ii) [¹⁷⁷Lu]Lu-DOTATATE.

In certain embodiments, the pharmaceutical composition is administered intravenously. In certain embodiments, administration occurs as a single bolus infusion. In certain embodiments, administration occurs over two or more separate infusions.

In certain embodiments, the pharmaceutical composition is administered over a period of time of at least 10 seconds, e.g., at least 15 seconds, at least 20 seconds, at least 30 seconds, at least 1 minute, at least 5 minutes, or at least 10 minutes.

In certain embodiments, the pharmaceutical composition is administered over a period of time from 10 seconds to 10 minutes, e.g., from 10 seconds to 5 minutes, from 10 seconds to 1 minute, from 10 seconds to 30 second, from 10 seconds to 20 seconds, from 20 seconds to 10 minutes, from 20 seconds to 5 minutes, from 20 seconds to 1 minute, from 20 seconds to 30 seconds, from 30 seconds to 10 minutes, from 30 seconds to 5 minutes, from 30 seconds to 1 minute, from 1 minute to 10 minutes, from 1 minute to 5 minutes, or from 5 minutes to 10 minutes.

In certain embodiments, the effective dose per MBq of administered pharmaceutical composition is ≤20 μSv/MBq. e.g., ≤15 μSv/MBq, ≤10 μSv/MBq, ≤7.5 μSv/MBq, ≤5 μSv/MBq, or ≤3 μSv/MBq.

In certain embodiments, the effective dose per MBq of administered pharmaceutical composition is from 3 μSv/MBq to 20 μSv/MBq, e.g., from 3 μSv/MBq to 15 μSv/MBq, from 3 μSv/MBq to 10 μSv/MBq, from 3 μSv/MBq to 7.5 μSv/MBq, from 3 μSv/MBq to 5 μSv/MBq, from 5 μSv/MBq to 20 μSv/MBq, from 5 μSv/MBq to 15 μSv/MBq, from 5 μSv/MBq to 10 μSv/MBq, from 5 μSv/MBq to 7.5 μSv/MBq, from 7.5 μSv/MBq to 20 μSv/MBq, from 7.5 μSv/MBq to 15 μSv/MBq, from 7.5 μSv/MBq to 10 μSv/MBq, from 10 μSv/MBq to 20 μSv/MBq, from 10 μSv/MBq to 15 μSv/MBq, or from 15 μSv/MBq to 20 μSv/MBq.

In certain embodiments, the effective dose per MBq of administered pharmaceutical composition is from 5 μSv/MBq to 10 μSv/MBq, e.g., from 5 μSv/MBq to 9 μSv/MBq, from 5 μSv/MBq to 8 μSv/MBq, from 5 μSv/MBq to 7 μSv/MBq, or from 5 μSv/MBq to 6 μSv/MBq.

In certain embodiments, after administering to the subject a pharmaceutical composition disclosed herein having an activity of 100-300 MBq (e.g., 100-200 MBq. 100-150 MBq. 150-300 MBq. 150-200 MBq. 200-300 MBq, or 250-300 MBq), the absorbed dose in the pancreas is 1 Gy or less, e.g., 0.5 Gy or less, 0.3 Gy or less, 0.2 Gy or less, 0.1 Gy or less, 0.05 Gy or less, 0.02 Gy or less, or 0.01 Gy or less.

The methods of the present disclosure include a step of generating one or more radiographic images of the subject. The images can be of a region, body part, or organ of the subject. In certain embodiments, the one or more images are generated of the skull to mid-thigh of the subject. In certain embodiments, the one or more images are generated of the head and neck of the subject. In certain embodiments, the one or more images are generated of the whole body of the subject. Exemplary organs include, but are not limited to, the pituitary gland, salivary glands, thyroid gland, lungs, liver, spleen, pancreas, small intestine, adrenal glands, kidney, urinary bladder, bone marrow, upper thoracic spine, and 3rd to 5th lumbar vertebrae.

The radiographic images can be generated using positron emission tomography (PET), PET-computer tomography (PET-CT), or single-photon emission computerized tomography (SPECT). In certain embodiments, the one or more radiographic images are generated using PET. In certain embodiments, the one or more radiographic images are generated using PET-CT. In certain embodiments, the one or more radiographic images are generated using SPECT.

In certain embodiments, the one or more radiographic images of the subject are generated dynamically, i.e., concurrently with administration of the pharmaceutical composition.

In certain embodiments, the one or more radiographic images are generated after administration of the pharmaceutical composition. In certain embodiments, the one or more radiographic images are generated at least 30 minutes after administration of the pharmaceutical composition, e.g., at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, or at least 24 hours after administration of the pharmaceutical composition.

In certain embodiments, the one or more radiographic images are generated from 30 minutes to 24 hours after administration of the pharmaceutical composition, e.g., from 30 minutes to 18 hours, from 30 minutes to 10 hours, from 30 minutes to 5 hours, from 30 minutes to 3 hours, from 30 minutes to 1 hour, from 1 hour to 24 hours, from 1 hour to 18 hours, from 1 hour to 10 hours, from 1 hour to 5 hours, from 1 hour to 3 hours, from 3 hours to 24 hours, from 3 hours to 18 hours, from 3 hours to 10 hours, from 3 hours to 5 hours, from 5 hours to 24 hours, from 5 hours to 18 hours, from 5 hours to 10 hours, from 10 hours to 24 hours, from 10 hours to 18 hours, or from 18 hours to 24 hours after administration of the pharmaceutical composition.

In certain embodiments, the one or more radiographic images are generated 1 hour after administration of the pharmaceutical composition. In certain embodiments, the one or more radiographic images are generated 3 hours after administration of the pharmaceutical composition. In certain embodiments, the one or more radiographic images are generated 18 hours after administration of the pharmaceutical composition. In certain embodiments, the one or more radiographic images are generated 24 hours after administration of the pharmaceutical composition.

In certain embodiments, the one or more radiographic images are generated 1 hour and 3 hours after administration of the pharmaceutical composition. In certain embodiments, the one or more radiographic images are generated 1 hour and 18 hours after administration of the pharmaceutical composition. In certain embodiments, the one or more radiographic images are generated 1 hour and 24 hours after administration of the pharmaceutical composition. In certain embodiments, the one or more radiographic images are generated 1 hour, 3 hours, and 18 hours after administration of the pharmaceutical composition. In certain embodiments, the one or more radiographic images are generated 1 hour, 3 hours, 18 hours, and 24 hours after administration of the pharmaceutical composition.

In certain embodiments, the one or more radiographic images are generated 3 hours and 18 hours after administration of the pharmaceutical composition. In certain embodiments, the one or more radiographic images are generated 3 hours and 24 hours after administration of the pharmaceutical composition. In certain embodiments, the one or more radiographic images are generated 3 hours, 18 hours, and 24 hours after administration of the pharmaceutical composition.

In certain embodiments, the one or more radiographic images are generated 18 and 24 hours after administration of the pharmaceutical composition.

In certain embodiments, the method further comprises determining one or more of the following from the radiographic images: maximum standardized uptake volume (SUVmax), tumor detection rate (TDR), differential tumor detection rate (DDR), signal-to-noise ratio (SNR=SUVmax in tumor volume of interest (VOI)), tumor-to-background ratio(s) (TBR), sensitivity, and positivity predictive value (PPV).

In certain embodiments, the methods of the present disclosure have a sensitivity that is at least 15% greater than using a standard of care method (e.g., a method utilizing ⁶⁸Ga-DOTA-TOC), e.g., at least 25%, at least 50%, at least 75%, at least 100%, at least 125%, at least 150%, at least 175%, or at least 200%.

In certain embodiments, the methods of the present disclosure have a positive predictive value (PPV) that is at least 15% greater than using a standard of care method (e.g., a method utilizing ⁶⁸Ga-DOTA-TOC), e.g., at least 25%, at least 50%, at least 75%, at least 100%, at least 125%, at least 150%, at least 175%, or at least 200%.

In certain embodiments, following generating one or more images, localization (absolute) and amount of localization of the radionuclide in the one or more radiographic images of the subject can be determined, e.g., by medical personnel.

Based on the localization of the radionuclide, one or more of the following can occur: staging or restaging the subject's cancer, selecting the subject for PRRT, determining the subject's cancer treatment protocol, and determining the subject's response to cancer treatment.

In certain embodiments, reading of the one or more radiographic images generated by the methods described herein enables staging the subject's cancer. In certain embodiments, reading of the one or more radiographic images generated by the methods described herein enables restaging the subject's cancer. A number of staging methods are known in the art, e.g., the “TNM system,”, where “T” is the primary tumor (T category), “N” is lymph nodes (N category” and “M” is metastasis (M category). In certain embodiments, a cancer's grade is included in the stage. Once the values for T, N, and M (and any other factors that affect stage) have been determined, they are combined to assign an overall stage. For most cancers, the stage is a Roman numeral from I (1) to IV (4). Stage I cancers are less advanced and often have a better prognosis (outlook). Higher stage cancers typically have spread farther (or have other concerning features) and might require more intense (or different kinds of) treatment. Sometimes stages are subdivided as well, using capital letters (for example, stage III might be subdivided into stages IIIA and IIIB). Some cancers also have a stage 0, which is often called carcinoma in situ. This means the cancer is still only in the layer of cells where it first started, and it has not spread any farther.

In certain embodiments, reading of the one or more radiographic images generated by the methods described herein enables selection of the subject for PRRT.

In certain embodiments, reading of the one or more radiographic images generated by the methods described herein enables determination of the subject's cancer treatment protocol, e.g., surgery, chemotherapy, drug therapy, PRRT, gene therapy, cryotherapy, or a combination thereof.

In certain embodiments, reading of the one or more radiographic images generated by the methods described herein enables determination of the subject's response to cancer treatment, e.g., responding to treatment or not responding to treatment. In such embodiments, the pharmaceutical composition is administered to the subject at an earlier time point and at a later time point, and one or more images are generated at both the earlier time point and the later time point. The images generated at the earlier time point and later time point are compared. Subjects responding to treatment have decreased radiotracer localization at the later imaging time point compared to the earlier imaging timepoint.

In certain embodiments, the earlier time point is before the subject begins cancer treatment. In certain embodiments, the earlier time point is at least 1 day before the subject begins cancer treatment, e.g., at least 1 week, at least 2 weeks, at least 3 weeks, or at least 1 month before the subject begins cancer treatment.

In certain embodiments, the earlier time point is after the subject begins cancer treatment. In certain embodiments, the earlier time point is at least 1 week after the subject begins cancer treatment, e.g., at least 2 weeks, at least 1 month, at least 2 months, at least 3 months, at least 6 months, at least 9 months, or at least 12 months after the subject begins cancer treatment.

In certain embodiments, the later time point is at least 1 day after the earlier time point, e.g., at least 2 days, at least 3 days, at least 4 days, at least 5 days, at least 6 days, at least 1 week, at least 2 weeks, at least 3 weeks, at least 4 weeks, at least 2 months, at least 3 months, at least 6 months, at least 9 months, or at least 1 year after the earlier time point.

In certain embodiments, the later time point is after the subject finishes cancer treatment. In certain embodiments, the later time point is at least 1 week after the subject finishes cancer treatment, e.g., at least 2 weeks, at least 1 month, at least 2 months, at least 3 months, at least 6 months, at least 9 months, or at least 12 months after the subject finishes cancer treatment.

In certain embodiments, the earlier time point is before the subject begins cancer treatment and the later time point is after the subject finishes cancer treatment (i.e., to determine the effectiveness of a previous treatment). In certain embodiments, the earlier time point is before the subject begins cancer treatment and the later time point is after the subject begins cancer treatment (i.e., to determine the effectiveness of the ongoing treatment).

In certain embodiments, following generation of the one or more radiographic images, the subject is then treated with a second pharmaceutical composition comprising a radiopharmaceutical (i.e., theranostics). In certain embodiments, the radiopharmaceutical is selected from [⁶⁷Cu]Cu-NODAGA-LM3, [177Lu]Lu-DOTA-LM3, [177Yb]Yb-DOTA-LM3, [²²⁵Ac]Ac-DOTA-LM3, [212Pb]Pb-DOTA-LM3, [177Lu]Lu-DOTA-TATE, [¹⁷⁷Yb]Yb-DOTA-TATE, [²²⁵Ac]Ac-DOTA-TATE, [²¹²Pb]Pb-DOTA-LM3-DOTA-TATE, [¹⁷⁷Lu]Lu-DOTA-JR11 (OPS201), [⁹⁰Y]Y-DOTA-JR11, [225 Ac]Ac-DOTA-JR11, [177Lu]Lu-DOTA-TOC, [⁹⁰Y]Y-DOTA-TOC, [²²⁵Ac]Ac-DOTA-TOC, 213Bi-DOTA-TOC, [¹⁷⁷Lu]Lu-DOTA-EB-TATE, [¹⁷⁷Lu]Lu-DOTA-LNC1010, [212Pb]Pb-DOTA-LM3-DOTAMTATE, [⁹⁰Y]Y-DOTALAN, and [²¹²Pb]Pb-VMT-α-NET.

In another aspect, the present disclosure provides theranostic methods comprising administering to a subject an effective amount of a first composition corresponding to a pharmaceutical composition described hereinabove comprising a radiotracer that is ⁶¹Cu-NODAGA-LM3 or is a pharmaceutically acceptable salt thereof, generating one or more images of the subject, and administering to the subject an effective amount of a second pharmaceutical composition comprising a therapeutic radiopharmaceutical. Administration of the first composition corresponding to the pharmaceutical composition described herein and generation of the one or more images is carried out as set forth above. In certain embodiments, radiopharmaceutical of the second composition is selected from [⁶⁷Cu]Cu-NODAGA-LM3, [177Lu]Lu-DOTA-LM3, [177Yb]Yb-DOTA-LM3, [225 Ac]Ac-DOTA-LM3, [212Pb]Pb-DOTA-LM3, [177Lu]Lu-DOTA-TATE, [¹⁷⁷Yb]Yb-DOTA-TATE, [²²⁵Ac]Ac-DOTA-TATE, [²¹²Pb]Pb-DOTA-LM3-DOTA-TATE, [¹⁷⁷Lu]Lu-DOTA-JR11 (OPS201), [⁹⁰Y]Y-DOTA-JR11, [225 Ac]Ac-DOTA-JR11, [177Lu]Lu-DOTA-TOC, [′0Y]Y-DOTA-TOC, [²²⁵Ac]Ac-DOTA-TOC, 213Bi-DOTA-TOC, [¹⁷⁷Lu]Lu-DOTA-EB-TATE, [¹⁷⁷Lu]Lu-DOTA-LNC1010, [212Pb]Pb-DOTA-LM3-DOTAMTATE, [⁹⁰Y]Y-DOTALAN, and [²¹²Pb]Pb-VMT-α-NET.

### 5. EXAMPLES

### 5.1. Example 1: [⁶¹Cu]CuCl₂Production

The production of the radionuclide was performed by irradiating a solid target of highly pure ⁶¹Ni (purity ≥99.42%) plated on a niobium coin backing with a proton beam.

Due to the relatively short half-lives (t_{1/2} ⁶⁸Ga=68 min; ¹⁸F=110 min) and physical properties of the radionuclides, the key challenges in the PET tracer industry remain a) the imaging quality, b) reliability of supply and distribution of the radiopharmaceutical at low cost and c) low radiation burden to the patient. The distinctive advantage of using ⁶¹Cu as a positron emitter, e.g., in a PET tracer, will not only ensure a) good imaging quality due to its physical properties (low mean positron energy) but also the possibility of delayed imaging, expected to improve the diagnostic sensitivity due to the washout of radioactivity from the background, thus improved image contrast, b) a large distribution radius due to its relatively long half-life (t_{1/2} ⁶¹Cu=205.5 min) while c) still keeping the radiation burden to the patient at a minimum. Provided herewith is an enabling description of new processes to produce highly pure ⁶¹Cu, in the form of [⁶¹Cu]CuCl₂, to be used in radiopharmaceutical applications, e.g., as a positron emitter in a PET tracer, in high activity concentration and volumes.

Trace metals and cold copper compete with ⁶¹Cu to bind a chelator (for example, NODAGA) in this order: cold Cu(II) (i.e., stable isotopes)>Zn(II)>Fe(III)>Sn(IV)>Ti(IV)>Al(III.). The competition from these trace metals and cold copper decreases the tracer's radiolabeling yield and radiochemical purity significantly. Frequent sources of trace metals are the raw nickel metal powder itself, especially isotopically enriched nickel, reagents, and any metals in instruments used, such as iron. The purification process (ion-exchange columns) removes much of the trace metals except for cold (of particular relevance are stable isotopes 69Cu and 65Cu), which passes through into the product fraction by being the same element as the desired ⁶¹Cu. One way of preventing cold copper contamination and the associated reduction in chemical purity is to pass the dissolved nickel raw material (stable isotopes) through the process and separate the cold copper from the nickel before plating (see FIG. 8 for ICP-MS analysis). Table 1 displays the chemical purity of the [⁶¹Cu]CuCl₂ by either bombardment of ^{nat}Ni or ⁶¹Ni on a niobium backing and the resulting impurity profile.

TABLE 1
Chemical Purity of ⁶¹Cu transmuted from ^{nat}Ni vs. ⁶¹Ni.

ng/MBq

| | Cu-61 from nat-Ni | Cu-61 from Ni-61 |
|---|---|---|
| Aluminum (Al) | 1.1 | 0.3 |
| Cobalt (Co) | N/D | 0.2 |
| Copper (Cu) | 0.3 | 0.6 |
| Iron (Fe) | 1.6 | 1.5 |
| Lead (Pb) | 0.4 | 0.7 |
| Nickel (Ni) | 3.4 | 0.1 |
| Zinc (Zn) | 0.7 | 0.1 |

### 5.1.1. Preparation of Plating Solution

### 5.1.1.1 Preparation of Buffer Solution

Ammonium Chloride (4.6 g, Aldrich: 326372, Trace Select) was weighed into a clean (no metal) Falcon Tube (50 mL), and the previously cleaned magnetic stirring bar was added. 6 mL of Trace Select water (Honeywell 95305) was added in one aliquot to flush walls of the Falcon in case any salt sticks to the Falcon tube walls. 1 mL of ammonium hydroxide 28% (Sigma 338818) was added with a 1000 μL pipette with a respective pipette tip, 8× times. The lid of the Falcon was closed, and the Falcon is, in turns, vortexed (1-2 minutes) (immersion in an ultra-sonic bath was a possible alternative for 1-2 minutes) and shaken, until all salt was dissolved. The Falcon tube can also be warmed (e.g., by rolling between hands) to improve solubility, temperature (e.g., around 23° C., preferably between 23-25° C.). After complete dissolution of the salt, the pH acceptance criteria, pH range 9.28-9.62, was verified by pH measurement of the solution at RT, e.g., with and electronic pH meter. The Falcon tube was closed with parafilm and stored at room temperature. Prior to use, any solid salt formation was redissolved.

### 5.1.1.2 Preparation of Nickel Nitrate Plating Solution

A 50 mL glass beaker was washed with nitric acid (Trace Select) followed by water (Trace Select). In a fume hood, the beaker was dried by placing it on a heating plate set to 150° C. To the beaker was added 210 μg of natural (isotopic distribution) nickel (powder, Sigma-Aldrich ≤50 μm, 99.7% trace metals basis, essentially free from any impurities, except iron. The copper impurity amounts to ≤0.3 ppm.) were weighed into the beaker and 4 mL of 65% nitric acid were added using a pipette. The beaker was placed back on the active heating plate and the stirring was set to 300 rpm. Ensure the ventilation of the fume hood was functioning properly (evolution of NO₂). During the dissolution, the solution turned green. The solution was reduced by evaporation to a volume of ≈600 UL and taken from the heating plate to cool down to room temperature. The remaining solution was transferred to a 50 mL metal-free Falcon tube. The glass beaker was rinsed with a total of 2.8 mL of Trace Select water, in steps of 0.8 mL, 1 mL, and 1 mL, where each step was transferred to the Falcon tube before the adding the next washing fraction. Buffer solution (4 mL), 11 mL of Trace Select water, and 3 mL of ammonium hydroxide 28% (Sigma 338818) were added to the Falcon tube. The pH of the solution was measured and adjusted to the required pH by adding ammonium hydroxide 28% (Aldrich 338818) using sterile B-Braun syringes.

The following are example lots of ⁶⁰Ni and ⁶¹Ni (certificate as provided by Isoflex, USA, March 2018):

TABLE 2

| Isotope | ⁶¹Ni |
|---|---|
| Enrichment | 86.20% |
| Form | Metal ingot/powder |
| Certificate | 6275 |

| | Isotope | Ni-58 | Ni-60 | Ni-61 | Ni-62 | Ni-64 |
|---|---|---|---|---|---|---|
| Isotopic | Content (%) | 1.17 | 0.8 | 86.2 | 11.7 | 0.14 |
| distribution | | | | | | |

| | Element | Al | Bi | Ca | Cd | Co | Cr | Cu | Fe | K | μg |
|---|---|---|---|---|---|---|---|---|---|---|---|
| Chemical | Content | 10 | <10 | 20 | 10 | <10 | <10 | 20 | 40 | <10 | <50 |
| admixtures | (ppm) | | | | | | | | | | |

| | Element | Mo | Mn | Na | Pb | Si | Sn | Zn |
|---|---|---|---|---|---|---|---|---|
| Chemical | Content | <8 | <50 | <10 | <10 | 20 | 30 | 50 |
| admixtures | (ppm) | | | | | | | |

TABLE 3

| | |
|---|---|
| Isotope | ⁶¹Ni |
| Enrichment | 99.39% |
| Form | Metal powder |
| Certificate | TBD/not specified |

| | Isotope | Ni-58 | Ni-60 | Ni-61 | Ni-62 | Ni-64 |
|---|---|---|---|---|---|---|
| Isotopic | Content | 0.01 | 0.29 | 99.39 | 0.29 | 0.02 |
| distribution | (%) | | | | | |

| | Element | Al | Co | Cr | Cu | Fe | μg | Mn | Pb | Si | Ti |
|---|---|---|---|---|---|---|---|---|---|---|---|
| Chemical | Content | 12 | <10 | <10 | 14 | <10 | <10 | <10 | <10 | <10 | <10 |
| admixtures | (ppm) | | | | | | | | | | |

| | Element | Zn | C | S |
|---|---|---|---|---|
| Chemical | Content | <10 | 157 | <10 |
| admixtures | (ppm) | | | |

TABLE 4

| | |
|---|---|
| Isotope | ⁶⁰Ni |
| Enrichment | 99.31% |
| Form | metal powder |
| Certificate | TBD/not specified |

| | Isotope | Ni-58 | Ni-60 | Ni-61 | Ni-62 | Ni-64 |
|---|---|---|---|---|---|---|
| Isotopic | Content | 0.21 | 99.31 | 0.46 | 0.015 | 0.005 |
| distribution | (%) | | | | | |

| | Element | Al | Co | Cr | Cu | Fe | μg | Mn | Pb | Si | Ti |
|---|---|---|---|---|---|---|---|---|---|---|---|
| Chemical | Content | <10 | 70 | 20 | 25 | <10 | <10 | <10 | <10 | 15 | <10 |
| admixtures | (ppm) | | | | | | | | | | |

| | Element | Zn | C | S | P |
|---|---|---|---|---|---|
| Chemical | Content | 15 | 114 | 20 | 30 |
| admixtures | (ppm) | | | | |

The samples of natural nickel from Sigma-Aldrich were essentially free from any impurities, except iron. The copper impurity amounts to ≤0.3 ppm. Additional suitable sources of natural Ni include:
Nickel powder, ≤50 μm, 99.7% trace metals basis
Nickel rod, diam. 6.35 mm, =99.99% trace metals basis
Nickel foil, thickness 0.5 mm, 99.98% trace metals

### 5.1.2. Electroplating the Backing Surface

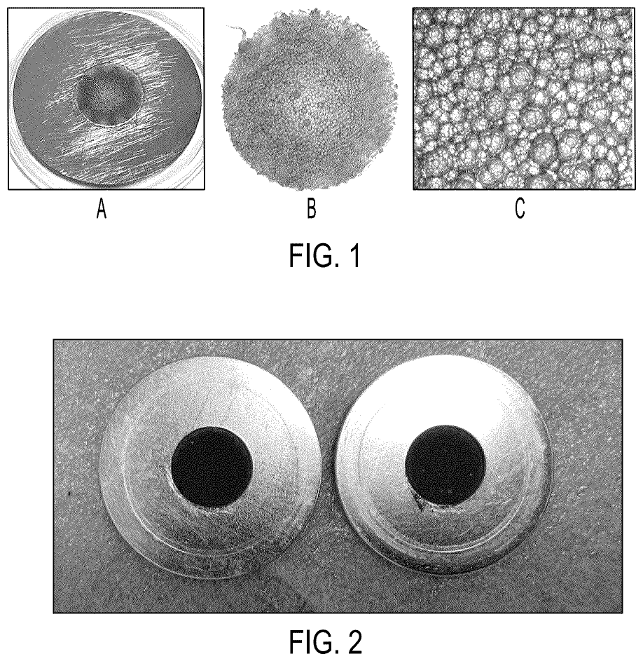

A disc shaped niobium backing was obtained from high purity Nb as described herein and (28 mm×1.0 mm) was cleaned with ethanol (high-purity) and inserted in a Comecer Electroplating Unit V21204. A platinum wire anode was positioned so that the distance relative to the coin surface was between about 1 and 3 mm, adjusted by a polymer spacer. The coin mass was determined to be 5.25 grams. Niobium backing (22 mm×1.0 mm weighs 3.3 g). The plating solution was charged to the electrolyte container and attached to the apparatus. The voltage was set to 4.5V. The current reading after 5 min stabilization was 180 μA. The duty cycle for pump was set to 45%. The plating liquid turned from blue to transparent, slow decrease of current to 160 μA was observed over the period of 120 minutes. The plating process was stopped. The coin was taken out of the electrolytic cell and its weight was measured. The coin also underwent microscopic evaluation, FIGS. 1 and 2 using a DINOLite digital microscope to observe the crystal structure and homogeneity of the surface. The coin (FIG. 2) was stored in a metal-free Falcon tube under a nitrogen atmosphere.

### 5.1.3. Results of the Electroplating

Upon completion of electroplating, the coin underwent a microscopic evaluation using a DINOLite digital microscope to observe the crystal structure and homogeneity of the surface. As can be seen in FIG. 1 (panels A-C), a homogenous target coating having durable adhesion was obtained, see also FIG. 2.

5.1.4. General Guidelines for High-purity [⁶¹Cu]Cl₂ Production

The purpose of this example was to enable the bulk production of ⁶¹Cu from the deuteron irradiation of natural nickel and/or enriched ⁶⁰Ni. This effort was a proof of concept, and, therefore, there were no benchmarked specifications for ⁶¹Cu. However, we optimized target performance, target geometry/material use, irradiation parameters, and chemical processing methods to produce [⁶¹Cu]CuCl₂ following enriched ⁶⁰Ni irradiation, or, scaled accordingly for ^{nat}Ni irradiation. There were no pharmacopoeia specifications for radio-copper explicitly, however, test QC methods include assessment of radionuclidic purity and molar activity (to demonstrate usability of the extracted [⁶¹Cu]CuCl₂).

This example considers use of two different types of targets, natural nickel (^{nat}Ni) targets and highly enriched Nickel-60 (⁶⁰Ni) targets both of which were suitable for deuteron bombardment. However, ^{nat}Ni was cheap and available in high-purity while ⁶⁰Ni was still costly and required efficiency measures. If even higher yields were desired, target preparation efforts may be directly translated into the proton-based ⁶¹Ni(p,n)⁶¹Cu route, however, given the cost of enriched ⁶¹Ni (c.a. $25 USD/μg), such an approach imposes the need for target recycling.

The set of guidelines below enable all types of targets in the production of ⁶¹Cu, including the production of high-purity [⁶¹Cu]CuCl₂ from the Nb coins with a Zn or Ni (any isotopic enrichment) coating electroplated thereon as provided herein. Specific details are also provided for deuteron, and proton irradiations, respectively. This protocol was followed to generate the ⁶¹Cu-compositions evaluated in the following examples.

| Target Backing | Flat coin - disc-shaped. The dimensions of the target backing form are: | |
|---|---|---|
| Geometry | Coin backing: | |
| | Diameter Ø = 20-30 ± 0.1 mm | |
| | Thickness H = 1.5 mm | |
| | Target Ni layer or coating | |
| | a. Diameter 13 mm (deuteron) or 10 mm (proton) | |
| | b. Mass 70-100 mg, e.g., around 100 ± 40 mg (deuteron) or | |
| | around 50 ± 20 mg (proton) | |
| | c. Thickness (H) full density (d = 8.9) | |
| | i. Hₘᵢₙ = 0.1 mm; Hₘₐₓ = 0.14 mm corresponding to | |
| | 70-100 mg deposited | |
| | Tolerances/finishes unless otherwise stated are as follows: | |
| | Surface finish: Ra 1.6 | |
| | General tolerance: ISO 2768-m | |
| | Sharp edges and corners according to ISO 13 715 | |
| Target Backing | Optional - Surface treated with abrasion by pink corundum grindstone - | |
| Surface | free of impurities | |
| Target Backing | Niobium foil, 99.8% (metals basis), 1.0 mm (0.04 in) thick, annealed, | |
| Material | Stock No.: 10257 | |
| | Lot No.: C15P07 | |
| | Element | ppm |
| | Carbon | 24 |
| | Hydrogen | 1 |
| | Molybdenum | 2 |
| | Nickel | 4 |
| | Silicon | 1 |
| | Titanium | 2 |
| | Zirconium | 3 |
| | Iron | 1 |
| | Hafnium | 2 |
| | Nitrogen | 14 |
| | Oxygen | 56 |
| | Tantalum | 785 |
| | Tungsten | 4 |
| Target Backing | Niobium 99.9% typical certificate of analysis results, Goodfellows | |
| Material | Product nr. 931-627-20 | |
| | Element | ppm |
| | B | <10 ppm |
| | Ni | <5 ppm |
| | O | 100 ppm |
| | Si | 100 ppm |
| | Zr | <10 ppm |
| | Ta | 500 ppm |
| | H | <10 ppm |
| | W | <100 ppm |
| | C | 25 ppm |
| | N | 20 ppm |
| | Fe | 30 ppm |
| | Cu | <5 ppm |
| | Mo | 10 ppm |
| | Ti | <10 ppm |

| | |
|---|---|
| Transfer system | As the target can be automatically transferred to/from the cyclotron by |
| compatibility | means of a pneumatic target transfer system, it was critical that the |
| | deposited Ni was robust to direct air flow and abrupt mechanical |
| | movements. |
| | In certain embodiments, the target coating remains adhered to the backing |
| | during pneumatic transfer both to and from the cyclotron. Such a pneumatic |
| | system was typically fed by a compressed air connection of ~6-7 bar, and at |
| | minimum, 360 SLPM flow. Such a system was “push-push”, and therefore, |
| | compressed air was typically blown on both the front and rear sides of the |
| | coin, respectively, depending on the direction of transfer. The coin will also |
| | come to an abrupt stop as it reaches the target station or hot cell. |
| | In certain embodiments, suitable tests that indicate target durability include |
| | the following, whereby the total mass loss for all tests combined should be |
| | negligible (e.g. <1 μg): Visual inspection, gentle knocking/tapping on a |
| | countertop on top of white paper to check for loosening of target coating |
| | grains, gently rubbing an acid-washed Teflon spatula against the deposited |
| | target coating and checking for loosening of target coating grains, and/or |
| | placing and gently pressing down on a piece of Scotch tape against the |
| | target coating. |
| | If there was access to the cyclotron apparatus, it was recommended to |
| | transfer the coin back/forth multiple times and ensure target coating |
| | stability (i.e., no mass loss). Such a test may be performed with a degrader |
| | in place. |
| Method of | Electrodeposition from bath with a significantly high pH (e.g., 9.9-10.8) |
| Production | |
| Target Metal | To withstand the deposited beam power, the target metal was preferably |
| Form | metallic nickel (not, e.g., nickel oxide). |
| | Depending on the means of target preparation (e.g. electroplating), the raw |
| | nickel starting material need not necessarily be metallic. However, methods |
| | used for preparing ^{nat}Ni targets should ultimately be directly translatable to |
| | preparation of ⁶⁰Ni or ⁶¹Ni targets. At present, it was understood that |
| | enriched Ni was typically in the form of a salt. |
| Target Additives | The use of binders must not necessary be avoided if they are absent of the |
| | final metallic coin and if an assessment on a case-by-case basis to |
| | understand potential impact to product quality has been done (e.g. ICP-MS |
| | on the binder material. |
| | Any reagents used for target preparation (e.g. electroplating reagents) must |
| | be of the highest quality, in particular, with regards to trace metals. |
| Metal Content | Preferably, the highest grades of reagents should be used, to avoid trace |
| | metals contamination of the target coating, as more than a tenth of a |
| | microgram per 100 μg of target metal (that is, 1 ppm of the target metal) is |
| | already a significant contamination that may render the coin unusable for |
| | production of high-purity radionuclides. In the case of the production of |
| | radiocopper it is not accepted to add more than 0.1 ppm of cold Cu as this |
| | would reduce the purity of the prepared radionuclide composition. |
| | Preferably, max level of impurities allowed to be added by the process to |
| | the initial nickel: |
| | Copper (Cu): 0.1 ppm |
| | High affinity metals (Ga, Lu, Pb, Y): 0.1 ppm |
| | Zinc and cobalt (Zn, Co): 0.3 ppm |
| | Transition and other metals (Cd, Cr, Al, Mn, Mo, Sn, Ti, V . . . ): 1 |
| | ppm on a case by case |
| | Iron (Fe): 10 ppm |
| | Family I and II (K, Ba, μg, Be . . . ): 1000 ppm |
| | The metal coins were analyzed on a batch per batch basis by dissolution in |
| | nitric acid to assess the metal contamination within the coin that were not |
| | found in the starting nickel metal and thus originate from the process. |
| | The amount suggested above were a good, albeit not strict, guide since |
| | chemical purification following irradiation will, in turn, further remove |
| | some of these impurities. The ultimate specification on this front will |
| | therefore be an iterative process as the Cu/Ni separation chemistry is |
| | refined. However, the process shall not significantly add impurities that |
| | were not in the originating pure nickel material. |
| | It is preferable that cold Cu should be minimized in the deposited Ni since |
| | this will follow the chemistry of any ⁶¹Cu and cannot be separated post- |
| | irradiation. Any such cold Cu will directly compete with ⁶¹Cu during |
| | radiolabeling of the pharmaceutical. Methods of removing Cu from the |
| | dissolved target metal are well known. |
| Density of Target | To withstand the deposited beam power, the Ni target should preferably be |
| | of reasonably high volumetric density (e.g. approximately ≥90% or, ≥8.0 |
| | g/cm³). |
| Power Rating | The power rating for the target, including the combined deposited Ni and |
| | plate should preferably be: |
| | ≥420 W (deuterons) |
| | ≥820 W (protons) |
| Loading Mass of | The loading mass vs. the deposited mass of Ni (i.e. deposition efficiency) |
| Target | relates not to technical specifications, but rather, to cost. In the case of ^{nat}Ni |
| | deposition, loading efficiency will not have a significant impact on the cost |
| | of ⁶¹Cu. However, losses should be minimized in considering the translation |
| | to enriched ^{6x}Ni. For ⁶⁰Ni, losses should be maintained below ~10%, and |
| | for ⁶¹Ni, below ~1%. Some techniques such as magnetron sputtering are |
| | thus not possible for enriched nickel but are satisfactory for ^{nat}Ni. |
| Mass/thickness | For deuterons (i.e. ^{nat}Ni or ⁶⁰Ni), the thickness should be appropriate for |
| of Nickel | stopping the deuterons, with a maximum 10% variability in material |
| | deposition. Such thicknesses equate to: |
| | ≥100 μm (assuming 100% density) |
| | ≥70 mg or ≥89 mg/cm² (assuming 10 mm diameter) |
| | For protons (i.e. ⁶¹Ni), one may wish to selectively limit the deposited |
| | material to optimize the balance between material cost, yield, and backing |
| | material activation. With a maximum 10% variability in material |
| | deposition, four examples are noted below. |
| | ⁶¹Ni Scenario #1 (11→9 MeV) |
| | 78 μm (assuming 100% density) |
| | 55 mg or 69 mg/cm² (assuming 10 mm diameter well) |
| | ⁶¹Ni Scenario #1 (12→8 MeV) |
| | 155 μm (assuming 100% density) |
| | 108 mg or 138 mg/cm² (assuming 10 mm diameter well) |
| | ⁶¹Ni Scenario #1 (13→7 MeV) |
| | 233 μm (assuming 100% density) |
| | 163 mg or 208 mg/cm² (assuming 10 mm diameter well) |
| | ⁶¹Ni Scenario #1 (13→4 MeV) |
| | 309 μm (assuming 100% density) |
| | 216 mg or 275 mg/cm² (assuming 10 mm diameter well) |
| Isotopic | The ^{6x}Cu radioisotopes which will be coproduced during production of ⁶¹Cu |
| enrichment | (t ½ = 3.339 h) include: |
| | ⁵⁷Cu (t ½ = 0.196 s) ⁵⁸Cu (t ½ = 3.204 s) |
| | ⁵⁹Cu (t ½ = 81.5 s) ⁶⁰Cu (t ½ = 23.7 m) |
| | ⁶²Cu (t ½ = 9.673 m) ⁶⁴Cu (t ½ = 12.701 h) |
| | From a practical handling point of view, all but ⁶⁰Cu and ⁶⁴Cu are likely to |
| | have decayed prior to use. Only ⁶⁴Cu will have any impact on the possible |
| | shelf-life of ⁶¹Cu. |
| | In addition to the production of Cu radioisotopes, other radionuclides (e.g. |
| | Co and Ni) will also be produced, the ratio of which will depend on the |
| | isotopic composition, and whether undergoing deuteron or proton |
| | irradiation. As these byproducts are chemically different from copper, such |
| | radionuclides may be removed during ⁶¹Cu purification/processing. For |
| | example, The Cu-61 was purified from metal and radiometal impurities via |
| | a GE Healthcare FASTlab 2 module through a tributyl phosphate resin |
| | cartridge and a tertiary-amine-based weak ionic exchange resin containing |
| | long-chained alcohols. |
| Any other | Niobium is preferred over silver for its better resistance to corrosion, its low |
| requirements | amount of activation on irradiation and for its high melting temperature that |
| | permits the deposit of nickel by other processes such as melting or heat |
| | sintering. However, silver possesses a higher thermal conductivity and may |
| | be suitable for certain embodiments. |
| | For target backing manufacture, the following sheet of niobium is suitable |
| | for laser cutting: |
| | http://www.Goodfellow.com |
| | NB000400 Niobium Foil, Size: 150 × 150 mm Thickness: 1.5 mm, |
| | Purity: 99.9%, Temper: Annealed, Quality: LT |
| | From one sheet up to 25 target backings can be manufactured. |

5.1.5. Purification and Characterization of [⁶¹Cu]CuCl₂ and Waste Streams

The solid target irradiated material was dissolved in a total volume of 7 mL of 6 M HCl with the addition of 30% hydrogen peroxide via a dissolution chamber. Separation and purification was accomplished using a cassette-based FASTlab platform using a TBP (tributylphosphate-based) resin (1 mL) (particle size 50-100 μm; pre-packed, Triskem) then a weakly basic (tertiary amine; TK201) resin (2 mL) (particle size 50-100 μm; pre-packed, Triskem), each of which were pre-conditioned with H₂O (7 mL) and HCl (10M, 7 mL). The cassette reagent vials were prepared using concentrated HCl (Optima Grade, Fischer Scientific), NaCl (ACS, Fischer Scientific) and milli-Q water (Millipore system, 18 MΩ-cm resistivity). 6M HCl (2×4.2 mL), 5M NaCl in 0.05 M HCl (4.2 mL). The subsequent ⁶¹Cu was then purified with two subsequent ion exchange resins in a FASTlab synthesis unit.

1) The acid-adjusted dissolution solution (approx. 7 mL) was loaded over both columns in series and directed into a “Ni collection fraction”. The TBP resin acted as a guard column as it quantitatively retained Fe³⁺ ions, while the Cu²⁺ and Co²⁺ complexes were quantitatively retained on the tertiary amine (TK201) resin.

2) Both columns were washed with 6M HCl (4 mL) to maximize Ni recovery for future recycling.

3) The TK201 column was washed with 4.5M HCl (5.5 mL) to elute the majority of cobalt salts.

4) The TK201 column was washed with 5M NaCl in 0.05M HCl (4 mL) to decrease residual acid on the resin and further remove any residual cobalt salts.

5) The TK201 column was washed with of 0.05M HCl (3 mL) to quantitatively elute the [⁶¹Cu]CuCl₂.

The resulting [⁶¹Cu]CuCl₂ solution of the plated material has an average activity of 1.0-4.5 GBq. This activity was measured using a dose calibrator from Comecer and its radionuclidic purity by a gamma spectrometer at PSI in Switzerland.

Gamma spectrometry measurements were performed to identify any radionuclidic impurities, particularly long-lived radionuclides. These results indicate a 89.3% and 94% reduction in impurities for ^{nat}Ni and ⁶¹Ni on niobium backing materials with respect to silver backing materials when utilizing the methods disclosed herein. ICP-MS measurements were performed on the product of cold dissolutions by Labor Veritas in Switzerland to monitor elemental impurities present in product according to ICH-Q3D. All detected impurities were within regulated ICH-Q3D concentrations (see ICH-Q3D Guidelines, page 25).

The plating of highly enriched ⁶¹Ni was also enabled with the same plating parameters as described above, for a higher yield and industrial production using proton irradiation (typically at 80 μA to 100 μA. 13 MeV protons for 1 hour to 2 hours and up to one half-life of ⁶¹Cu).

5.1.6. Purity and Activity Evaluations of [⁶¹Cu]CuCl₂ Compositions Prepared from ^{nat}Ni(d,n)⁶¹Cu and ⁶⁰Ni(d,n)⁶¹Cu Using Nb-Backed Coins.

This example presents information on the activity of the produced ⁶¹Cu generated using the Nb backing, Ni electrodeposited coins of the present disclosure; alongside cobalt radioisotopes, that were produced with deuteron irradiation using the coin comprising a natural nickel target and the coin comprising enriched ⁶⁰Ni as target, i.e., ^{nat}Ni(d,n)⁶¹Cu and ⁶⁰Ni(d,n) ⁶¹Cu, respectively. The irradiated materials were dissolved and purified as described above.

The obtained and purified ⁶¹Cu product and waste generated during purification from the products of deuteron irradiation of natural nickel/Nb coin and ⁶⁰Ni/Nb coin, respectively, was processed and analyzed by gamma-spectrometry and presented below.

TENDL-2019 is based on thick target yield calculations using isotopic abundancy of natural nickel/Nb coin and enriched ⁶⁰Ni/Nb coin, respectively.

### 5.1.7. Radiocobalt Content

Table 5 contains activities of cobalt radioisotopes in the different fractions post FASTlab purification as a mean of three measurements (n=3 irradiations) using ^{nat}Ni/Nb target coin. The activities were extrapolated to a 3 h and 50 μA beam at EoB (end of bombardment)+2 h. The activity of [⁶¹Cu]CuCl₂ in these irradiations was determined experimentally and confirmed to be ˜80% of TENDL-2019 based estimates.

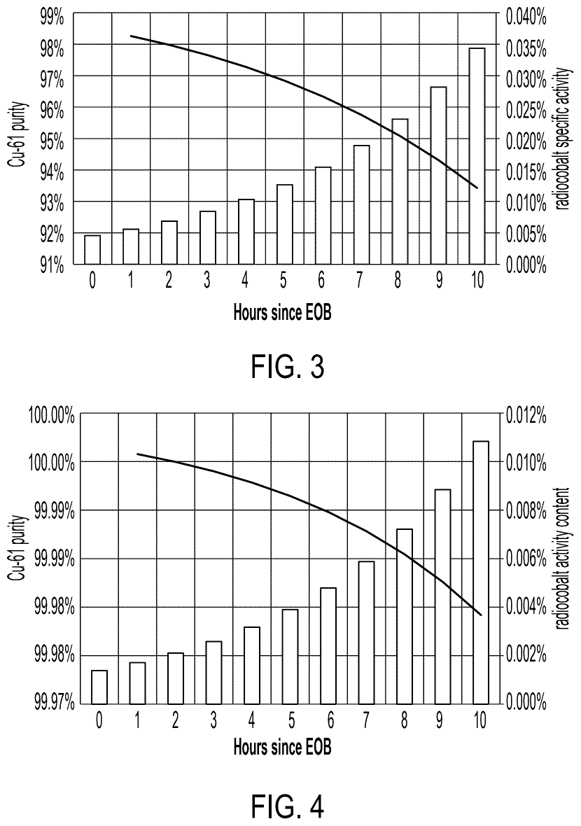

Activity of produced ⁶¹Cu for irradiation with deuteron at 8.4 MeV, 3 h at 50 μA at 80% efficiency (EoB+2 h): 3052 MBq. Also see FIG. 3 for the change in cobalt radioisotopes with time along with the corresponding change in ⁶¹Cu purity.

TABLE 5
Cobalt isotopes: natNi/Nb target coin

| Radionuclide | Cu fraction [Bq] | Ni fraction [Bq] | Co-waste I + II [Bq] | Half-life [days] |
|---|---|---|---|---|
| ⁵⁶Co | 118345 | 2696 | 2458071 | 77 |
| ⁵⁷Co | 0 | 0 | 474 | 272 |
| ⁵⁸Co | 95395 | 2145 | 1940192 | 71 |
| ⁶⁰Co | 124 | 3 | 2602 | 1925 |

Table 6 contains calculated activities of cobalt radioisotopes that would be obtained by using 99% enriched ⁶⁰Ni as target metal. The activities were extrapolated to a 3 h and 50 μA beam at EoB+2 h. The activity of ⁶¹Cu was calculated accordingly.

The activity of produced ⁶¹Cu with deuteron irradiation at 8.4 MeV, 3 h at 50 HA at 80% efficiency (EoB+2 h) was 11.552 MBq. Also see FIG. 4 for the change in cobalt radioisotopes with time and the corresponding change in ⁶¹Cu purity.

TABLE 6
Cobalt isotopes: enriched ⁶⁰Ni/Nb target coin.

| | ⁶¹Cu fraction | | Separated Co-waste | |
|---|---|---|---|---|
| Radionuclide | [Bq] | Separated Ni [Bq] | I + II [Bq] | Half-life [days] |
| ⁵⁶Co | 365 | 8 | 7583 | 77 |
| ⁵⁷Co | 0 | 0 | 1793 | 272 |
| ⁵⁸Co | 242909 | 5463 | 4940424 | 71 |
| ⁶⁰Co | 0.5 | 0 | 11 | 1925 |

### 5.1.8. Activity and Chemical Purity

Based on measured activities (MBq) at different beam currents (μA) and timescales (5-60 minutes), the measured activity resulting from deuteron bombardment of ^{nat}Ni, ⁶⁰Ni and proton bombardment of ⁶¹Ni using the process described herein was found to be approximately ≥80% of the theoretical activity calculated using the TENDL-19 cross section database.

The activity of radiocobalt and other long-lived radionuclides was measured post-release (≥3 weeks after bombardment). The EOB activity of the long-lived impurities was then extrapolated.

In Table 7, the extrapolated radiocobalt activity content and ⁶¹Cu purity of [⁶¹Cu]CuCl₂ solution produced by ^{nat}Ni as target metal for a 50 μA, 3 h deuteron irradiation after FASTlab purification were presented.

TABLE 7
Natural Ni/Nb Target Coin - Extrapolation of ⁶¹Cu activity
and purity in produced [⁶¹Cu]CuCl₂ solution.

| | Co species | | | | | |
|---|---|---|---|---|---|---|
| Hours | activity in | ⁶¹Cu | ⁶⁴Cu | | % Purity PET | |
| post | Cu fraction | activity | activity | % Purity | nuclides | % non-Cu |
| EoB | [Bq] | [MBq] | [MBq] | ⁶¹Cu | ⁶¹Cu + ⁶⁴Cu | radionuclides |
| 0 | 213864 | 4622 | 70 | | 99.995% | 0.00456% |
| 1 | 213784 | 3756 | 66 | 98.261% | 99.994% | 0.00559% |
| 2 | 213704 | 3052 | 63 | 97.979% | 99.993% | 0.00686% |
| 3 | 213624 | 2479 | 59 | 97.652% | 99.992% | 0.00841% |
| 4 | 213544 | 2015 | 56 | 97.274% | 99.990% | 0.01031% |
| 5 | 213465 | 1637 | 53 | 96.837% | 99.987% | 0.01263% |
| 6 | 213385 | 1330 | 50 | 96.332% | 99.985% | 0.01545% |
| 7 | 213305 | 1081 | 48 | 95.750% | 99.981% | 0.01890% |
| 8 | 213225 | 878 | 45 | 95.081% | 99.977% | 0.02309% |
| 9 | 213145 | 714 | 43 | 94.312% | 99.972% | 0.02817% |
| 10 | 213066 | 580 | 41 | 93.432% | 99.966% | 0.03434% |

Less than 0.03% non-Cu radioisotopes (⁵⁶Co and ⁵⁸Co) will be left in the copper fraction, assuming a product expiry time, e.g., ≥3 weeks post EoB. This value was lower than the limit allowed for Ga-68 cyclotron-produced as found in the Pharmacopeia (*0.1% at expiry for non-Ga radioisotopes):

The ⁶⁴Cu originating from natNi irradiation (content ˜5% at expiry) will be the main impurity, reducing the radioisotopic purity of ⁶¹Cu product at longer irradiation times or shelf-life (illustrated as the grey curve in FIG. 3).

Table 8 and FIG. 4 show the extrapolated radiocobalt activity content and ⁶¹Cu purity of the produced [⁶¹Cu]CuCl₂ solution after FASTlab purification.

TABLE 8
⁶⁰Ni/Nb Target coin - Extrapolation of ⁶¹Cu activity
and purity in produced [⁶¹Cu]CuCl₂ solution.

| | Co species | | | | | |
|---|---|---|---|---|---|---|
| Hours | activity in | ⁶¹Cu | ⁶⁴Cu | | % Purity PET | |
| post | Cu fraction | activity | activity | % Purity | nuclides | % non-Cu |
| EoB | [Bq] | [MBq] | [MBq] | ⁶¹Cu | ⁶¹Cu + ⁶⁴Cu | radionuclides |
| 0 | 243275 | 17498 | 0.378 | | 99.999% | 0.00139% |
| 1 | 243176 | 14217 | 0.358 | 99.996% | 99.998% | 0.00171% |
| 2 | 242977 | 11552 | 0.339 | 99.995% | 99.998% | 0.00210% |
| 3 | 242680 | 9386 | 0.321 | 99.994% | 99.997% | 0.00259% |
| 4 | 242285 | 7627 | 0.304 | 99.993% | 99.997% | 0.00318% |
| 5 | 241792 | 6197 | 0.288 | 99.991% | 99.996% | 0.00390% |
| 6 | 241201 | 5035 | 0.272 | 99.990% | 99.995% | 0.00479% |
| 7 | 240514 | 4091 | 0.258 | 99.988% | 99.994% | 0.00588% |
| 8 | 239731 | 3324 | 0.244 | 99.985% | 99.993% | 0.00721% |
| 9 | 238853 | 2701 | 0.231 | 99.983% | 99.991% | 0.00884% |
| 10 | 237882 | 2195 | 0.219 | 99.979% | 99.989% | 0.01084% |

Less than 0.01% non-Cu radioisotopes (⁵⁶Co and ⁵⁸Co) were left in the Cu fraction, assuming a product expiry time of 8 h post EoB. This value was ten times lower than the allowed limit for ⁶⁸Ga cyclotron-produced as found in the Pharmacopeia (0.1% at expiry for non-Ga radioisotopes*).

Less than 0.02% ⁶⁴Cu was left in the copper fraction at an expiry time of 8 h post EoB, one hundred times lower than the specification required for ⁶⁸Ga (2% Ga radioisotopes were allowed for ⁶⁸Ga).

5.1.9. Purity of produced [⁶¹Cu]CuCl₂ from Ni/Nb target coins: Comparison with Commercially Available Radionuclides

In Table 9, a comparison of the regulatory specifications on the purity of commercially available radionuclides are given along with the characteristics of the high purity [⁶¹Cu]CuCl₂ produced from deuteron irradiation of ^{nat}Ni/Nb and enriched ⁶⁰Ni/Nb target coin (50 μA, 3 h) and after FASTlab purification described herein.

TABLE 9
Comparison between commercially available radionuclides and [⁶¹Cu]CuCl₂
solution produced from irradiation of ^{nat}Ni/Nb coins and enriched ⁶⁰Ni/Nb coins.

| | | % Max | | | | |
|---|---|---|---|---|---|---|
| | | radioisotopes | % Max other | | | |
| | % Purity at | of same | radioisotopes | | | % Max other |
| | EoB + 2 | element at | at EoB + | Dominant | % Purity | radioisotopes |
| Radionuclide | hours | EoB + 2 hours | 2 hours | impurities | at expiry | at expiry |
| ¹¹¹In | 99.93% | 0.075% | | ⁶⁵Zn, ^{114m}In | 99.85% | 0.15% |
| ¹⁸F | | | | ⁵⁶Co | 99.90% | 0.10% |
| ¹⁸F | | | | ⁵⁶Co | 99.99% | 0.01% |
| ⁶⁸Ga | 98% | 2% | 0.10% | | | |
| cyclotron | | | | | | |
| ⁶⁸Ga | 99.90% | | 0.001% | ⁶⁸Ge | | |
| generator | | | | | | |
| ¹⁷⁷Lu | 99.90% | 0.05% | | | | |
| ⁶¹Cu from | 97.27% | 3.16% | 0.013% | ⁵⁶Co, ⁵⁸Co | 95.08% | 5% |
| ^{nat}Ni | (EoB + 4 h) | | | | (EoB + 8 h) | |
| ⁶¹Cu from | 99.99% | 0.009% | 0.004% | ⁵⁶Co, ⁵⁸Co | 99.98% | 0.02% |
| ⁶⁰Ni | (EoB + 4 h) | | | | (EoB + 8 h) | |

As the first notable comparison, cyclotron production of ⁶⁸Ga from proton irradiation also produces long lived radionuclides, (see, e.g., Applied Radiation and Isotopes, 65 (10), 1101-1107, IAEA-TECDOC-1863 Gallium-68 Cyclotron Production) notably ⁶⁵Zn (half-life=244 days) from the ⁶⁶Zn(p,pn) ⁶⁵Zn decay. With a roughly 0.365% of ⁶⁶Zn in an enriched 68Zn starting target metal, about 770 Bq of ⁶⁵Zn will be produced from a 50 μA, 3 h beam with an energy of 13 MeV in a thick target (TENDL-2019 based calculations). Using natural Zn with 27.7% abundancy in ⁶⁶Zn, 58 kBq of ⁶⁵Zn will be produced in one run of 50 μA for 3 h beam.

Similar with [⁶¹Cu]CuCl₂ production, cyclotron production of [⁶⁴Cu]CuCl₂ from proton irradiation also produces long-lived cobalt radionuclides, namely, ⁵⁵Co, ⁵⁷Co, ⁵⁸Co, and ⁶⁰Co. (See, e.g., Nuclear Medicine & Biology, Vol. 24, pp. 35-43, 1997; Applied Radiation and Isotopes 68 (2010) 5-13). By operating with a degraded beam of below 13 MeV, ⁶⁰Co (from ⁶⁴Ni(p,na)⁶⁰Co) was reduced to 1 Bq per run of 50 μA, 3 h. With beam energies below 13 MeV, ⁵⁵Co, formed from the ⁵⁸Ni(p,a)⁵⁵Co reaction, will remain the main impurity (half-life=17.53 hours). The 170 Bq of the long-lived ⁵⁷Co was formed in about 170 Bq in these conditions mostly from ⁶⁰Ni(p,a) ⁵⁷Co.

Note: These estimates were computed from thick target yields using TENDL-2019 cross section data and isotopic abundancy of enriched ⁶⁴Ni as follows: 0.00376% ⁵⁸Ni, 0.00298% ⁶⁰Ni, 0.0058% ⁶¹Ni, 0.135% ⁶²Ni, 99.858% ⁶⁴Ni.

### 5.1.10. Enriched ⁶¹Ni as Target Metal on Nb Backed Coins

⁶¹Cu was produced through the proton bombardment of ⁶¹Ni electroplated Nb backed coin via cyclotron equipped with a solid target system irradiating a highly pure Niobium coin plated with highly pure ⁶¹Ni (purity 99.42%). The proton beam currents used were up to 100 μA, and beam energy of 13 MeV. An aluminum beam degrader was used.

The solid target irradiated material was dissolved in a total volume of 7 mL of 6M HCl with the addition of 30% H₂O₂ in a heated dissolution chamber. The ⁶¹Cu was purified from metal and radiometal impurities via a GE Healthcare FASTlab 2 module through a tributyl phosphate resin cartridge and a tertiary-amine-based weak ionic exchange resin containing long-chained alcohols. The product was finally eluted in an ISO class 5 environment in 3 mL 0.05 M HCl through a sterile filter Millex 4 mm Durapore PVDF 0.22 μm into a sterile evacuated vial. The vial was handled with care using the appropriate shielding and can be stored at room temperature until use using appropriate shielding for transport and handling. The properties of the [⁶¹Cu]CuCl₂ solution were determined and are displayed below in Table 10.

TABLE 10
[⁶¹Cu]CuCl₂ produced from ⁶¹Ni.

| Parameter | Test Method | Specification | |
|---|---|---|---|
| Appearance | Visual inspection | Clear, colorless solution, free from | |
| | | particulate matter | |
| Volume | Weight measurement | 3 ± 0.3 | mL |
| Activity concentration | Dose calibrator | 0.20-2 | GBq/mL |
| (EoS) | | | |
| pH value | pH paper strips | 1-1.6 | |
| Radiochemical purity | Radio-TLC | ≥99% (as [⁶¹Cu]CuCl₂) | |
| Radionuclidic identity | γ-Spectrometry (in lab at EoB + 90 | γ-photons with energy peaks at: | |
| | minutes) | 283 keV ± 20 keV | |
| | | 511 ± 20 keV (eventually sum | |
| | | peak at 1022 keV ± 20 keV) | |
| | | 656 keV ± 20 keV | |
| | Half-life via dose calibrator | 200 ± 20 | min |
| Radionuclidic purity | γ-Spectrometry (sent out, | ≥99.9% | |
| | evaluated >3 weeks, values extrapolated | | |
| | to in lab at EoB + 90 minutes) | | |
| Bacterial endotoxin | LAL test (Endosafe) | ≤17.5 | EU/mL |
| content | | | |
| Chemical purity | ICP-MS (sent out, evaluated >3 weeks, | Sum of impurities ≤15 μg/GBq | |
| | values relevant to in lab at EoB + 90 | Cu ≤ 0.5 | μg/GBq |
| | minutes as these do not change with | Al ≤ 2 | μg/GBq |
| | time) | Co ≤ 1 | μg/GBq |
| | | Fe ≤ 3 | μg/GBq |
| | | Pb ≤ 1 | μg/GBq |
| | | Ni ≤ 2 | μg/GBq |
| | | Zn ≤ 1 | μg/GBq |

*post-release (≥3 weeks)
^{#}measured periodically

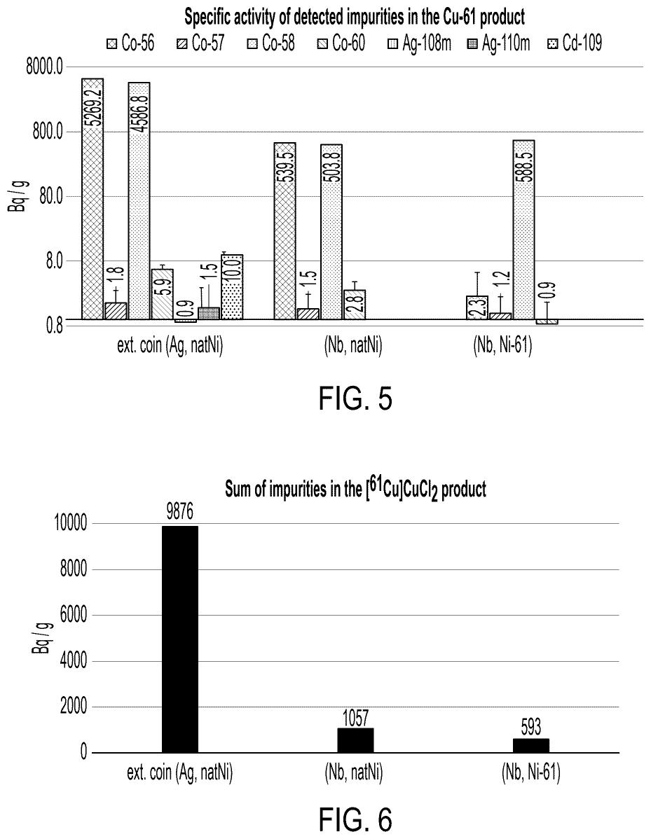

As shown in Table 11 and FIG. 5, commercially available [⁶¹Cu]CuCl₂ contains radionuclidic impurities, particularly high levels of ⁵⁶Co and ⁵⁸Co, in addition to ^{110m}Ag and ¹⁰⁹Cd. Elimination of Ag and Cd isotopes from the Cu-61 product was achieved by replacing silver with niobium as backing material. There was a nine-fold reduction of ⁵⁶Co isotopes for ^{nat}Ni and ≥2000× reduction for Ni-61 (less shielding of radioactive waste is required). 50% reduction of long-lived cobalt isotopes (earlier final disposal of the produced waste) was also observed. It was clear from the data below, that the radionuclidic purity of [⁶¹Cu]CuCl₂ produced by the methods described above to be superior to previously known methods and products. The high levels of long-lived Co, Ag, and Cd radionuclides pose a radiation burden for the patient and a radioactive waste issue for consumables that have come in contact with the [⁶¹Cu]CuCl₂ product during radiopharmaceutical manufacturing and radiolabeling.

TABLE 11
Detailed radionuclidic impurities present in commercially
available ⁶¹Cu compared to high-purity [⁶¹Cu]Cl₂
of the present disclosure, expressed in Bq/g.

| | | | Coin | | |
|---|---|---|---|---|---|
| | | | Ext. Coins | Present Coins | |
| | | | nat-Ni on Ag | nat-Ni on Nb | ⁶¹Ni on Nb |
| Elements | T_{1/2} | | Bq/g | | |
| Co-56 | 77.23 | days | 5269.2 | 539.5 | 2.3 |
| Co-57 | 271.74 | days | 1.8 | 1.5 | 1.2 |
| Co-58 | 70.86 | days | 4586.8 | 503.8 | 588.5 |
| Co-60 | 5.27 | years | 5.9 | 2.8 | 0.9 |
| Ag-108 m | 439 | years | 0.9 | N/D | N/D |
| Ag-110 m | 249.86 | days | 1.5 | N/D | N/D |
| Cd-109 | 462.6 | days. | 10 | N/D | N/D |

The total radionuclidic impurity profile was summed (Table 12 and FIG. 6). There was an 83% decrease in radionuclidic impurities. When present in the [⁶¹Cu]CuCl₂ product, these impurities can cause a radiation burden for the patient, waste issues, and degrade the quality of, e.g., a radiotracer or radiopharmaceutical. They can also interfere with the chelation process by competing with ⁶¹Cu, which affects the accurate radiolabeling of the tracer. An 89.3% reduction of impurities was observed upon changing the backing material from silver to the niobium backing provided herein and using the Ni plating methods described herein. An Additional reduction of 46% was observed when using Ni-61 as starting material.

TABLE 12
Radionuclidic impurities in the produced [⁶¹Cu]CuCl₂.

| Coin | |
|---|---|
| Ext. Coins | Present Coins |

| | natNi on | nat-Ni on | |
|---|---|---|---|
| | Ag. | Nb | ⁶¹Ni on Nb |
| Bq/g | 9876 ± 1.5 | 1057 ± 1.8 | 593 ± 0.2 |

Consequent to the purity of the ⁶¹Cu at EoB and End of Synthesis (EOS, EoB+2), long-lived radionuclidic impurities decay slower and, thus, increase in concentration in relation to ⁶¹Cu at longer timescales. Thus, the impurity profile may vary greatly based on the isotopic enrichment of the raw material, purity, method, and process of producing a coin, which influences the type and amount of radionuclidic impurities in the finished [⁶¹Cu]CuCl₂ product.

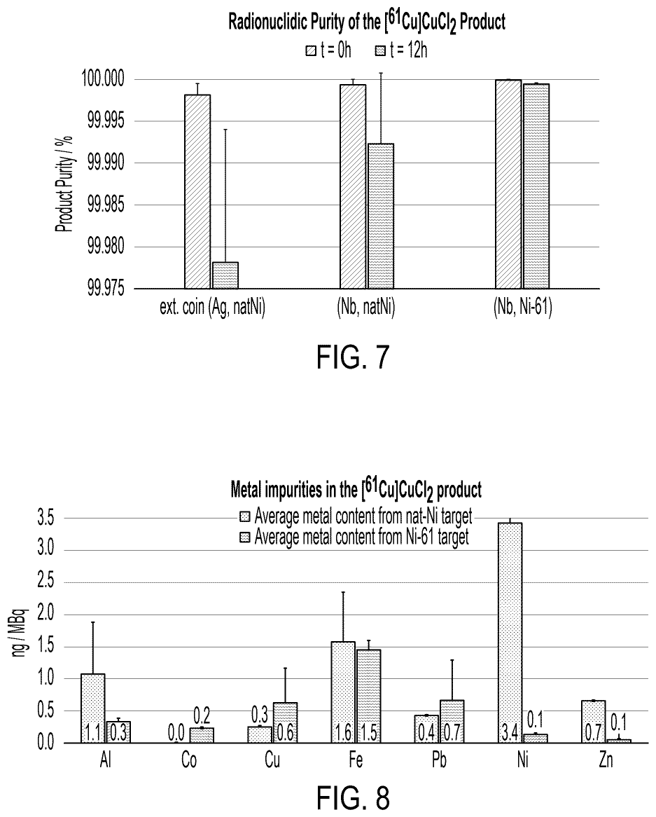

FIG. 7. contrasts the radionuclidic purity of [⁶¹Cu]CuCl₂ solution produced with commercially available natNi target metal on a Ag backing compared to the radionuclidic purity of [⁶¹Cu]CuCl₂ solution produced by irradiation of Ni target metal electroplated according to the present disclosure on high purity Nb backing when assessed by gamma spectrometry in Bq/g (summed radionuclidic impurities) at t=Oh and at t=12 h. The presented data highlight the superior quality of the [⁶¹Cu]CuCl₂ solution when produced by irradiation of Ni target coatings electroplated according to the present disclosure on high purity Nb backing, where the purity after 12 hours is still well above the purity limits set by pharmacopeia for similar radionuclides for medical use. “End of Production” or “EoP” refers to the end of the preparation of [⁶¹Cu]CuCl₂.

TABLE 13
Radionuclidic purity of commercially available ⁶¹Cu
compared to high-purity [⁶¹Cu]CuCl₂ of the present
disclosure as measured at EoP and EoP + 12 hours.

Coin

Present Coins

| | Ext. Coins | nat-Ni on | |
|---|---|---|---|
| | nat-Ni on Ag | Nb | ⁶¹Ni on Nb |
| Purity % t = 0 h | 99.998 | 99.999 | 99.9999 |
| Purity % t = 12 h | 99.978 | 99.993 | 99.999 |

### 5.1.11. Conclusion

The experimental activities of ⁶¹Cu produced after deuteron irradiation were about 80% of the theoretical yield as calculated from TENDL-2019 cross section data.

The main long-lived nuclides in the radioactive waste fraction from cyclotron production of ⁶¹Cu were radiocobalt species of ⁵⁶Co, ⁵⁷Co, ⁵⁸Co, and ⁶⁰Co. It was calculated that, after four years, ⁵⁶Co, ⁵⁷Co, and ⁵⁸Co will have decayed below regulatory clearance limits, LL*, leaving only ⁶⁰Co. * Clearance limits (LL) means the value corresponding to the activity concentration level of a material below which handling of this material is no longer subject to mandatory licensing or supervision.

The yield and purity of [⁶¹Cu]CuCl₂ prepared with niobium coins was improved by plating the niobium coins with 99% enriched ⁶⁰Ni or ⁶¹Ni. The purity of [⁶¹Cu]CuCl₂ product was higher as ⁶⁴Cu was be formed as a radioisotopic impurity. Additionally, the ⁵⁶Co and ⁶⁰Co contents were reduced by a factor of 100. 57Co amounts increased (but were low activity) and ⁵⁸Co amounts doubled (but decay below LL before ⁵⁶Co/⁵⁸Co).

5.1.12. Batch Control of [⁶¹Cu]CuCl₂

Three representative batches of [⁶¹Cu]CuCl₂ solution were manufactured as described above (by irradiating a solid target consisting of highly pure ⁶¹Ni (purity ≥99.42%) plated on a niobium coin backing with a proton beam) and tested. The results of these analyses are presented in Table 14. “End of Production” or “EoP” refers to the end of the preparation of [⁶¹Cu]CuCl₂.

TABLE 14
Analyses of three batches of [⁶¹Cu]CuCl₂.

| | Specification | | Results | | | | | |
|---|---|---|---|---|---|---|---|---|
| Batch | — | | 1 | | 2 | | 3 | |
| Volume | 3 ± 0.3 | mL | 3.04 | mL | 3.07 | mL | 3.03 | mL |
| Activity | 0.50-2.00 | GBq/mL | 0.96 | GBq/mL | 1.18 | GBq/mL | 1.21 | GBq/mL |
| concentration | | | | | | | | |
| (EoP) | | | | | | | | |
| Apparent | ≥30 | MBq/nmol | passed | | passed | | passed | |
| molar activity^{#} | | | | | | | | |
| pH value | 1-1.6 | | 1.3 | | 1.2 | | 1.3 | |
| Radiochemical | ≥99% (as | | 99.88% | | 99.47% | | 99.79% | |
| purity | [⁶¹Cu]CuCl₂) | | | | | | | |
| Radionuclidic | γ-photons with | | 288 | keV | 286 | keV | 288 | keV |
| identity | energy peaks at: | | 512 | keV | 512 | keV | 511 | keV |
| | 283 keV ± 20 keV | | 658 | keV | 656 | keV | 660 | keV |
| | 511 ± 20 keV | | | | | | | |
| | (eventually sum | | | | | | | |
| | peak at 1022 keV ± | | | | | | | |
| | 20 keV) | | | | | | | |
| | 656 keV ± 20 keV | | | | | | | |
| Half-life | 200 ± 20 | min | 193.8 | min | 192 | min | 195.6 | min |
| Radionuclidic | ≥99.9% | | 99.986% | | 99.994% | | 99.988% | |
| purity* | | | | | | | | |
| Radionuclidic | Sum of | | Sum of impurities = | | Sum of impurities = | | Sum of impurities = | |
| Impurities | impurities ≤ 0.1% | | 1.4 × 10⁻²% | | 0.6 × 10⁻²% | | 1.2 × 10⁻²% | |
| [^{57, 58}Co]Co | ⁵⁷Co ≤ 0.1% | | ⁵⁷Co < 4.0 × 10⁻⁵% | | ⁵⁷Co < 2.9 × 10⁻⁵% | | ⁵⁷Co < 3.8 × 10⁻⁵% | |
| | ⁵⁸Co ≤ 0.1% | | (below LOQ) | | (below LOQ) | | (below LOQ) | |
| | | | ⁵⁸Co = 1.4 × 10⁻²% | | ⁵⁸Co = 0.6 × 10⁻²% | | ⁵⁸Co = 1.2 × 10⁻²% | |
| Bacterial | ≤17.5 | EU/mL | ≤3.75 | EU/mL | ≤3.75 | EU/mL | ≤3.75 | EU/mL |

| | | | | |
|---|---|---|---|---|
| endotoxin | | | | |
| content | | | | |
| Chemical | Co ≤ 5 μg/GBq | Co < 0.05 μg/GBq | Co < 0.04 μg/GBq | Co < 0.04 μg/GBq |
| purity* | Ni ≤ 20 μg/GBq | (below LOQ) | (below LOQ) | (below LOQ) |
| | | Ni = 0.40 μg/GBq | Ni = 0.31 μg/GBq | Ni = 0.29 μg/GBq |

*post-release.
^{#}measured periodically

### 5.1.13. Radionuclidic Purity

Radionuclidic purity is important in radiopharmacy since any radionuclidic impurities increase the radiation dose received by the patient and may also degrade the quality of any imaging procedure performed. For example, if significant levels of other radionuclides are present then biological distribution may be altered. Radionuclide samples contain some contaminants arising the production process or the decay of the primary radioisotope. Radionuclide impurities can occur as a result of the manufacturing process, for example, for nuclides produced by cyclotron there can be contaminants due to impurities in the target or by the energy of the reaction. In order to control the effects of these contaminants on the radiation dose received by the patient, limits are set on the maximum levels of contamination allowed. These limits are defined by governmental agencies, e.g., in pharmacopoeia monographs, and vary depending upon the radionuclide concerned and the physical decay characteristics of the likely contaminants. Measurement of radionuclidic purity may be performed high resolution using gamma-ray spectroscopy on samples well after bombardment. The activity of the long lived isotopes is then extrapolated back to EoB, EoP, EOS, or even at expiration. High activity emitted from long lived radionuclidic impurities greatly increases the cost and complexity of managing the disposal of all consumables that come into contact with the nuclide composition.

Through the deuteron irradiation of natural nickel and ⁶⁰Ni, and proton irradiation of ⁶¹Ni, long-lived isotopes of cobalt are produced: ⁵⁶Co, ⁵⁷Co, ⁵⁸Co and ⁶⁰Co. Other long-lived radionuclides such as ^{110m}Ag, ^{108m}Ag and ¹⁰⁹Cd are produced through the irradiation of commonly used silver backing material, which are dissolved along with starting material during the purification process. Due to their long half-lives, the proportion of these radionuclides increases with time compared to the ⁶¹Cu, decreasing the radionuclidic purity of the product, especially at later time points when using ^{nat}Ni as a starting material. Though most cobalt isotopes can be separated in the purification process, the ^{110m}Ag, ^{108m}Ag and ¹⁰⁹Cd end up in the ⁶¹Cu fraction and nickel solution that is further used in recycling of irradiated target coating. The long-lived radionuclides become problematic when considering the radiation burden to the patient and the accumulation of radioactive waste. Third-party coin manufacturers did not publish the contamination from the non-niobium coin backings (e.g., silver). As provided by the present disclosure, the method of making and using coins comprising niobium represents an advantage, e.g., in view of the radionuclidic and chemical purity of samples produced following subatomic particle bombardment, isolation, and purification. A detailed comparison of the known ⁶¹Cu products (prepared via Ag backings and prior art methods of plating the target) to ⁶¹Cu as provided by the present disclosure is provided below.

With these factors in mind, a niobium backing material was chosen due to its inert nature to acids at room temperature and at elevated temperatures. This characteristic allows the niobium backing material to resist the acid medium used during the dissolution and purification process. By doing so, higher radionuclidic and chemical purity can be achieved in the radiometal aqueous solution, eventually resulting in higher purity for the radiopharmaceutical prepared from the desired ⁶¹Cu isotope. Although plating methods of niobium exist, the element has not yet been used for radionuclide production due to the poor adhesion of the plated Ni material (as discussed above). The Ni (or 68Zn for the production of ⁶⁸Ga) requires sufficient adhesion for the coin to survive thermal loads (1200 W) during irradiation and pneumatic shuttle acceleration at 5 bar to 7 bar of pressure and abrupt stop at the head. On the other hand, however, the plated Ni (or Zn) must dissolve sufficiently during the dissolution and purification process. Attempts were made to plasma-coat niobium backings for plating nickel (Ni). However, this process resulted in losses and incomplete dissolution of Ni from the niobium backing. The thermal processes involved in plasma coating altered the grain structure of the niobium backing material, leading to a strong bond between the plated nickel and niobium. This strong bond made it difficult for the nickel to fully dissolve, causing losses. The plasma coating process itself resulted in very high losses in target coating, rendering the process not viable for use, especially with very expensive highly enriched target metals. The main reference to this summary is the IAEA documentation regarding cyclotron radionuclide production, IAEA RADIOISOTOPES AND RADIOPHARMACEUTICALS, REPORTS, No. 1. (INTERNATIONAL ATOMIC ENERGY AGENCY VIENNA, 2016) Additionally, a monetary evaluation regarding the procurement costs of niobium utilized as a backing material displays a 40% lower cost in comparison to commonly used backing materials such as gold, silver, and platinum where costs range from €80 to €120 per backing material (single coin).

Parallel to this, elements pertaining to the radiochemical purity of the labelling process are controlled by manufacturing the plating solution under controlled conditions described herein. By procuring the plating solution from a raw base material of, e.g., nickel, the possibility of contamination is now independent from outside sources and suppliers. Such material and equipment used in these cases are inert glass beakers and falcon tubes (ensured to not contain any undesirable substances), TraceSelect pure water, pure reagents (trace-metal grade), inert coin adapter and electrolytic cell (on the electroplating unit), etc. Through this, the contaminants of trace metals can be minimized reduced or avoided all together. This difference between 99.9% purity and 99.99% purity plays a role in the resulting chemical purity of a radionuclide and therefore in the radiochemical purity of, e.g., a radiotracer or radiopharmaceutical prepared from the radionuclide, where the presence of cold Cu, Zn, Fe, Sn, Ti, or Al or any salt thereof are an issue as they will compete for binding to the chelator in the tracer along with the desired radionuclide (⁶¹Cu).

Robustness of plating was tested through a drop and scratch test. This assessment ensures that the electrodeposited substrate on the backing will survive mechanical impacts of the shuttling system and established an increased probability of survivability under the cyclotron beam.

In certain embodiments, coins were irradiated with 8.4 MeV deuterons for an average duration of 120 mins at a range of 40 μA to 45 μA or with 13.2 MeV deuterons at 40 μA to 45 μA using an ARTMS or GE shuttling system on a GE PET Trace cyclotron.

In certain embodiments, the coins were irradiated with 8.4 MeV deuterons for an average duration of 120 mins at a range of 40 μA to 45 μA or with 10 μA to 100 μA 13 MeV protons using an ARTMS or GE shuttling system on a GE PET Trace cyclotron.

Dissolution of Ni from the niobium backing was accomplished via the utilization of a dissolution system in 10 M HCl. The subsequent ⁶¹Cu was then purified with two subsequent ion exchange resins in a FASTlab synthesis unit. The processing time for these purifications can reach up to 60 minutes.

The resulting [⁶¹Cu]CuCl₂ solution of the plated material has an average activity of 1.7-4.5 GBq. This activity was measured using a dose calibrator and its radionuclidic purity by a calibrated gamma spectrometer e.g., at PSI in Switzerland.

Gamma spectrometry measurements were performed to identify any radionuclidic impurities, particularly long-lived radionuclides. These results indicate an 89.3% and 94% reduction in impurities for ^{nat}Ni and ⁶¹Ni on niobium backing materials with respect to silver backing materials when utilizing the methods disclosed herein. ICP-MS measurements are performed on the product of cold dissolutions by Labor Veritas in Switzerland to monitor elemental impurities present in the product according to ICH-Q3D. All detected impurities are within regulated ICH-Q3D concentrations (see ICH-Q3D Guidelines, pg 25).

The plating of highly enriched ⁶¹Ni was also enabled with the same plating parameters as described above, for a higher yield and industrial production using proton irradiation (typically at 10 μA to 100 μA. 13 MeV protons for 20 minutes to 2 hours and up to one half-life of ⁶¹Cu).

Following automated transportation of the irradiated coin from the cyclotron to the hot cell docking station, the capsule was transferred to a QIS dissolution unit with tongs. The transmuted target metal was dissolved from the niobium backing material using 1:1 7M HCl: 30% H₂O₂ (ultratrace analysis, Merck) (4 mL). The acid-peroxide mixture is circulated, immersing the coin and target metal surface to dissolve all irradiated elements at 2 mL/min for about 23 minutes at about 60° C. When the target metal was fully dissolved, acidic solution containing the dissolved metal was withdrawn and the QIS system was flushed with 10M HCl (3 mL). The combined acidic solutions were then fed forward to the FASTlab purification unit.

For this reason, gamma spectrometry analyses were carried out on decayed samples (at least 10 half-lives of ⁶¹Cu, corresponding at the earliest to 1.4 d after EoP). The analysis was performed using a high-purity germanium (HPGe) detector GEM30-70 from Ortec.

### 5.1.14. Activity Concentration

The activity of the [⁶¹Cu]CuCl₂ solution was quantified using a dose calibrator following Ph. Eur. 2.2.66 guidelines. The test was carried out at the end of the radionuclide production (EoP) using a certified dose calibrator, selecting the ⁶¹Cu measuring channel. The weight of the [⁶¹Cu]CuCl₂ solution was measured using an analytical scale. Assuming a density of the aqueous solution of 1.0 g/mL, the weight is converted into a volume. The activity concentration, expressed in GBq/mL, is then calculated by dividing the activity by the volume.

### 5.1.15. Apparent Molar Activity

The suitability of the [⁶¹Cu]CuCl₂ solution for radiolabeling was ensured by determining the apparent molar activity. This test assessed the impact of competing trace metals on ⁶¹Cu chelation by quantifying the minimum amount of a given chelator required for efficient radiolabeling. The test can be performed with various chelators that can complex copper (e.g., NOTA, DOTA, NODAGA). Among them, NODAGA has been assessed for ⁶¹Cu molar activity. The test measures the percentage of complexation by radio-TLC after reacting a fixed amount of [⁶¹Cu]CuCl₂ solution with different amounts of chelator (titration). The results are then plotted (x axis (logarithmic): nmol of chelator; y axis: % of complexation), and a sigmoidal curve is obtained. The EC₅₀ point is extrapolated, corresponding to the amount of chelator needed to achieve the 50% of complexation. The apparent molar activity is calculated by dividing the ⁶¹Cu activity used extrapolated at the end of production (EoP) by 2 times the EC₅₀ value.

This test was not performed as a routine analysis, but when changes occur in 1) the grade of a chemical employed for the manufacturing of the [⁶¹Cu]CuCl₂ solution, 2) the quality of a consumable employed for the manufacturing of the [⁶¹Cu]CuCl₂ solution, 3) the target coin manufacturing process, and 4) if poor radiolabeling yields were observed. The AMA test is specific to a chelator and labelling conditions (e.g., buffer concentration and pH) and has thus to be repeated when these parameters are modified.

5.1.16. pH

The pH value was determined by colorimetric evaluation using pH paper strips with a narrow range (pH interval 0-2.5) exposed to a 10 μL of the [⁶¹Cu]CuCl₂ solution. The color of the paper strips was compared to the reference color scale displayed on the strips container.

### 5.1.17. Radiochemical Purity (Radio-TLC)

The radiochemical purity of a [⁶¹Cu]CuCl₂ solution prepared as described herein was determined by radio-TLC following Ph. Eur. 2.2.66 guidelines. The test determined the percentage of ⁶¹Cu present in the desired ionic form, namely as free Cu²⁺. The retention factor (R_{f}) was determined as the distance from the origin to the peak divided by the distance from the origin to the solvent front. Ionic [⁶¹Cu]Cu²⁺ migrated to the solvent front (R_{f}=0.8-1.0), while colloidal [⁶¹Cu]Cu(OH) 2 remained at the point of application (R_{f}=0.0-0.2). The specification required that ≥99% of the whole radioactivity detected was present in the ionic form [⁶¹Cu]Cu²⁺.

For the test, 2 L of the radioactive solution was applied at 1 cm away from the lower end of an iTLC paper plate (8×1 cm), consisting of glass microfiber chromatography paper impregnated with silica gel. After drying, the iTLC plate was placed in a glass chamber where it was run in a 0.1 M citrate buffer solution (pH 5). When the solvent front had reached a distance of approximately 1 cm from the top of the iTLC plate, the plate was removed from the chamber and scanned using a PET miniGita Star from Elysia-Raytest, equipped with a β-sensitive detector and controlled by the software Gina Star from Elysia-Raytest.

### 5.1.18. Gamma Spectrometry (Radionuclidic Identity)

The radionuclidic identity of a [⁶¹Cu]CuCl₂ solution prepared as described herein was confirmed by gamma spectrometry following Ph. Eur. 2.2.66 guidelines. The presence of the main γ-photons with energy peaks characteristic of ⁶¹Cu (listed in Table 15) was assessed. The table also lists the energy peak of the γ-photons belonging to ⁵⁸Co, which represents the main impurity detected in the test. The test was carried out at EoP using a Mucha Star multichannel analyser from Elysia-Raytest, equipped with a NaI detector, and controlled by the Gina Star from Elysia-Raytest software.

TABLE 15
Energy peaks characteristic of ⁶¹Cu and of its main impurity ⁵⁸Co.

| Peak | Energy (keV) |
|---|---|
| Cu-61 | 67 |
| Cu-61 | 283 |
| Beta peak | 511 |
| Cu-61 | 656 |
| Co-58 | 811 |
| Sum peak | 1022 |
| Cu-61 | 1185 |

### 5.1.19. Half-Life (Radionuclidic Identity)

The radionuclidic identity of a [⁶¹Cu]CuCl₂ solution prepared as described herein was further confirmed by half-life determination following Ph. Eur. 2.2.66 guidelines. The test was carried out at the end of the radionuclide production (EoP) using a certified dose calibrator from Comecer (model VDC-505), selecting the ⁶¹Cu measuring channel. The specification required the measured half-life value to be within a predefined range of the accepted half-life value (20%).

### 5.1.20. LAL Test (Bacterial Endotoxin Content)

The bacterial endotoxins were determined in a [⁶¹Cu]CuCl₂ solution prepared as described herein by limulus amoebocyte lysate (LAL) test following Ph. Eur. 2.6.14 guidelines. This system applies LAL kinetic chromogenic methodology that measures color intensity directly proportional to the endotoxin concentration in the sample. Each cartridge contained predefined amounts of LAL reagent, chromogenic substrate, and control standard endotoxin (CSE). The LAL reagent was mixed automatically by the device with the sample or the positive product control. The mixtures were incubated and then combined with the chromogenic substrate. For quantification, the optical density of the substrate was measured and analyzed against an internally archived standard curve. The analysis was performed in duplicate for the sample as well as for the positive product control. The system contained an internal printer to generate a report.

### 5.1.21. Bioburden

The bioburden of the aqueous [⁶¹Cu]CuCl₂ manufacturing process described herein was evaluated following Ph. Eur. 2.6.12 guidelines. This test enables the detection and quantification of the viable microorganisms present in the system prior to terminal sterilization and represents a good reference point for evaluating the degree of safety of the process. The manufacturing process was performed in completeness but used non-irradiated target coins for radiation protection. The collected non-radioactive solution was analyzed by the Membrane-Filtration Method. Half of the sample was passed through a membrane filter with a pore size of 0.45 μm. The filter was placed onto Soybean-Casein Digest Agar and incubated to determine the total aerobic microbial count (TAMC). The other half of the sample was passed through a membrane filter with a pore size of 0.45 μm. The filter was then placed onto Sabouraud Dextrose Agar and incubated to determine the total yeast and mold count (TYMC). The bioburden was expressed in colony-forming units (CFU).

### 5.1.22. Control of Starting Material

Reagents and starting materials used to manufacture the radionuclide ⁶¹Cu are provided in Table 16. The reagents employed in production were of TraceSelect grade to minimize the trace metal impurities. These impurities could impact radiolabeling, which may result in a poor complexation of ⁶¹Cu with the chelator.

TABLE 16
Materials used to purify and formulate [⁶¹Cu]CuCl₂.

| Material | Quality |
|---|---|
| ⁶¹Ni | 99.42% enrichment |
| Niobium coin backing | 99.9% purity |
| ⁶¹Ni Coins (plated ⁶¹Ni | According to internal specifications |
| on Niobium) | |
| Hydrochloric acid | 30%, Ultrapure |
| Hydrogen peroxide | 30%, Suprapure |
| Sodium chloride | 99.99%, Suprapure |
| Water | Ultrapure |
| FASTlab cassette | Cassette's plastic components either |
| | sterile or tested for low bioburden |
| Solid Target Valve Box | Cassette's plastic components either |
| (STVB) cassette | sterile or tested for low bioburden |
| TBP and TK201 | Ion-exchange cartridge for purification |
| cartridges | |
| FASTlab water bag | Sterile WFI USP/Ph. Eur. |
| Vials | 20 mL sterile evacuated |
| Syringe needles | Sterile |
| Sterile filter Millex GV | 0.22 μm pore size, sterile |

At the end of the electroplating process, the target coin was examined using an optical microscope (20×, 50× and 250× magnification). All the steps of the coin manufacturing were performed using TraceSelect grade chemicals and metal-free consumables. The target coins were stored in a cool, dark environment in metal-free sealed containers.

### 5.1.23. Impurities

The impurities present in the irradiated starting material can undergo a nuclear reaction, leading to the formation of undesired radioactive species, as summarized in Table 17. Due to the high isotopic purity of the ⁶¹Ni(99.42% enrichment), these contaminations were limited.

TABLE 17
Composition of the ⁶¹Ni target coin
plating and main nuclear reactions.

| | |
|---|---|
| ⁶¹Ni composition | ⁵⁸Ni (0.004%), ⁶⁰Ni (0.3%), ⁶¹Ni (99.42%), |
| | ⁶²Ni (0.3%), ⁶⁴Ni (0.004%) |
| Desired nuclear reaction | ⁶¹Ni(p, n)⁶¹Cu |
| Contamination reactions | ⁶⁰Ni(p, α)⁵⁷Co (half-life: 271.74 d) |
| (selection of the main | ⁶¹Ni(p, α)⁵⁸Co (half-life: 70.86 d) |
| contributions) | ⁶¹Ni(p, n)⁶⁰Co (half-life: 1925 d) |

The presence of “cold” trace metal ions in the target coin material and reagents used during the dissolution and purification stages are to be limited. These trace metal ions can impact the subsequent radiolabeling processes by interfering with the complexation of ⁶¹Cu with the chelator. TraceSelect grade reagents and metal-free consumables are used with the aim of minimizing trace metal contamination.

The concentration of the trace metal ions in the [⁶¹Cu]CuCl₂ solution is determined by Inductively Coupled Plasma Mass Spectrometry (ICP-MS) (Table 18). ICP-AES can be used as alternative technique.

### 5.2. Example 2: [61Cu]Cu-NODAGA-LM3 Production

The GMP-grade precursor NODAGA-LM3 was synthesized (e.g., Fani M, et al. PET of somatostatin receptor-positive tumors using ⁶⁴Cu- and ⁶⁸Ga-somatostatin antagonists: the chelate makes the difference. J Nucl Med. 2011 July; 52 (7): 1110-8) and provided as a lyophilized powder in 2 mL vials containing 40 μg of product. The structure and nomenclature of the precursor NODAGA-LM3 are reported in Table 18.

TABLE 18
Structure and Nomenclature of NODAGA-LM3.

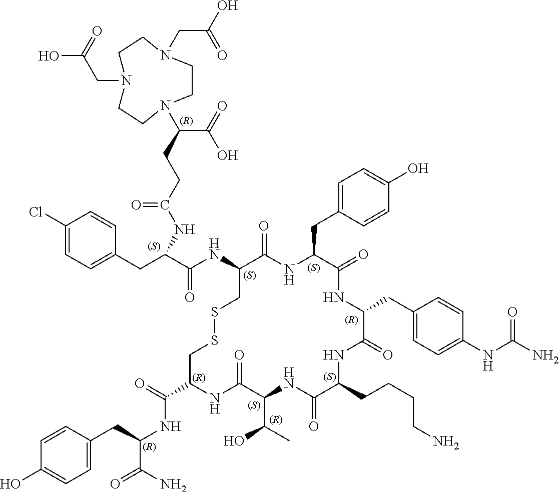

| | |
|---|---|
| Precursor Name | NODAGA-LM3 (TFA salt) |
| Chemical name | 2,2′-(7-((R)-4-(((S)-1-(((4R,7S,10S,13R,16S,19S)-4-(((R)-1-amino-3-(4- |
| (IUPAC) | hydroxyphenyl)-1-oxopropan-2yl)carbamoyl)-10-(4-aminobutyl)-16- |
| | (4-hydroxybenzyl)-7-((R)-1-hydroxyethyl)-6,9,12,15,18-pentaoxo-13- |
| | (4-ureidobenzyl)-1,2-dithia-5,8,11,14,17-pentaazacycloicosan-19- |
| | yl)amino)-3-(4-chlorophenyl)-1-oxopropan-2-yl)amino)-1-carboxy-4- |
| | oxobutyl)-1,4,7-triazonane-1,4-diyl)diacetic acid |
| Sequence | (R)-NODAGA-4-ClPhe-D-Cys-Tyr-D-Aph(Cbm)-Lys-Thr-Cys-D-Tyr- |
| | NH2 (cyclo 2-7) |

### Information on Heavy Metals

The presence of metal residues in NODAGA-LM3 is not expected, as no specific metal catalysts or metal-containing reagents are used during the production process. Potential contamination of starting materials by elemental impurities is not a concern, as the chelator is protected during the manufacturing procedure, and, therefore, chelation of metals is not possible. Multiple washing and filtration steps ensure that there are no elemental impurities present when the cleavage from the solid support occurs.

Although metal residues are not expected, to verify the absence of metal ions, one batch was tested for the presence of Cu, Fe, Ni, Pb, Zn, Pd, Cr and V. Measurements were performed using either inductively coupled plasma optical emission spectrometry (ICP-OES) for iron (Fe) or inductively coupled plasma mass spectrometry (ICP-MS) for all other metals.

The measurement of a bulk batch resulted in values far below the proposed limit of ≤0.01% (Table 19) for each metal impurity, which complies with the general monograph for chemical precursors for radiopharmaceutical preparations (Ph. Eur. 2902).

TABLE 19
Representative results of heavy metal analysis of NODAGA-LM3.

| Test parameter | Specification | Results |
|---|---|---|
| Heavy metals | ≤0.01% for each metal | Cu: <0.0030% |
| (IPC-MS; IPC- | impurity (Cu, Fe, Ni, Pb, | Fe: <0.0032% |
| OES) | Zn, Pd, Cr, V) | Ni: <0.0030% |
| | | Pb: <0.0015% |
| | | Zn: <0.0030% |
| | | Pd: <0.0015% |
| | | Cr: <0.0015% |
| | | V: <0.0015% |

The specifications of NODAGA-LM3 for bulk and bulk aliquots are presented in Table 20.

TABLE 20
Specifications for the drug substance NODAGA-LM3.

| | | |
|---|---|---|
| Test parameter | Specification | Analytical method |

Characters

| | | |
|---|---|---|
| Appearance | Bulk: White to off-white powder | Visual |
| | Bulk aliquots: White to off-white solid | |

Identification

| | | |
|---|---|---|
| MS-MS sequencing^{b} | Confirm sequence | MS-MS based on Ph. |
| | | Eur. 2.2.43¹ |
| Mass spectrometry | [MH]⁺_{m.i.} = 1520.6 ± 0.5 m/z | MALDI-TOF MS based |
| | | on Ph. Eur. 2.2.43¹ |
| Amino acid analysis^{b} | ClPhe to be reported | GC based on Ph. Eur. |
| | Cys 2.0 ± 0.4 μmol/μmol | 2.2.28¹ and Ph. Eur. |
| | Tyr 2.0 ± 0.4 μmol/μmol | 2.2.56¹ |
| | Aph to be reported | |
| | Lys 1.0 ± 0.2 μmol/μmol | |
| | Thr 1.0 ± 0.2 μmol/μmol | |

Assay

| | | |
|---|---|---|
| Peptide content* | To be reported | Determination based on |
| | | results of elemental |
| | | analysis of nitrogen (N) |
| | | based on Ph. Eur. 2.5.33¹ |
| Net peptide content | To be reported | Calculation: |
| (NPC) expressed as TFA | | 100% - % water - % TFA |
| free, anhydrous | | |
| substance* | | |
| HPLC assay (expressed | 80%-120% | RP-HPLC |
| as TFA free, anhydrous | complies to 32 μg-48 μg | based on Ph. Eur. 2.2.29¹ |
| substance) complies to | | |
| Filling quantity** | | |
| Peptide purity | ≥97.0% | RP-HPLC |
| | | based on Ph. Eur. 2.2.29¹ |

Impurities

| | | |
|---|---|---|
| Peptide related | No single impurities > 2.0% | RP-HPLC based on Ph. |
| substances/impurities | Reporting threshold ≥ 0.1% | Eur. 2.2.29¹ |
| Single impurities | Identification threshold > 2.0% | |
| Total related substances | Maximum 3.0% | Calculation sum of all |
| | | impurities ≥ 0.1% |
| Enantiomeric purity^{b} | ≤3% of L-enantiomer for D-Aph | GC-MS based on Ph. |
| | ≤3% of D-enantiomer for ClPhe, Thr and | Eur. 2.2.28¹ and Ph. Eur. |
| | Lys | 2.2.43¹ |
| | Note: determination of enantiomeric purity of | |
| | all other amino acids not feasible via GC-MS | |
| Residual organic | ≤0.5% CH₃CN | GC based on Ph. Eur. |
| solvents^{b,c} | ≤0.5% DMF^{a} | 2.2.28¹ |

Specific tests

| | | |
|---|---|---|
| TFA content^{b,c} | To be reported | GC or IC based on Ph. |
| | | Eur. 2.2.28¹ (GC) or Ph. |
| | | Eur. 2.2.29¹ (IC) |
| Water content | To be reported | GC based on Ph. Eur. |
| | | 2.2.28¹ |
| Bacterial endotoxins | Bulk: ≤1 EU/mg gross weight | LAL-test based on Ph. |
| | Bulk aliquots: ≤10 EU/container | Eur. 2.6.14¹ |
| Microbial | Bulk: | Microbial enumeration |
| contamination/bioburden | TAMC: ≤10² CFU/10 mg gross weight | test based on Ph. Eur. |
| | TYMC: ≤10¹ CFU/10 mg gross weight | 2.6.12¹ |
| | Bulk aliquots: | |
| | TAMC: ≤10² CFU/container TYMC: ≤10¹ | |
| | CFU/container | |

¹current version
*test valid for bulk, n.a. for bulk aliquots
**test valid for bulk aliquots, n.a. for bulk
^{a}to be determined for validation purpose
^{b}data for bulk aliquots deriving from bulk
^{c}data expected to be ≤ that at bulk release, report data ≤”result from bulk release

[⁶¹Cu]Cu-NODAGA-LM3 was obtained by radiolabeling the NODAGA-LM3 precursor with ⁶¹Cu in the form of [⁶¹Cu]CuCl₂. Lyophilized NODAGA-LM3 (80 μg, 56 nmol) was dissolved in 1.5 mL of 0.5 M sodium acetate and transferred into the vial containing the [⁶¹Cu]Cu(II) solution. The reaction mixture was then diluted with 2 mL of 0.9% sodium chloride (saline solution) with 2% v/v Vitamin C Streuli® and passed over a Waters Sep-Pak Light C18 SPE cartridge. The reaction vial and the RP cartridge were washed twice with 2-3 mL of the previously described sodium chloride solution. [⁶¹Cu]Cu-NODAGA-LM3 is finally eluted with 1.5 mL of an ethanol/water mixture (1:1 v/v) via a sterile filter into a product vessel, followed by 7.3 mL of the sodium chloride solution with Vitamin C Streuli®.

### 5.2.1. Automated Manufacturing Process

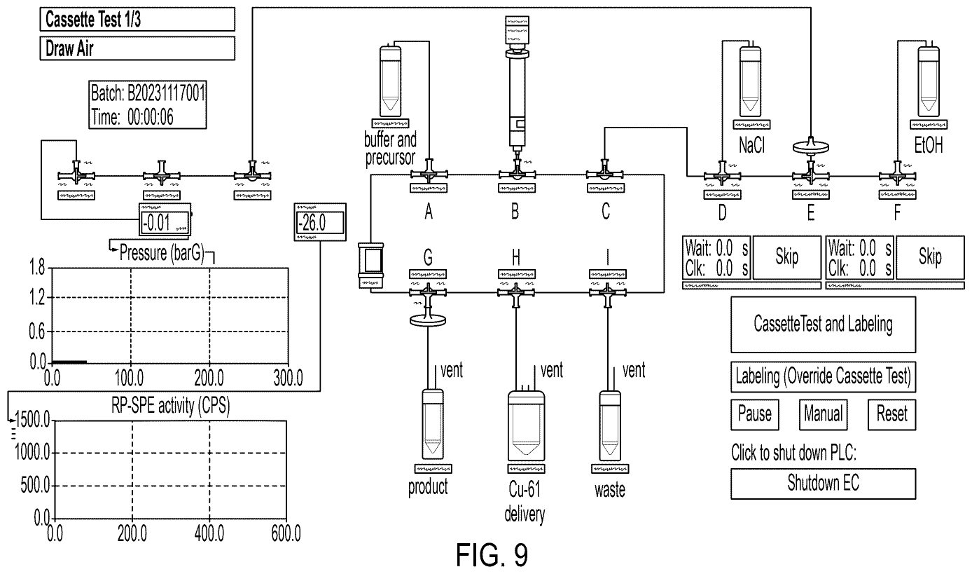

The labelling process was performed on an Eckert & Ziegler ModularLab Pharm Tracer device (FIG. 9). Cassettes and reagent kits were obtained from Eckert & Ziegler. In addition, perfusor lines and Luer m/m adapters for the modification of the cassettes were obtained from B. Braun S E. Vitamin C Streuli® from Streuli Pharma AG was used as additive for the isotonic sodium chloride solution in the reagent kit. The specifications are defined by the manufacturers of the materials listed in Table 21.

Streuli® contains 100 mg/mL ascorbic acid, 250 mg/mL propylene glycol, 14.8 mg/mL sodium bicarbonate, and 0.03 mg/mL sodium EDTA.

TABLE 21
Materials used in the labelling process for
[⁶¹Cu]Cu-NODAGA-LM3 injection solution.

| Item | content | origin |
|---|---|---|
| reagent kit | syringes, needles | Eckert & Ziegler |
| Vitamin C | and micro pins | Eckert & Ziegler |
| Streuli ® | sodium acetate trihydrate | Eckert & Ziegler |
| | water trace select | Eckert & Ziegler |
| | iso. Saline solution | n.a. |
| | ethanol/Water | Streuli Pharma AG |

The following reagent preparation was done in a sterile workbench with reagent kit materials from EZAG and Vitamin C Streuli®:

Using a 2 ml syringe with sterican canula, 1 ml Vitamin C Streuli was transferred into the vial with isotonic saline solution.

Using a 5 ml syringe with blue plastic tip, 0.9 to 1.0 ml ethanol/water mixture (50%) was transferred into the vial with water (Trace Select). The entire volume was transferred into the vial with sodium acetate trihydrate and dissolved completely.

Using a 1 ml syringe with blue plastic tip, dissolve NODGA-LM3 with 0.6-0.7 ml of the sodium acetate solution.

### Cassette Preparation

After switching on the system and initiation of the method, the cassette was installed and modified so that the lines corresponded to the scheme shown in FIG. 9. All modifications used kit materials, and additionally, one perfusor line and one Luer m/m adapter. The following procedure is used for the modification of the cassette:
Load template and press “Cassette Test & Labelling”
Place and lock cassette
Remove glass vial with line on connector A2
Empty lower row and both sides
Connect C2 with D1
Close F3 with blind plug of connector C2
Put the C-18 cartridge on connector G3 and connect it to Al
Remove the T-connector from waste line and connect latter with 12 and to a vented Falcon vial (50 ml)
Connect the remaining long line to H2 and to a sterican needle on the other end of the line
Connect a perfusor line with a male-to-male Luer adapter to G2 and to a sterile filter and a sterican needle on the other end to the product vial
Connect empty syringe (2 ml) without piston to position A2
Connect the blue line of the pressure manifold to the sterile filter on position E2

The labelling was carried out in the ⁶¹Cu delivery vial, which was used as a reactor. The labelling process started with the addition of 1:1 v/v sodium acetate solution (0.5 M) with 80 μg of NODAGA-LM3 via a metal needle to the [⁶¹Cu]Cu²⁺ labelling solution. The metal needle remained in the solution during the labelling duration of 4 min at RT. The labelling mixture was then diluted with 2 mL of isotonic sodium chloride solution with 2% v/v Vitamin C Streuli® and passed over a Waters Sep-Pak Light C18 SPE cartridge. The reactor and the RP cartridge were washed twice with 2-3 mL of the previously described sodium chloride solution. [⁶¹Cu]Cu-NODAGA-LM3 was finally eluted with 1.5 mL of an ethanol/water mixture (1:1) via a sterile filter into a product vessel, followed by 8 mL of the sodium chloride solution with Vitamin C Streuli®. As ascorbic acid, disodium EDTA and sodium bicarbonate are not retained on the RP phase of the SPE cartridge, they are part of the product formulation. The detailed process description is listed in Table 22.

TABLE 22
Process overview for the production of [⁶¹Cu]Cu-NODAGA-LM3 injection solution.

| Phase | Section | Subsection | Description |
|---|---|---|---|
| initialization | n.a. | n.a. | system settings for cassette mounting |
| pressure test | 1 pressurizing | n.a. | build pressure to around 1.3 bar |
| | 2 pressure test | n.a. | test of pressure stability |
| | 3 venting | n.a. | cassette venting |
| labelling | 1 RP-SPE cond. | 1 ethanol | conditioning of RP cartridge with 50% ethanol |
| | | transfer | purge cassette lines |
| | | 2 line purge | |
| | 2 start conditions | n.a. | system on hold for process preparation |
| | 3 labelling | 1 precursor add. | addition of precursor and buffer to ⁶¹Cu |
| | | 2 labelling time | 2 min timeout for labelling at RT |
| | | 3 RP cond. | conditioning of RP cartridge with iso. NaCl |
| | | NaCl | solution |
| | 4 purification | 1 dilution | dilution of the labelling mix with NaCl solution |
| | | 2 RP trapping | trapping of CuLM on RP SPE cartridge |
| | | 3 reactor wash | flushing of the reactor and trapping on RP |
| | | 4 RP washing | cartridge |
| | | | washing of the RP cartridge with iso. NaCl |
| | | | solution |
| | 5 formulation | 1 RP elution | elution of ⁶¹CuLM to product vial with 50% EtOH |
| | | 2 formulation | dilution of the product with iso. NaCl solution |
| | | 3 filter purge | purging of the sterile Filter and cassette venting |

Immediately after the end of the synthesis (EoS), the total activity and the product volume were measured, a sample was taken and split into the following three portions:
The sterility test sample with a volume of at least 0.5 ml was used for the retrospective sterility test.
The QC sample with a volume of at least 50 μl was used for all remaining QC tests that follow the labelling process and were needed for batch release.
The retention sample with a volume of at least 0.5 ml was needed for possible retesting purposes.

The formulation of the injected dose per patient is provided in Table 23.

TABLE 23
Composition of the [⁶¹Cu]Cu-NODAGA-LM3 solution for injection.

| Component | Typical batch | | Typical dose | | Origin |
|---|---|---|---|---|---|
| total volume | 9.5 | mL | 3.6 | mL | — |
| Isotonic saline | 8.8 | mL | 3.3 | mL | 7.3 mL 0.9% iso. NaCl |
| solution | | | | | 1.5 mL ethanol 50% |
| | | | | | 180 μL Vitamin C Streuli ® |
| Ethanol | 592 | mg | 224 | mg | 1.5 mL ethanol 50% |
| Ascorbic acid | 18 | mg | 6.8 | mg | 180 μL Vitamin C Streuli ® |
| Propylene glycol | 45.5 | mg | 17 | mg | 180 μL Vitamin C Streuli ® |
| Sodium bicarbonate | 2.7 | mg | 1 | mg | 180 μL Vitamin C Streuli ® |
| Sodium EDTA | 0.005 | mg | 0.002 | mg | 180 μL Vitamin C Streuli ® |
| NODAGA-LM3 and | 64 | μg | 24 | μg | 80 μg NODAGA-LM3 |
| [⁶¹Cu]Cu-NODAGA- | | | | | |
| LM3 | | | | | |
| [⁶¹Cu]Cu-NODAGA- | 400 | MBq | 150 | MBq | ~450 MBq ⁶¹Cu labelling |
| LM3 | | | | | solution |

The labelling yield was usually quantitative. Radiochemical yield was lowered by process losses and was usually limited to below 10% (Table 24). Other losses, up to 4%, were transfer losses on the lines of the cassette and remaining activity in the reactor.

TABLE 24
Characteristic values of the production process
for [⁶¹Cu]Cu-NODAGA-LM3 injection solution.

| Criteria (reference time at SoS) | CuLM-1 | | CuLM-2 | | CuLM-3 | |
|---|---|---|---|---|---|---|
| activity at SoS | 751 | MBq | 776 | MBq | 649 | MBq |
| processing time | 13 | min | 13 | min | 13 | min |

| losses in delivery vial (reactor) | 2.5% | 1.7% | 1.4% |
|---|---|---|---|
| RP-SPE cartridge | 0.1% | 0.1% | 0.1% |
| sterile filter | 0.9% | 0.8% | 0.6% |
| waste container | 0.7% | 1.0% | 0.5% |
| remaining losses | 1.2% | 1.8% | 1.2% |
| RLY (HPLC + Waste) | 99.2% | 99.0% | 99.5% |
| RCY | 710 MBq (94.5%) | 774 MBq (94.6%) | 624 MBq (96.2%) |
| yield | 666 MBq (88.7%) | 688 MBq (88.7%) | 587 MBq (90.5%) |

Note
“SoS” refers to Start of Synthesis.

### 5.2.2. Control of Excipients

[⁶¹Cu]⁶¹Cu-NODAGA-LM3 injection solution contains ethanol 50%, isotonic sodium chloride solution and Vitamin C Streuli as excipients (see also Table 21). All materials were obtained from a commercially available reagent set with CoA, with the exception of Vitamin C Streuli®. Ethanol 50% contains ethanol in Ph. Eur. quality and water for injection with market authorization. Isotonic sodium chloride solution has market authorization. Vitamin C Streuli® has market authorization.

### 5.2.3. Control of the Product

The release of [⁶¹Cu]Cu-NODAGA-LM3 injection solution for clinical use depends on the compliance of the QC results to the specifications listed in Table 25.

TABLE 25
Release specifications of [⁶¹Cu]Cu-NODAGA-LM3 injection solution.

| Parameter | test procedure | specification | |
|---|---|---|---|
| total activity | dose calibrator | ≥200 | MBq |
| volume | differential weighing | 9 ± 1 | mL |
| activity concentration | calculation | ≥20 | MBq/mL |
| appearance | visual | clear, slightly yellow | |
| pH value | test stripes | 5-7 | |
| ascorbic acid content | test stripes | ≥1 | mg/mL |
| ethanol content | GC | ≤10.0% | v/v |
| identity | HPLC | \| Δt_{R} \| ⁶¹CuLM to ^{nat}CuLM ≤ 15 s | |
| radiochemical purity | HPLC (radio channel) | ≥95.0% ⁶¹CuLM | |
| concentration of Cu-NODAGA-LM3 | HPLC (UV channel) | ≤5.0% ⁶¹Cu²⁺ (free) | |
| and related substances | HPLC (UV channel) | ≤12.0 μg/mL (Σ(M)LM) | |
| sum of Cu-NODAGA-LM3 and related | γ-spectrometry | 40-100 | μg |
| substances | bubble point test | ≥99.9% | |
| radionuclide purity | LAL test (Endosafe) | ≥3450 | mbar(G) |
| sterile filter integrity | Growth | ≤17.5 | EU/mL |

| | |
|---|---|
| endotoxine content | Sterile |
| sterility* | |

*retrospective release parameter

### 5.2.4. Batch Analysis

Multiple production runs of [⁶¹Cu]Cu-NODAGA-LM3 injection solution met the release specifications. The results are summarized in Table 26. [61Cu]Cu-NODAGA-LM3 was produced with an activity strength of 200 to 500 MBq, at an activity concentration of 20 to 60 MBq/mL, and with a radiochemical purity of at least 95%. It can be used for up to two patient doses.

TABLE 26
Quality control results of [⁶¹Cu]Cu-NODAGA-LM3 injection solution.

| Parameter | specification | | Batch 1 | | Batch 2 | | Batch 3 | |
|---|---|---|---|---|---|---|---|---|
| product activity | ≥200 | MBq | 666 | MBq | 693 | MBq | 588 | MBq |
| product volume | 9 ± 1 | mL | 8.7 | mL | 9.4 | mL | 9.5 | mL |
| activity concentration | ≥20 | MBq/mL | 76.6 | MBq/mL | 73.7 | MBq/mL | 61.9 | MBq/mL |
| appearance | clear, slightly yellow | | clear, colorless | | clear, colorless | | clear, colorless | |
| pH value | 5-7 | | 6.5 | | 6.5 | | 7.0 | |
| ascorbic acid content | ≥1 | mg/mL | 3 | mg/mL | 2 | mg/mL | 3 | mg/mL |
| filter integrity | ≥3450 | mbar | 3873 | mbar | 3876 | mbar | 3718 | mbar |
| ethanol content | ≤10.0% | v/v | 9.2% | v/v | 9.0% | v/v | 8.5% | v/v |
| identity | \| Δt_{R} \| ≤ 15 | s | 2 | s | 2 | s | 8 | s |
| RCP ⁶¹CuLM | ≥95.0% | | 99.5% | | 99.4% | | 99.2% | |
| ⁶¹Cu²⁺ (free) | ≤5.0% | | 0.0% | | 0.0% | | 0.0% | |
| (M)-LM concentration | ≤12 | μg/mL | 6.4 | μg/mL | 5.8 | μg/mL | 6.6 | μg/mL |
| (M)-LM content | 40-100 | μg | 56 | μg | 55 | μg | 63 | μg |
| endotoxine content | ≤17.5 | EU/mL | <5.0 | IU/mL | <5.0 | IU/mL | <5.0 | IU/mL |
| sterility* | sterile | | passed | | passed | | passed | |
| nuclide identity** | 486-538 | keV | 514 | keV | 514 | keV | 514 | keV |
| half-life time** | 190-210 | min | 201 | min | 199 | min | 201 | min |

*retrospective value
**only for validation purpose

### 5.2.4.1 Ascorbic Acid Content

The ascorbic acid concentration was around 2 mg/mL due to the addition of 1 mL Vitamin C Streuli® (10 mg ascorbic acid) to the isotonic sodium chloride vial of 50 mL. The lowest calculated concentration for ascorbic acid in the injection solution is for the highest specified product volume 1.8 mg/mL, as the total volume of 10 mL contains 1.5 mL aqueous ethanol solution. The injection solution was diluted with a factor of 1:10 with water to reduce the sample volume and thus, the exposure to radioactivity during the test. The resulting concentration was then 0.18 mg/mL.

The ascorbic acid content was checked through a semi-quantitative method via Quantofix® test strips. The strips were dipped for 10 s into this solution and developed for additional 30 s. The ascorbic acid value was multiplied by 10 to compensate the dilution factor.

### 5.2.4.2 Radiochemical Purity (RCP) by HPLC

The RCP by HPLC is the ratio of the counts due to [⁶¹Cu]Cu-NODAGA-LM3 compared to the total counts in the chromatogram. To ensure a representative value, the remaining counts, which are not included in the sum of Region of Interest (“ROI”, i.e., the peaks on the chromatogram that are integrated), must be below 1.0% of the total counts. RCP must be over 95%. In addition, the content of free ⁶¹Cu²⁺ must be under 5% of the total activity.

### Characterization of Impurities

An identified impurity was free ⁶¹Cu²⁺, which was present in the injection solution in small amounts (≤1%). Experiments with very low RLY (e.g., below 50%) showed that the reverse-phase HPLC purification during the process effectively removes the unbound ⁶¹Cu content.

The HPLC method for the analysis of [⁶¹Cu]Cu-NODAGA-LM3 was validated regarding the trueness of the result for this impurity with a spike experiment. The content of unbound ⁶¹Cu is part of the release criteria and specified with a maximum of 5%.

The HPLC setup consisted of Agilent Infinity line 2 modules with a quaternary pump, a VWD and an ALS. It also includes a Valco 10-column switcher and a Gabi Nova radio detector from Elysia-raytest. GinaX chromatography software from Elysia-raytest controls the entire system. The HPLC system was used to quantify the content of precursor and related substances, to determine the radiochemical purity and to prove identity (Table 28). The method details are listed below:
chromatographic column: ACE 3 C18, 3 μm, 150×3.0 mm (Part No. ACE-111-1503)
injection volume: ALS, 20.0 μl (1.00 μl for SST)
radio detector settings: run time 11 min, discriminator limits at 300 and 2000 keV
VWD settings: run time 11 min, 280 nm, 10 Hz
flow rate: run time 14 min, 0.80 ml/min
eluent A: water (HPLC)+0.1% TFA
eluent B: acetonitrile (HPLC)+0.1% TFA
gradient control, time (% B): initial (20); 10.0 min (45); 10.5 min (95); 11.0 min (95); 11.5 min (20).

### 5.2.4.3 Product Activity and Precursor Content

The precursor amount used for production was in the range of 64 to 96 μg. Considering approx. 20% of losses during processing, the content in the product solution ranged from 51 μg to 77 μg. Together with the specified product volume range of 8 to 10 mL, the concentration range was between 9.2 μg/mL (highest) and 5.1 μg/mL (lowest) (Table 27). V150 MBq refers to the volume equivalent to 150 MBq.

The activity dose was set to 150 MBq, so at least 200 MBq of activity was needed on Eos to compensate the losses due to decay. The average product activity was 400 MBq.

The limit concentration for batch release was 12 μg/mL, calculated as the maximum possible value with no losses.

TABLE 27
Administered amount of [⁶¹Cu]Cu-NODAGA-LM3 for a dose of 150
MBq with lowest and average activity concentration and product volume*

| A (EoS) | Volume | | Concentration | | V_{150 MBq @EoS} | Amount administered |
|---|---|---|---|---|---|---|
| 200 MBq | 8 | mL | 9.2 | μg/mL (highest) | 6.0 mL | 55 μg |
| 200 MBq | 10 | mL | 5.1 | μg/mL (lowest) | 6.0 mL | 31 μg |
| 400 MBq | 9.5 | mL | 6.7 | μg/mL (average) | 3.6 mL | 24 μg |

*Peptide content was calculated with 20% of process losses and a range for the bulk peptide amount from 64 to 96 μg.

### 5.2.4.4 Radiochemical Purity

The release limit value for the radiochemical purity of [⁶¹Cu]Cu-NODAGA-LM3 injection solution was set to 95.0% of the total radioactivity in the radio channel, following standard values in radiopharmaceutical monographs as given in Ph. Eur. 11. Thus, the limit for the amount of free ⁶¹Cu²⁺ was set to 5.0%.

### 5.2.4.5 Reference Standards or Materials

Standard solutions were used in GC and in HPLC analysis.

The GC standard for calibration and the SST was based on a commercially available ethanol reference standard (Merck, Ethanol-400, E-036-10×1.2 ML) and diluted with a procedure described in RCL-SOP307. This working solution contained a comparable amount of 1-Propanol (Sigma-Aldrich, HPLC grade, 34871-100 ML) as retention time standard for the daily check of the separation performance.

The HPLC standard for calibration was a solution of NODAGA-LM3 in acetic acid buffer (water: Riedel-de Haën, TraceSELECT®, 95305-500 ML; glacial acetic acid: Merck, 338826-100 ML; sodium acetate: Merck 71188-250G) with an addition of copper (II) chloride dihydrate (Fluka, 61175 250 g).

### Container Closure System

[⁶¹Cu]Cu-NODAGA-LM3 injection solution was produced for immediate use within 8 hours post end of synthesis (EoS). Patient doses were filled into tungsten-coated disposable syringes and stored in lead-shielded transport containers until application.

### 5.2.4.6 Stability

The product was intended to be used immediately after production, not stored for an extended period of time. The proof of stability over the shelf life was based on the RCP via HPLC. Table 28 summarized the results of a product stability assay for [⁶¹Cu]Cu-NODAGA-LM3 injection solution during a total time of 24 h, while the bulk solution was stored at room temperature. The product was safely applicable over the full shelf life of 8 h.

TABLE 28
Product stability assay for the shelf life
of [⁶¹Cu]Cu-NODAGA-LM3 injection solution.

| | | | ⁶¹Cu²⁺ | | ⁶¹CuLM |
|---|---|---|---|---|---|
| batch | time | Σ(M)-LM^{a} | (free) | impurities | (RCP) |
| 1 | EoS | 6.4 μg/mL | 0.0% | 0.6% | 99.5% |
| | EoS + 3.9 h | 5.8 μg/mL | 0.0% | 0.8% | 99.1% |
| | EoS + 7.3 h | 4.9 μg/mL | 0.1% | 0.9% | 99.0% |
| | EoS + 24 h | 4.7 μg/mL | 0.1% | 1.5% | 98.5% |
| 2 | EoS | 5.8 μg/mL | 0.0% | 0.6% | 99.4% |
| | EoS + 3.4 h | 5.2 μg/mL | 0.1% | 0.8% | 99.1% |
| | EoS + 7.3 h | 5.3 μg/mL | 0.0% | 1.1% | 99.0% |
| | EoS + 23 h | 3.7 μg/mL | 0.2% | 1.5% | 98.3% |
| 3 | EoS | 6.6 μg/mL | 0.0% | 0.8% | 99.2% |
| | EoS + 3.9 h | 5.3 μg/mL | 0.1% | 0.8% | 99.0% |
| | EoS + 19 h | 4.8 μg/mL | 0.1% | 1.4% | 98.5% |
| | EoS + 27 h | 4.6 μg/mL | 0.0% | 1.5% | 98.4% |

^{a}total amount of peptide, i.e., sum of the total peaks related to the peptide that chelated Cu or other metals

### 5.3. Example 3: In Vitro and In Vivo Studies

### 5.3.1. Lipophilicity (log D (pH 7.4))

The lipophilic/hydrophilic character of [⁶¹Cu]Cu-NODAGA-LM3 has been assessed by the determination of the distribution coefficient (D), expressed as log D at physiological pH (log D (pH 7.4)), between an aqueous and an organic phase following the “shake-flask” method. [⁶¹Cu]Cu-NODAGA-LM3 (1 μM) was added to a 50:50 pre-saturated mixture solution of 1-octanol and phosphate-buffered saline (PBS pH 7.4). The mixture was vortexed for 30 min and then centrifuged at 3000 rpm to achieve phase separation. Aliquots from each phase were collected and measured in a gamma counter. The distribution coefficient was calculated as the average logarithmic values of the ratio between the radioactivity in the organic and the PBS phase. Calculations revealed a log D (pH 7.4)=−2.95±0.08. This value does not differ from the mean of other compounds in the same class. It indicates that the molecule tends to partition into aqueous body compartments and excreted through the kidneys.

### 5.3.2. Binding Affinity (IC₅₀) to Different SSTR Subtypes

The binding affinity of Cu-NODAGA-LM3 was determined by measuring the IC₅₀ by labelling the precursor with natural copper (^{nat}Cu). The IC₅₀ value is the concentration in which 50% of the specific binding of a reference radioligand to the tested molecule is inhibited. The universal somatostatin radioligand [Leu,D-Trp,¹²⁵I-Tyr]-somatostatin-28 was used as a reference radioligand. The assay was performed on 20-mm-thick cryostat sections of membrane pellets prepared from SSTR1-, SSTR2-, SSTR3-, SSTR4-, and SSTR5-expressing cells incubated with increasing concentrations of ^{nat}Cu-NODAGA-LM3. IC₅₀ values for each SSTR subtype were calculated by mathematically extracting the concentration of the tested molecule, causing 50% displacing of the specific bounded [Leu,D-Trp,¹²⁵I-Tyr]-somatostatin-28. The results are reported in Table 29.

^{nat}Cu-NODAGA-LM3 exhibits specificity for SSTR2 (IC₅₀=6.7±1.5 nM) over the other SSTR subtypes (IC_{50≥1000} nM). These findings suggest that the investigated radiopharmaceutical is a promising candidate for the selective targeting of SSTR2-expressing tumors, leading to PET images with a high tumor-to-organ signal ratio and reduced false positives.

TABLE 29
IC₅₀ values of [^{61/64}Cu]Cu-NODAGA-LM3 for the SSTR subtypes.

| | SSTR1 | SSTR2 | SSTR3 | SSTR4 | SSTR5 |
|---|---|---|---|---|---|
| IC₅₀ (nM) | ≥1000 | 6.7 ± 1.5 | ≥1000 | ≥1000 | ≥1000 |

### 5.3.3. In Vitro Cellular Uptake, Internalization and Dissociation

The cellular uptake and internalization of [⁶⁴Cu]Cu-NODAGA-LM3 were studied in vitro on SSTR-expressing cells, HEK-SST2. The cells were seeded in 6-well plates overnight, then washed and incubated at 37° C. with [⁶⁴Cu]Cu-NODAGA-LM3 (0.25 pmol/mL in cell DMEM medium). The internalization was interrupted at different time points by removing the unbound [⁶⁴Cu]Cu-NODAGA-LM3 by washing three times with ice-cold PBS. Then, cells were treated for 5 min with ice-cold glycine solution (0.05 M, pH 2.8) to detach the cell surface-bound radiotracer. Afterwards, the cells containing the internalized [⁶⁴Cu]Cu-NODAGA-LM3 were detached with 1 M sodium hydroxide. All the fractions were collected and measured separately via a gamma counter. The results are presented in Table 30.

TABLE 30
In vitro surface-bound and internalized [⁶⁴Cu]Cu-
NODAGA-LM3 on HEK-SST₂ cells. Results are presented
as mean value ± standard deviation (n = 3).

| | |
|---|---|
| Timepoint | Cell-associated fraction [%] |

| [min] | Surface-bound | Internalized |
|---|---|---|
| 30 | 27.4 ± 0.4 | 2.7 ± 0.5 |
| 60 | 33.0 ± 6.0 | 4.6 ± 0.3 |
| 120 | 37.4 ± 4.2 | 7.3 ± 0.2 |
| 240 | 37.2 ± 4.9 | 12.1 ± 0.2 |

Dissociation experiments were performed, incubating the HEK-SST2 cells with [⁶⁴Cu]Cu-NODAGA-LM3 (0.25 pmol/mL in cell DMEM medium) for 2 hours at 4° C. The cells were then washed in cold PBS and left in the medium at 37° C. for up to 4 hours. At different time points, the medium was collected for measurement via a gamma counter. Non-specific binding and internalization were determined using a 1000-fold excess of DOTA-LM3. The specific cell surface-bound, internalized, and dissociated radiotracer amounts were expressed as a percentage of the total applied activity after subtracting the non-specific values. The results are presented in Table 31.

TABLE 31
Fate of the surface-bound [⁶⁴Cu]Cu-NODAGA-LM3 after
incubation at 37° C. in DMEM medium. Results
are presented as mean value ± standard deviation (n = 3).

| | |
|---|---|
| Timepoint | Cell-associated fraction [%] |

| [min] | Surface-bound | Internalized | Free |
|---|---|---|---|
| 10 | 79.7 ± 4.0 | 1.2 ± 0.1 | 12.3 ± 0.8 |
| 20 | 70.9 ± 0.3 | 5.2 ± 1.2 | 19.0 ± 0.1 |
| 30 | 58.3 ± 4.5 | 4.5 ± 0.6 | 21.6 ± 1.6 |
| 60 | 62.0 ± 6.2 | 6.9 ± 0.3 | 25.9 ± 1.9 |
| 120 | 52.4 ± 5.7 | 12.1 ± 0.6 | 25.0 ± 1.4 |
| 240 | 50.9 ± 2.4 | 15.6 ± 0.6 | 26.1 ± 0.8 |

The results highlight a fast uptake on the cellular membrane, superior if compared to STTR2 agonist-based radiopharmaceuticals and in line with the other molecules in the same class. The lack of significant internalization is attributed to the inability of SSTR2 antagonists to induce the conformational changes necessary for the activation of internalization processes. [⁶⁴Cu]Cu-NODAGA-LM3 is retained on the receptor, levelling off after 4 hours at approximately 55%. Within 4 hours, 16% of the receptor-bound activity was internalized, and 26% was released in the DMEM medium.

### 5.3.4. Pharmacokinetics and Product Metabolism in Animals

Unless otherwise specified, the in vivo studies were performed on athymic nude Foxn1^{nu} 4-6 weeks-old mice. Animals were injected in the shoulder with HEK-SST2 cells (10⁷ cells/200 μL) suspended in PBS. The in vivo experiments were performed when tumors reached a volume between 100-500 mm. At the intervention, all mice were between 7 and 11 weeks old. All animal experiments were performed following the guidelines for using living animals in scientific studies and the Swiss regulations for the protection of animals.

### 5.3.5. Biodistribution Studies

Quantitative biodistribution studies were performed on HEK-SST2 xenografted mice to determine the distribution of [⁶¹Cu]Cu-NODAGA-LM3 following i.v. injection into the tail vein. The dose of the administered radiopharmaceutical was 1 MBq (20 μmol) diluted to 100 μL with saline solution. The mice were euthanized at 4 hours post-injection, and the organs of interest were collected, rinsed, blotted, weighed, and analyzed in a gamma counter. The results were expressed as the percentage of injected activity per gram of tissue (% IA/g) and reported as mean±standard deviation in Table 32. Tumor-to-organ ratios were calculated and reported in Table 33.

The highest uptake was measured in the SSTR2-expressing tumor (19.0±2.1% IA/g at 4 hours post-injection) and is attributed to the high affinity and selectivity of the investigated radiopharmaceutical for SSTR2. At 4 hours post-injection, the tumor-to-organ uptake ratio was higher than 5 for all the organs except for kidneys, which exhibited an uptake of 3.87±0.51% IA/g due to the excretion pathway. The pituitary gland had a relatively high uptake compared to the other organs, which was attributed to the specific expression of SSTR subtypes 1, 2, and 5 in this organ. Lower uptake was registered for blood, muscle and spleen. From a pharmacokinetic perspective, these results suggest that [⁶¹Cu]Cu-NODAGA-LM3 had a fast clearance from the bloodstream and was rapidly excreted through the kidneys. The [⁶¹Cu]Cu-NODAGA-LM3 clearance from the body was further investigated in pharmacokinetic studies. High specificity was further investigated by blocking studies.

TABLE 32
Biodistribution of [⁶¹Cu]Cu-NODAGA-LM3
HEK-STT₂ xenografted mice at 4 hours post-injection.
Results are expressed as % IA/g ± standard deviation.

| Organ | Uptake [% IA/g] |
|---|---|
| Blood | 0.11 ± 0.01 |
| Heart | 0.17 ± 0.02 |
| Lung | 0.42 ± 0.05 |
| Liver | 0.73 ± 0.06 |
| Pancreas | 0.44 ± 0.24 |
| Spleen | 0.18 ± 0.02 |
| Stomach | 0.79 ± 0.13 |
| Intestine | 0.58 ± 0.12 |
| Adrenal | 0.61 ± 0.18 |
| Kidneys | 3.87 ± 0.51 |
| Muscle | 0.10 ± 0.02 |
| Bone | 0.42 ± 0.12 |
| Pituitary | 3.64 ± 0.31 |
| HEK-STT₂-tumor | 19.0 ± 2.10 |

TABLE 33
Tumor-to-organ uptake ratios of [⁶¹Cu]Cu-NODAGA-LM3
in HEK-STT₂ xenografted mice at 4 hours post-injection.

| Tumor/Organ | Ratio |
|---|---|
| Tumor/Blood | 181.0 |
| Tumor/Liver | 26.0 |
| Tumor/Kidney | 4.9 |
| Tumor/Pituitary | 5.3 |
| Tumor/Muscles | 185.0 |

### 5.3.6. Pharmacokinetics Studies

The pharmacokinetics of the investigated radiopharmaceutical was evaluated in female HEK-SST2 xenografted mice up to 24 hours post-injection. Mice were injected in the tail vein with 0.15 MBq (10 μmol) of [^{61/64}Cu]Cu-NODAGA-LM3 diluted to 100 μL with saline solution. The biodistribution was performed at 1, 4 and 24 hours, using seven animals per group. The results are presented in Table 34 and are expressed as mean±standard deviation.

In vivo, the investigated radiotracer underwent rapid renal excretion within the first 4 hours post-injection, primarily due to its hydrophilic nature and low non-specific binding in healthy tissues. The maximum accumulation in SSTR2-expressing tumor tissues was reached at 4 hours post-injection (37.88±3.43% IA/g), significantly increasing the tumor-to-organ uptake ratio. In this context, the detection ratio of malignant lesions can be improved via delayed time point PET imaging. The hepatic excretion route, often associated with the presence of free copper isotopes or metabolites, was marginal and lower when compared with the radiopharmaceuticals of the same class. The radiopharmaceutical accumulated in the liver was almost completely washed out 4 hours post-injection. The same trend was detected for the stomach and pancreas, which commonly show high uptake due to specific binding. This trend indicated a quick efflux rate and a reduced burden for the patients in these SSTR2-expressing tissues.

TABLE 34
Biodistribution data of [^{61/64}Cu]Cu-NODAGA-LM3 on healthy
mice at 1, 4, and 24 hours post-injection. Values are
presented as the mean of % AI/g ± standard deviation.

| Organ | 1 hour | 4 hours | 24 hours |
|---|---|---|---|
| Blood | 0.84 ± 0.10 | 0.11 ± 0.05 | 0.12 ± 0.05 |
| Heart | 0.54 ± 0.08 | 0.14 ± 0.07 | 0.19 ± 0.08 |
| Liver | 2.23 ± 0.48 | 0.96 ± 0.14 | 0.78 ± 0.15 |
| Spleen | 0.94 ± 0.21 | 0.21 ± 0.10 | 0.19 ± 0.10 |
| Lung | 5.89 ± 1.51 | 0.60 ± 0.17 | 0.54 ± 0.13 |
| Kidney | 10.90 ± 1.10 | 4.41 ± 0.74 | 1.25 ± 0.47† |
| Stomach | 9.17 ± 2.49 | 1.31 ± 0.55 | 0.46 ± 0.17 |
| Intestine | 1.35 ± 0.12 | 0.46 ± 0.04 | 0.33 ± 0.06 |
| Adrenal | 0.73 ± 0.15 | 0.02 ± 0.01 | 0.02 ± 0.01 |
| Pancreas | 16.26 ± 4.04 | 0.55 ± 0.10 | 0.23 ± 0.09 |
| Muscles | 0.20 ± 0.03 | 0.03 ± 0.01 | 0.02 ± 0.01 |
| Bone | 2.23 ± 1.01 | 0.39 ± 0.07 | 0.30 ± 0.16 |
| Tumor | 35.46 ± 5.70 | 37.88 ± 3.43 | 14.79 ± 2.40 |

### 5.3.7. PET/CT Imaging

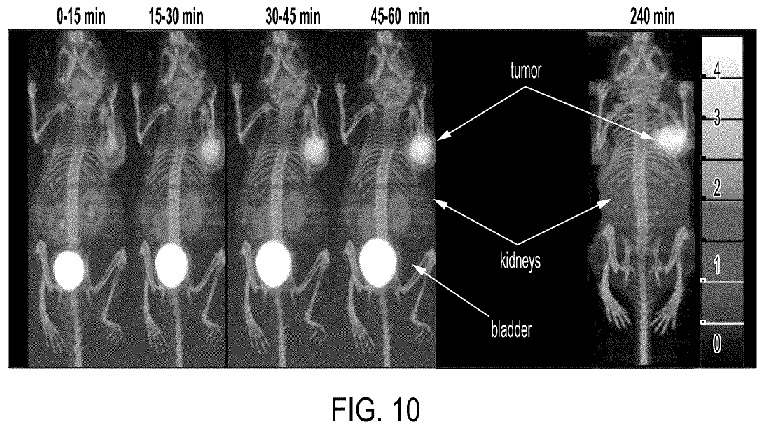

HEK-SST₂ xenografted mice were injected into the tail vein with 13 MBq (200 μmol) of [⁶¹Cu]Cu-NODAGA-LM3 diluted in 100 μL saline solution. Dynamic PET scans were acquired for 1 hour post-injection of the investigated radiopharmaceutical. The mice were euthanized using carbon dioxide at 4 hours post-injection, the bladder was mechanically emptied, and static PET scans were acquired for 30 min. PET images were obtained using the β-CUBE PET scanner system (MOLECUBES) and were decay corrected and reconstructed with the VivoQuant software version 4.0. Tomograms and helical CT scans of the whole mouse were first acquired using a NanoSPECT/CTTM scanner (Bioscan Inc.). CT images were reconstructed using CTReco (version r1.146), resulting in a pixel size of 0.2 mm. InVivoScope (version 1.43, Bioscan Inc.) software was used to generate the maximum intensity projection (MIP) of the co-registered PET/CT images presented in FIG. 10.

The images show the quick accumulation of [⁶¹Cu]Cu-NODAGA-LM3 in the tumor, reaching the maximum at 4 hours post-injection. The constant washout of [⁶¹Cu]Cu-NODAGA-LM3 from off-target body compartments led to a consistent decrease of the background signal, particularly evident in the dynamic scan. The optimal tumor-to-background signal ratio was reached in the scan at 4 hours post-injection. The results demonstrate the feasibility of using [⁶¹Cu]Cu-NODAGA-LM3 delayed time point imaging to enhance contrast. The delayed time point imaging impacts the overall quality of the PET image, allowing for the visualization of malignant lesions that might have gone unnoticed with conventional SSTR2-PET/CT agonist radiotracers, e.g., NET tumors with reduced expression of SSTR2 or lymph nodes and distant metastases in areas characterized by high background signal due to specific or off-target binding, such as liver, pancreas, and guts. Therefore, using [⁶¹Cu]Cu-NODAGA-LM3 may increase the tumor detection ratio.

### 5.3.8. Specificity Study

The specificity study was performed parallel to pharmacokinetics studies. For this reason, NODAGA-LM3 was radiolabeled with ⁶⁴Cu.

The specificity of [⁶⁴Cu]Cu-NODAGA-LM3 for SSTR2-expressing tissues was evaluated in HEK-SST2 xenograft mice through a blocking study. The mice were administered with 0.15 MBq of [⁶⁴Cu]Cu-NODAGA-LM3 co-injected with a 2000-fold excess of DOTA-LM3 as a blocking agent. The mice were sacrificed 4 hours post-injection, and the organs were collected. weighed, and counted in a gamma counter. The results, expressed as % IA/g, are presented in Table 35 compared to the results obtained from biodistribution studies.

The most significant blocking effect can be seen in the tumor (37.88±3.43% IA/g in the non-blocking group vs 0.52±0.18% IA/g in the blocking group). A blocking effect was also observed for other SSTR2-positive tissues, albeit to a lesser extent:

Bones presented a reduction from 0.39±0.07% IA/g to 0.01±0.01% IA/g. The observed specificity was likely driven by the high expression of SSTR2 on osteoblastic processes.

Specific uptake in the stomach (from 1.31±0.55% IA/g to 0.43±0.14% IA/g) was possibly due to the SSTR2 expression in the enterochromaffin-like (ECL) cells of the gastric mucosa.

In the case of the pancreas, a minor blocking effect was observed (from 0.55±0.10% IA/g to 0.12±0.03% IA/g). While the mechanism behind this effect is not yet fully understood, it may be related to its exocrine function combined with the SSTR2 expression in human β- and α-cells.

These findings demonstrate that the radiopharmaceutical binding was driven by high affinity for SSTR2. High selectivity ensured a reduced dose load for the off-target organs, warranting an enhanced safety profile in clinical settings. The uptake in the kidneys was not subjected to a significant blocking effect, confirming that the accumulation is not receptor-mediated but only due to renal excretion. This observation is consistent with the known biodistribution of this class of PET radiotracers. Therefore, kidney uptake could be a matter of concern as it may lead to a higher potential radioactive dose for patients. The organ dose was further investigated by dosimetry calculations. Liver accumulation remained stable at low levels, indicating that only a small portion of the radiopharmaceutical metabolites underwent hepatic excretion. The PET/CT imaging performed after [⁶¹Cu]CuCl₂ injection confirmed that free isotopes were excreted mainly through the liver. The accumulation in the liver was probably due to the coordination by blood proteins metabolized through the liver as ceruloplasmin, metallothionein and Copper Transport Regulator 1 (CTR1). Therefore, the investigated radiopharmaceutical was generally stable in vivo, with limited transchelation phenomena.

TABLE 35
Specificity of [⁶¹Cu]Cu-NODAGA-LM3 in LNCaP xenografts
at 4 hours post-injection. Results are expressed
as the mean of the % IA/g ± standard deviation.

| Organ | 4 hours | 4 hours blocking |
|---|---|---|
| Blood | 0.11 ± 0.05 | 0.10 ± 0.02 |
| Heart | 0.14 ± 0.07 | 0.20 ± 0.01 |
| Liver | 0.96 ± 0.14 | 0.90 ± 0.20 |
| Spleen | 0.21 ± 0.10 | 0.21 ± 0.06 |
| Lung | 0.60 ± 0.17 | 0.67 ± 0.25 |
| Kidney | 4.41 ± 0.74 | 3.52 ± 0.93 |
| Stomach | 1.31 ± 0.55 | 0.43 ± 0.14 |
| Intestine | 0.46 ± 0.04 | 0.46 ± 0.01 |
| Adrenal | 0.02 ± 0.01 | 0.22 ± 0.05 |
| Pancreas | 0.55 ± 0.10 | 0.12 ± 0.03 |
| Muscle | 0.03 ± 0.01 | 0.02 ± 0.01 |
| Bone | 0.39 ± 0.07 | 0.01 ± 0.01 |
| Tumor | 37.88 ± 3.43 | 0.52 ± 0.18 |

### 5.3.9. Dosimetry Calculations

The data from pharmacokinetic studies (Table 35) was decay corrected using the half-life of ⁶¹Cu to extrapolate the human dose. There were no measurements of the radioactivity accumulation in red marrow; this was estimated assuming a linear relationship between the blood and the red marrow residence times. The proportionality factor was the ratio between red marrow and human blood mass. The residence time (RT) for the stomach was approximated to be equal to the RT of the stomach wall since the animals were subjected to fasting before the biodistribution experiments. The heart was emptied of blood before measurements, and it was assumed that the total measured heart uptake was associated with the heart wall. OLINDA/EXM 1.0 was used to integrate the fitted time-activity curves and to estimate the organ and effective doses using the standard whole-body adult female phantom model. Since the pituitary is not included in the phantom, an extra calculation was performed, assuming the weight of the human pituitary to be 0.6 g. The results are summarized in Table 36.

The organs expressing SSTR2 and involved in the excretion routes receive the highest absorbed dose. The highest values are observed in the pancreas (1.03E-01 mGy/MBq), followed by the kidneys, liver, and stomach wall. The dose absorbed by the other organs is relatively low, with the brain and thyroid having the lowest values.

The absorbed dose in the pancreas is within the acceptable range for human application. An injected dose of 150 MBq [⁶¹Cu]Cu-NODAGA-LM3 would result in a 0.015 Gy absorbed dose, more than a thousand-fold lower than the critical threshold of 20 Gy.

The effective dose (whole-body) was estimated to be 5.73E-03 mSv/MBq, which is lower than the effective dose of:
clinically established SSTR2-targeting PET radiotracers, such as [⁶⁸Ga]Ga-DOTATOC (2.1E-02 mSv/MBq) and [⁶⁸Ga]Ga-DOTATATE (2.6 E-02-2.1E-02 mSv/MBq);
⁶⁴Cu radiolabeled SSTR2 agonist-based PET radiotracers, such as [⁶⁴Cu]Cu-SAR-TATE (4.5E-02 mSv/MBq) and [⁶⁴Cu]Cu-DOTATATE (3.2E-02 mSv/MBq); and
other SSTR2 antagonist-based PET radiotracers, such as [⁶⁸Ga]Ga-DOTA-JR11 (2.6E-02 mSv/MBq) and [⁶⁸Ga]Ga-NODAGA-JR11 (2.4E-02 mSv/MBq).

Regarding gender differences, the absorbed dose was generally higher in males than in females. This difference in absorbed dose may be due to differences in body composition, such as organ size and tissue density, which can affect the distribution and clearance of the radiotracer. The method used for dosimetry is limited by its underlying assumption of conserved biodistribution between human and mouse models.

TABLE 36
Total Absorbed Doses in different organs of [⁶¹Cu]Cu-NODAGA-
LM3. Results are expressed as mGy/MBq, if not differently specified.

| | | | | Total Absorbed |
|---|---|---|---|---|
| Target Organ | Alpha | Beta | Photon | Doses (mGy/MBq) |
| Adrenals | 0.00E+00 | 1.50E−03 | 3.89E−03 | 5.39E−03 |
| Brain | 0.00E+00 | 0.00E+00 | 1.59E−05 | 1.59E−05 |
| Breasts | 0.00E+00 | 0.00E+00 | 4.15E−04 | 4.15E−04 |
| Gallbladder Wall | 0.00E+00 | 0.00E+00 | 3.52E−03 | 3.52E−03 |
| LLI Wall | 0.00E+00 | 0.00E+00 | 8.50E−04 | 8.50E−04 |
| Small Intestine | 0.00E+00 | 8.57E−03 | 2.51E−03 | 1.11E−02 |
| Stomach Wall | 0.00E+00 | 6.83E−03 | 4.19E−03 | 1.10E−02 |
| ULI Wall | 0.00E+00 | 0.00E+00 | 2.34E−03 | 2.34E−03 |
| Heart Wall | 0.00E+00 | 9.73E−04 | 1.39E−03 | 2.37E−03 |
| Kidneys | 0.00E+00 | 5.32E−02 | 1.17E−02 | 6.49E−02 |
| Liver | 0.00E+00 | 8.14E−03 | 4.49E−03 | 1.26E−02 |
| Lungs | 0.00E+00 | 2.61E−03 | 1.23E−03 | 3.85E−03 |
| Muscle | 0.00E+00 | 0.00E+00 | 7.08E−04 | 7.08E−04 |
| Ovaries | 0.00E+00 | 0.00E+00 | 1.20E−03 | 1.20E−03 |
| Pancreas | 0.00E+00 | 8.79E−02 | 1.50E−02 | 1.03E−01 |
| Red Marrow | 0.00E+00 | 5.16E−05 | 9.35E−04 | 9.86E−04 |
| Osteogenic Cells | 0.00E+00 | 4.77E−05 | 5.77E−04 | 6.25E−04 |
| Skin | 0.00E+00 | 0.00E+00 | 2.98E−04 | 2.98E−04 |
| Spleen | 0.00E+00 | 2.14E−03 | 3.84E−03 | 5.98E−03 |
| Thymus | 0.00E+00 | 0.00E+00 | 3.85E−04 | 3.85E−04 |
| Thyroid | 0.00E+00 | 0.00E+00 | 9.46E−05 | 9.46E−05 |
| Urinary Bladder Wall | 0.00E+00 | 0.00E+00 | 3.65E−04 | 3.65E−04 |
| Uterus | 0.00E+00 | 0.00E+00 | 1.06E−03 | 1.06E−03 |
| Total Body | 0.00E+00 | 7.50E−04 | 8.71E−04 | 1.62E−03 |

### 5.3.10. Toxicology—Non-Clinical Safety Studies

The precursor-induced toxicity was evaluated using the non-radiolabeled precursor in line with the IAEA and EMA guidelines. The non-clinical safety study was performed using the microdose approach I described in the ICH guideline M3 (R2). The study was designed as an extended single-dose toxicity study in one rodent species and performed under GLP conditions. NODAGA-LM3 toxicity was evaluated after intravenous administration on day 1 of the study, followed by an observation period of 14 days. 60 (30 males and 30 females) C57BL/6NCrl mice were divided into one dose group and one control group. The test item was administered once by i.v, administration into the tail vein.

In each of the two groups, 20 male and 20 female main animals were euthanized the day after dosing, whereas 10 male and 10 female recovery animals were kept for an observation period of 14 days after dosing to detect possible delayed occurrence or persistence of, or recovery from toxic effects. Three extra mice per sex were included as replacement animals. The dose group animals were injected with a 5 mg/kg vector in a 5 mL/kg dose volume. The control group underwent injection of the vehicle alone (5 mL/kg). The animals were examined twice daily for toxicity and morbidity/mortality signs. All animals were sacrificed for necropsy. Blood was taken before necropsy for analysis of hematologic and clinical biochemistry parameters. Organs were collected, weighted, and preserved in neutral buffered formaldehyde (4%) for histopathological examination. The precursor was clinically well-tolerated, did not cause any adverse systemic or local effects, and there was no indication of delayed toxicity. There was no treatment-related mortality or any acute adverse effect registered. No effects on body weight gain or food consumption were noted. Corresponding histopathological findings did not accompany variations in the organ's weights. Therefore, these differences were considered to be unrelated to the test substance. No macroscopic or microscopic findings that could be attributed to treatment with the test substance were observed. The dose injected appears to be associated with increased white cell count in male mice. All these changes were transitory and considered non-adverse.

The results of this study establish a no adverse effect level (NOAEL) for the investigated radiopharmaceutical at 5 mg/kg. The intended maximum dose for patients is 0.8 μg/kg, which leads, considering the dose conversion factor, to a safety ratio higher than 1,000 times.

### 5.4. Example 4: Human Clinical Trial for [⁶¹Cu]Cu-NODAGA-LM3

### 5.4.1. Objective(s)

Phase I/II (n=22+5)

### Primary Study Objectives:

1. To assess the safety of [⁶¹Cu]Cu-NODAGA-LM3 in patients with well differentiated bronchopulmonary (BP) and gastroenteropancreatic (GEP) neuroendocrine tumors (NET).
2. To assess if the sensitivity of [⁶¹Cu]Cu-NODAGA-LM3 is not inferior to the standard of care [⁶⁸Ga]Ga-DOTA-TOC PET/CT in the same patients with well-differentiated BP and GEP NET (both scans at 1 h p.i.).

### Secondary Study Objectives:

1. To assess if the sensitivity of [⁶¹Cu]Cu-NODAGA-LM3 is superior to the standard of care [⁶⁸Ga]Ga-DOTA-TOC PET/CT in the same patients with well-differentiated BP and GEP NET.
2. To assess if the sensitivity of [⁶¹Cu]Cu-NODAGA-LM3 PET/CT scans acquired 1 h p.i. differs from the one of 3 h p.i. scans.
3. To assess and compare the positive predictive value (PPV) of [⁶¹Cu]Cu-NODAGA-LM3 and [⁶⁸Ga]Ga-DOTA-TOC PET/CT in the same patients with well-differentiated BP and GEP NET.
4. To determine biodistribution, pharmacokinetics and dosimetry of [⁶¹Cu]Cu-NODAGA-LM3 (6 patients).
5. To compare tumor-to-background uptake ratios (matched-pairs of lesions) of [⁶¹Cu]Cu-NODAGA-LM3 PET/CT at ˜1 and ˜3 h p.i. with [⁶⁸Ga]Ga-DOTA-TOC PET/CT at 1 h p.i.
6. To determine optimal imaging time window for acquiring [⁶¹Cu]Cu-NODAGA-LM3 PET/CT (1 vs 3 h p.i).
7. To assess and compare the interreader variability for [⁶¹Cu]Cu-NODAGA-LM3 PET/CT (1 vs 3 h p.i) and [⁶⁸Ga]Ga-DOTA-TOC PET/CT.

### Exploratory Objectives:

1. To determine the potential impact of [⁶¹Cu]Cu-NODAGA-LM3 PET/CT on the clinical management of patients with BP and GEP NET.
2. To correlate tumor and organ uptake visible on [⁶¹Cu]Cu-NODAGA-LM3 PET/CT and on quantitated post-treatment SPECT/CT after targeted radionuclide therapies.
3. To determine optimal injected activity of [⁶¹Cu]Cu-NODAGA-LM3.
4. To assess the tumor detection rate of ⁶¹Cu-NODAGA-LM3 PET/CT in ⁶⁸Ga-DOTATOC PET/CT negative but NETest-positive NET patients (max 5 additional patients).

### 5.4.2. Outcomes

### Primary Outcomes:

1. Safety: Adverse events and potential adverse drug reactions (graded as per CTCAE v5), vital signs and safety laboratory parameters (blood cell count, electrolytes, renal and liver parameters) will be repeatedly monitored before and after injection up to ˜4 h post-injection in an outpatient way. No formal sample size calculation.
2. Sensitivity of [⁶¹Cu]Cu-NODAGA-LM3 PET/CT acquired ˜1 h in comparison with that of the standard of care [⁶⁸Ga]Ga-DOTA-TOC PET/CT acquired ˜1 h p.i. (independent blinded scan interpretation). For this co-primary endpoint, a sample size calculation is provided.

### Secondary Outcomes:

1. Positive predictive value of [⁶¹Cu]Cu-NODAGA-LM3 PET/CT acquired ˜1 h and ˜3 h p.i. in comparison with [⁶⁸Ga]Ga-DOTA-TOC PET/CT acquired ˜1 h p.i. in the same patients (independent blinded scan interpretation).
2. Biodistribution, Pharmacokinetics, and Dosimetry of [⁶¹Cu]Cu-NODAGA-LM3:
   Tracer uptake distribution in organs will be assessed visually and quantitatively (organ SUVmax, SUVpeak and SUVmean) on [⁶¹Cu]Cu-NODAGA-LM3 PET/CT scans acquired at ˜1 h and ˜3 h pi;
   The maximum concentration (Cmax), blood clearance and area under the curve (AUC) of [⁶¹Cu]Cu-NODAGA-LM3 will be determined by serial blood sampling up to max. ˜18 h in 6/27 patients.
   Dosimetry of [⁶¹Cu]Cu-NODAGA-LM3: Whole body and healthy organ absorbed dose (Gy) will be determined in 6/27 patients by acquiring 3 time-point PET/CT imaging (˜1 h, ˜3 and ˜18 h pi).
3. Median of the mean tumor uptake (SUVmax) on [⁶¹Cu]Cu-NODAGA-LM3 PET/CT at the best time-point for imaging and comparison with [⁶⁸Ga]Ga-DOTA-TOC PET/CT 1 h p.i in the same patient (matched lesions only).
4. Median of the mean tumor-to-background uptake ratios (TBR) on [⁶¹Cu]Cu-NODAGA-LM3 PET/CT at the best time-point for imaging in comparison with [⁶⁸Ga]Ga-DOTA-TOC PET/CT 1 h p.i in the same patients (matched lesions only).
5. Optimal imaging time point for [⁶¹Cu]Cu-NODAGA-LM3 PET/CT:
   Tumor detection rate of [⁶¹Cu]Cu-NODAGA-LM3 PET/CT performed ˜1 h, ˜3 h post-injection.
   Mean TBR of [⁶¹Cu]Cu-NODAGA-LM3 PET/CT performed ˜1 h and ˜3 h post-injection (matched lesions only).
   Patient's preference will be integrated by using a stress thermometer asking the patient to score their experience between the injection and scanning, and during scanning on a visual analogue scale.

### Exploratory Outcomes:

1. Optimal injected activity of [⁶¹Cu]Cu-NODAGA-LM3:
   The optimal [⁶¹Cu]Cu-NODAGA-LM3 activity will be determined by reconstructing the list-mode PET data for different acquisition frame duration. Signal-to-noise ratios will be calculated for lung, pancreas, liver and small intestine as well as for at least one tumor lesion (matched lesions only).
   Detection rate of [⁶¹Cu]Cu-NODAGA-LM3 PET/CT in [⁶⁸Ga]Ga-DOTATOC PET/CT negative but NETest-positive NET patients (max 5 additional patients).
   Correlation between [⁶¹Cu]Cu-NODAGA-LM3 PET/CT and quantitated post-treatment SPECT/CT will be attempted in patients scheduled for targeted radionuclide therapies (max 10/27 patients).
   Impact on management of [⁶¹Cu]Cu-NODAGA-LM3 PET/CT in SSTSST2-positive tumors (analysis of pre-defined key imaging findings that may change the intended clinical management by tumor board decision).

### 5.4.3. Study Design

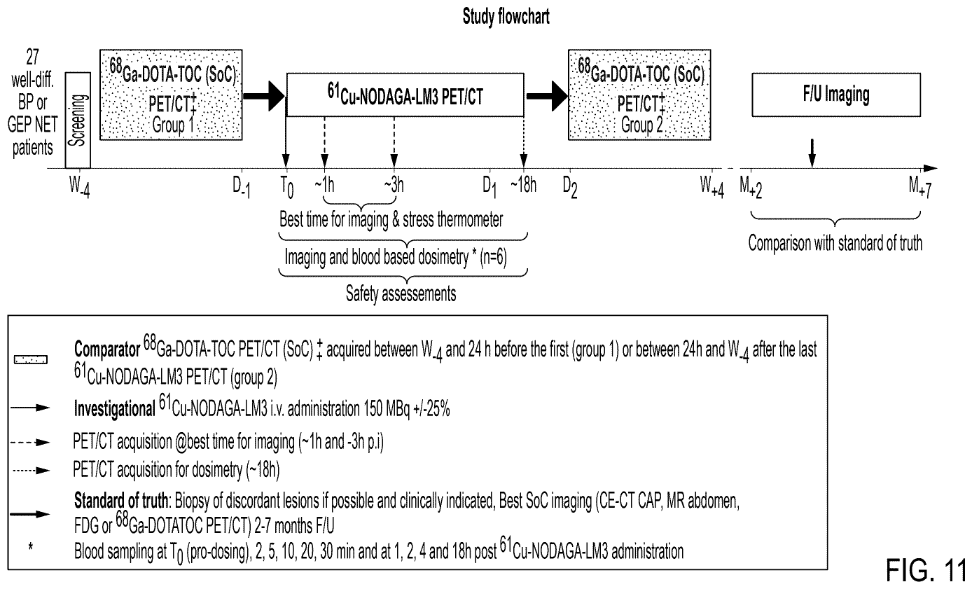

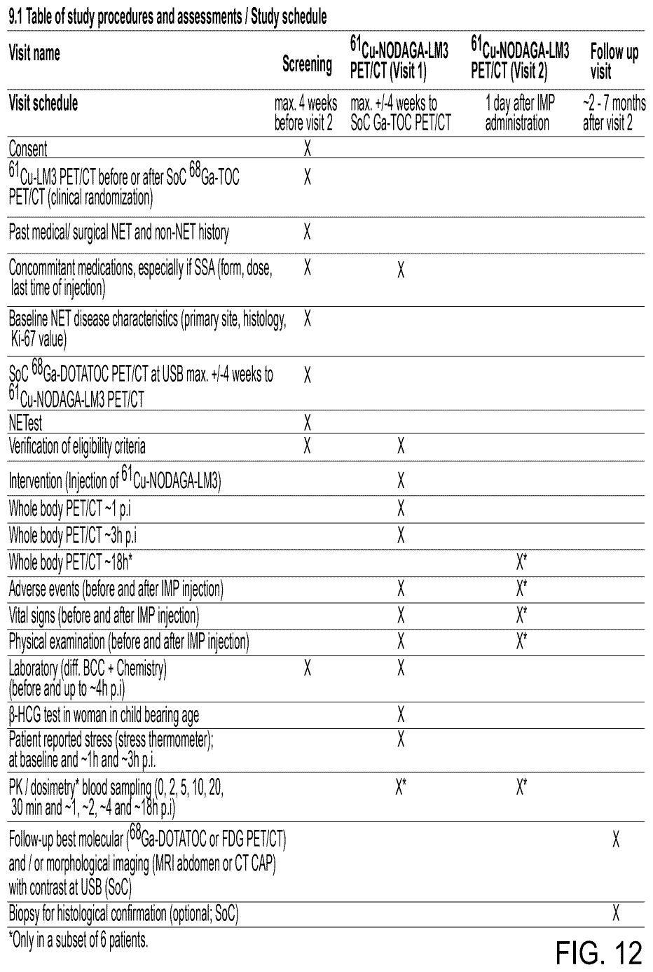

The study will be a prospective, open-label, controlled, reader-blind, single center, phase I/II PET/CT study. An illustration of the study is provided in FIG. 11.

The study product will be a single intravenous administration of GMP produced [⁶¹Cu]Cu-NODAGA-LM3 followed by up to three PET/CT acquisitions. [⁶¹Cu]Cu-NODAGA-LM3 will be administered once intravenously at an amount of 20-40 μg (or 13-26 nmol) and an activity range of 150 MBq (±25%). Imaging Schedule is summarized in Table 37.

TABLE 37
Imaging Schedule

| | | | | |
|---|---|---|---|---|
| | Screening | ⁶¹Cu-NODAGA- | ⁶¹Cu-NODAGA- | Follow up visit |
| | (max 4 weeks | LM3 PET/CT | LM3 PET/CT | (~2-7 months |
| Visit name | before visit 2) | (Visit 1) | (Visit 2) | after visit 2) |

| | | |
|---|---|---|
| Intervention | X | |
| (Injection of ⁶¹Cu- | | |
| NODAGA-LM3) | | |
| Whole body PET/ | X | |
| CT ~1 h p.i. | | |
| Whole body PET/ | X | |
| CT ~3 h p.i. | | |
| Whole body PET/ | | X |
| CT ~18 h p.i. | | |

The control intervention will be [⁶⁸Ga]Ga-DOTA-TOC PET/CT (150 MBq (±25%)) (including CE-CT), is acquired as standard of care and not part of the study, but scan results will be used as comparator for the diagnostic efficacy analysis (Phase II).

The diagnostic efficacy analysis (sensitivity, tumor uptake and image contrast (TBR), will be done in comparison with [⁶⁸Ga]Ga-DOTA-TOC PET/CT acquired as standard of care in a randomized, cross-over order. Readers of the scans will be blinded for imaging time point, tracers and clinical data and will follow a locked-read methodology. Number of Participants and Rationale: n=27 patients (including 22 patients for the sensitivity analysis powered for both superiority and non-inferiority design and 5 patients with negative [⁶⁸Ga]Ga-DOTATOC PET/CT and a positive NETest)

### Inclusion and Exclusion Criteria

Written informed consent signed
≥18 years old patients of either gender
For women in child-bearing age: a negative pregnancy test is required.
Histologically proven well-differentiated bronchopulmonary (typical or atypical carcinoid) or gastroenteropancreatic NET of all grade (including NET G3 with Ki-67 <50%).
Clinical indication to SST PET/CT imaging for either primary staging, restaging, patient selection to PRRT, treatment planning or treatment response assessment.
Standard of care [⁶⁸Ga]Ga-DOTATOC PET/CT performed or planned within max. 4 weeks prior or after IMP-administration, as clinically indicated.
At least 2 lesions detected by the previous somatostatin receptor scan, or if [⁶⁸Ga]Ga-DOTATOC PET/CT is negative, a positive NETest not older than 4 weeks should be available in 5 additional patients.
Estimated eGFR (CKD-EPI) ≥45 mL/min.
If applicable, the last regular somatostatin analogue injection should be administered 2 weeks+/−1 week prior to SST PET scan for long-acting release forms.

### Exclusion Criteria

Known hypersensitivity to [⁶¹Cu]Cu, to NODAGA, to LM3 or to any of the excipients of [⁶¹Cu]Cu-NODAGA-LM3.
Prior or planned administration of a radiopharmaceutical within 8 half-lives of the radionuclide used on such radiopharmaceutical including at any time during the current study.
Initiation or continuation of active anti-tumor treatment between [⁶¹Cu]Cu-NODAGA-LM3 and [⁶⁸Ga]Ga-DOTATOC PET/CT, except continuation of long-acting somatostatin analogues.
Presence of active infection at screening or history of serious infection within the previous 6 weeks.
Pregnant or breast-feeding women.
History of somatic or psychiatric disease/condition that may interfere with the objectives and assessments of the study.

### 5.4.4. Measurements and Procedures

### Consent and Randomization

Collection of information regarding past medical/surgical history and anamnesis (especially regarding functional activity).
Collection of the medication list: if patients are treated with somatostatin analogues date of last injection should be noted and PET/CT appointment coordinated.
Collection of baseline safety clinical and laboratory parameters, including vital signs (blood pressure, temperature, heart rate, oxygen saturation), differential blood cell count, renal and liver function tests) pre-dosing of the IMP; These will be repeated ˜2-4 h post IMP-administration.
If woman in child-bearing age: B-HCG test will be performed.
Collection of baseline patient reported stress (stress thermometer); this will be repeated ˜1 h and ˜3 h post injection.

Intravenous administration of [⁶¹Cu]Cu-NODAGA-LM3 composition (Table 22 dose) (slow bolus).
Pharmacokinetics and blood-based dosimetry analysis: Blood will be collected at 0 (pre-dosing), 2, 5, 10, 20 and 30 min and at ˜1, ˜2, ˜4 and ˜18 h post [⁶¹Cu]Cu-NODAGA-LM3 administration to estimate time activity curve and blood clearance for pharmacokinetics (in 6 patients).

### Imaging Procedures & Interpretation

Whole-body [⁶¹Cu]Cu-NODAGA-LM3 PET/CT scans will be performed at the investigative site.
[⁶¹Cu]Cu-NODAGA-LM3 PET/CT scans will be acquired ˜1 h and ˜3 h post-injection (list-mode acquisition in 6 patients to determine optimal injected activity and signal-to-noise ratio).
Image Data are transferred from the site to ABX-CRO using the image transfer tool ABX-DIRECT. During the transfer the image data are de-identified and trial specific patient ID and patient name is set as entered by the center. Image data are de-identified by the client before uploading to the server.
If it is not possible for the centre to transfer image data using ABX-DIRECT an alternative transfer using ABX-CROs secure file transfer protocol (sFTP) server is possible. The data upload via sFTP does not automatically pseudonymise the patient image files; therefore, patient image files need to be properly pseudonymised by the centre operator before transfer.
Naming convention for patient data consist of Study ID followed by a three-digit consecutive patient ID, starting at 001. Thus, a typical patient image identification number will appear as ‘COPPER PET-001’.
All trial PET/CT imaging data are processed at the centre to generate the following types of reconstructed images applying standard quantitative corrections (prior or during reconstruction):
   Normalization
   Randoms correction
   Dead time correction
   Decay correction
   Attenuation correction
   Scatter correction
All PET/CT imaging data are reconstructed using a 3D OSEM+Time of Flight (TOF) reconstruction as qualified during the equipment setup.
For dosimetry: one extra low-dose and non-enhanced PET/CT scan will be acquired ˜18 h post [⁶¹Cu]Cu-NODAGA-LM3 injection.
For determining biodistribution and the most suitable time window for imaging, reconstructed [⁶¹Cu]Cu-NODAGA-LM3 PET/CT static images, acquired ˜1 and ˜3 h p.i, will be read in order to measure organ and lesion uptake (SUVmax), tumor detection rate and tumor-to-background ratios (TBR). Scans will be read head-to-head by one board-certified nuclear medicine physician who will not be involved in the diagnostic efficacy analysis for measuring:
   Normal organ distribution: regions of interest (ROI) will be drawn over the following organs, excluding focal lesions: pituitary gland, salivary glands, thyroid gland, lungs, liver, spleen, pancreas, small intestine, adrenal glands, kidney, urinary bladder, upper thoracic spine and 3rd to 5th lumbar vertebrae. SUVmax and SUVmean will be reported for all listed organs.
   TBR: the liver will be defined as background organ for lesion located in the liver; for all other lesions, an adjacent spherical ROI of 1 cm will be placed in the immediate vicinity of the lesion. SUVmax will be reported for lesions and SUVmax and SUVmean will be reported for background organs.
For performing the diagnostic efficacy analysis, reconstructed [⁶¹Cu]Cu-NODAGA-LM3 PET/CT ˜1 h and ˜3 h post-injection and [⁶⁸Ga]Ga-DOTA-TOC PET/CT images (1 h p.i) will be read by two independent readers. Readers will be blinded for patient history, clinical information and radiopharmaceutical as well as time-point. Reading will be performed according to a locked-read methodology: Once the reader reviewed the images, the lesion number, location, and uptake will be recorded in the case report form.
Findings reconciliation: after all measurements are finished, the readers will be permitted to reopen the two scans again and compare the data for reconciliation. However, any revision to the original data will not be allowed at this phase.
   Focal lesion: any focal accumulation of [⁶¹Cu]Cu-NODAGA-LM3 and [⁶⁸Ga]Ga-DOTA-TOC not explained by physiologic uptake or benign lesions, such as bone trauma, hemangioma, degeneration disease will be considered suspicious lesions.
   Concordant non-physiological foci of uptake visible on any of the three imaging modalities ([⁶¹Cu]Cu-NODAGA-LM3 PET/CT, [⁶⁸Ga]Ga-DOTA-TOC PET/CT and conventional imaging, whether CE-CT or MRI) will be considered true positive lesion.
   Discordant non physiological foci of uptake visible on PET/CT will be verified by biopsy whenever possible and indicated or by best imaging follow-up (MRI Abdomen and/or CE-CT CAP and/or [⁶⁸Ga]Ga-DOTATOC PET/CT and/or [¹⁸F]F-FDG PET/CT) 2-7 months after visit 1 during follow up. For liver metastases, a dedicated liver MRI (including sequences acquired after injection of liver specific contrast medium) will be used as the gold standard.
Sensitivity Analysis: For each scan, exported table of reader 1 and reader 2 will be merged and processed by a 3rd reader to clarify possible discrepancies and to determine lesion location (lymph node, liver, peritoneum, mesenterium, lung, bone other (e.g. breast, heart, subcutis, orbit, brain etc). For this, reader 3 will need to reopen side-by-side the session of reader 1 and 2 to identify discrepancies.
   Example of discrepant lesions:
      Lesions missed or not identified as suspicious by one reader but captured by the other reader.
      Lesions that are counted as 2 lesions by one readers but as 1 lesion by the other (see example below).
   Lesions identified as discrepant by the 3rd reader will be reconciliated during a new consensus reading with reader 1 and 2 and the assistance of the 3rd reader. At the end of this analysis following information will be collected:
      a) unique lesion identification (lesion ID number and Tumor location)
      b) total number of lesions per scan
      c) total number of true discrepant lesions between readers for a given scan
      d) total number of matched lesions between reader 1 and 2 for a given scan (by calculation: d=total number of lesions (b)−total number of discrepant lesions (c))
   The 3rd reader will conduct a head-to-head review of all 3 scans of a given patient and will determine differences between scans. Matching lesions that are identified on all 3 scans (lesion type 1) will be defined as “true positive” lesions. Mismatch lesions (lesion type 2-6) will need adjudication against standard of truth (Best follow-up imaging 2-7 months post administration of [⁶¹Cu]Cu-NODAGA-LM3 composition or biopsy) by a board certified radiologist (if best std of truth imaging for this patient is CT or MR) and by a nuclear medicine physician (if best std of truth imaging is PET/CT).
   This step will allow collection of the following information:
   a) total number of matched lesions between [⁶⁸Ga]Ga-DOTATOC, [⁶¹Cu]Cu-NODAGA-LM3 @1 h and [⁶¹Cu]Cu-NODAGA-LM3 @3 h PET/CT scans for reader 1 and for reader 2
   b) total number of mismatched lesions (Type 2 to 6 lesions) between [⁶⁸Ga]Ga-DOTATOC, [⁶¹Cu]Cu-NODAGA-LM3 @1 h and [⁶¹Cu]Cu-NODAGA-LM3 @3 h PET/CT scans for reader 1 and for reader 2
   c) true positives (TP), true negatives (TN), false positives) (FP), and false negatives (FN
   d) Sensitivity, Positive Predictive Value, Specificity and Diagnostic Accuracy will be calculated based on TP, TN, FP and FN.
For the image-based dosimetry analysis regions of interest will be drawn on normal organs by a board certified nuclear medicine physician who will not be involved in the diagnostic efficacy analysis. The following organ will be three-dimensionally segmented: known SST2-expressing organs or any organ with positive visual uptake (excluding tumor foci) on PET imaging: e.g., pituitary gland, salivary glands, thyroid gland, lung, liver, spleen, pancreas, small intestine, adrenal glands, kidney and urinary bladder). A blood-based bone marrow dosimetry will be performed. Dosimetry calculation will be performed by an experienced medical physicist.
For simulating different injected activities, list mode [⁶¹Cu]Cu-NODAGA-LM3 PET/CT data acquired 1 h post-injection will be reconstructed; analysis of signal-to-noise ratio (Mean count rates and standard deviation in defined organs and tumor lesions will be measured).
   Hard- and Software used for Reading of Images:

The dosimetry software QDOSE® will be used for organ VOI determination. Evaluation will be performed at dedicated reading workstations located at ABX-CRO. The display system used for image evaluation will be assessed according to DIN 6868-157 “Image quality assurance in diagnostic X-ray departments—Part 157: X-ray ordinance acceptance and constancy test of image display systems in their environment”. The reader will evaluate the data in a dedicated reading room. Alternatively, the reader can use Citrix VirtualApp to access the evaluation server remotely and perform the reading from a remote reading workstation with internet connection. The display system used for image evaluation will be assessed according to DIN 6868-157. The image data and evaluation results remain at the evaluation server hosted at ABX-CRO.

### Organ Measurement:

To calculate the healthy organs absorbed dose and thus assess the safety, the organs volumes need to be determined on the CT image data. Image Review Workflow for organ volume determination:

1. The following organs will be segmented: known SST2-expressing organs or any organ with positive visual uptake (excluding tumor foci) on PET imaging (up to 10 organs). For image based red marrow dosimetry, the lumbar vertebrae will be segmented.

2. Organ volume will be determined on the CT image data by segmenting the organ. Organs can be segmented using automated methods and will be adjusted manually. Alternatively, organs can be segmented by manually drawing a boundary around the organ in different slices of the imaging data set. The 2D boundaries will be used to automatically create a 3D boundary that includes the whole organ. The boundary can be edited manually. Within this boundary, semi-automatic or threshold-based algorithms can be used to segment the organ volume. The segmentation can be edited manually. Organ segmentation will be performed by a radiologist or a medical imaging specialist.

3. Organ volumes will be verified and recorded Organ Measurement Worksheet (OM-WS) in paper form or in an electronic form.

If no organ volume can be determined on the CT imaging data, the value for the organ volume will be documented as “Not Evaluable” (NE) on the OM-WS. Thereafter the standard mass for the organ for the adult male phantom (patients with total body mass equal to or exceeding 64.5 kg) or the 15-year-old phantom (patients with total body mass less than 64.5 kg) from the ICRP publication 89 will be used for absorbed dose calculation.

For dosimetry, whole body PET/CT scans will be acquired in a supine position after the ⁶¹Cu-NODAGA-LM3 injection according to the imaging schedule at Error! Reference source not found. will be used for safety dosimetry. The PET data are quantitatively reconstructed at the site using a 3D OSEM+TOF algorithm. The VOIs are copied from the CT image to the PET image and can be edited if needed. This VOI will define the anatomical volume of the organ. The VOI to collect the total activity of the organ will be defined by convolving the anatomical VOI with a Gaussian function using a suitable FWHM. The resulting total activity (in MBq) is obtained for each segmented VOI and imaging time point and saved for later processing. Organs which cannot be determined in the PET images will be documented as “Not Evaluable”.

### Safety Dosimetry:

The cumulated activities or residence times of organs and remainder body are used as input for safety dosimetry calculation using OLINDA/EXM 2.2 (Stabin M G et al., 2005). The organ mass will be determined from the segmentation on the CT and used for target organ mass adaption in OLINDA/EXM 2.2. Output OLINDA/EXM v2.2 are:
1. Equivalent dose (mSv) and normalized equivalent dose (mSv/MBq) for a number of target organs and the total body.
2. Whole body effective doses (mSv) and normalized effective doses (mSv/MBq) according to ICRP 103. The effective dose will be the mean effective dose of male and female phantom according to ICRP 103.

The organ doses reported are those calculated with OLINDA/EXM 2.2. The results are used to determine the organs receiving the highest organ dose and dose limiting organs. In case the dosimetry assessment needs to be reperformed a new dosimetry report will be exported.

### Standard of Care Procedures

[⁶⁸Ga]Ga-DOTA-TOC PET/CT including native and contrast-enhanced CT (performed for each patient within 4 weeks before or after of [⁶¹Cu]Cu-NODAGA-LM3 administration and on the same scanner).
MRI and/or CE-CT and/or [⁶⁸Ga]Ga-DOTATOC PET/CT and/or [¹⁸F]F-FDG PET/CT 2-7 months after [⁶¹Cu]Cu-NODAGA-LM3 PET/CT.
Contrast enhanced liver MRI will be performed for each patient with known or suspected liver metastases within 3 months of [⁶¹Cu]Cu-NODAGA-LM3 administration.

### 5.4.5. Statistical Considerations

### Statistical Methods:

Primarily, all variables will be analyzed descriptively, as follows: mean, standard deviation, median and range for continuous variables; median, range and frequency distribution for discrete (ordinal) variables; frequency distribution for nominal variables.

Secondarily, the variables and their probabilities (except for sensitivity) will be compared using non-parametric tests and interpreted in an exploratory manner. 95% confidence intervals will be estimated as appropriate.

For the primary endpoint, safety, no formal sample size calculation was performed (see above). All data will be analyzed by descriptive statistics methods, including frequency and severity of adverse events, abnormal findings in physical examination, vital signs and clinical laboratory parameters. The safety analyses will be conducted on the Safety Analysis Set, which consists of all enrolled patients who received study medication, regardless of any protocol deviations.

For the co-primary endpoint, sensitivity, the sample size was calculated to ensure sufficient power for showing both superior and non-inferior sensitivity of [⁶¹Cu]Cu-NODAGA-LM3 PET/CT over [⁶⁸Ga]Ga-DOTATOC PET/CT.

For the secondary endpoints: dosimetry, biodistribution and pharmacokinetic study (6 patients), no formal sample size is required. According to our previous experience with a similar radiotracer ([⁶⁸Ga]Ga-NODAGA-JR11) and owing to the limited variability in estimating total body mean effective dose (standard deviation ˜7-10%) we expect that 6 patients are sufficient to appropriately address this endpoint.

### GCP Statement

This study will be conducted in compliance with the protocol, the current version of the Declaration of Helsinki, the ICH-GCP or ISO EN 14155 (as far as applicable) as well as all national legal and regulatory requirements.

### 5.4.6. Imaging Results

Six subjects meeting the following inclusion criteria participated in the human clinical studies as described herein in Example 4 and the study flowchart can be found in FIG. 11.

Inclusion criteria: 1) Well-differentiated BC-NET or GEP-NET (incl. NET G3 with Ki-67 <50%). 2) Clinical indication to ⁶⁸Ga-DOTATOC PET/CT. 3) At least 2 lesions detected on previous ⁶⁸Ga-DOTATOC PET/CT: 5 patients ⁶⁸Ga-DOTATOC PET/CT negative.

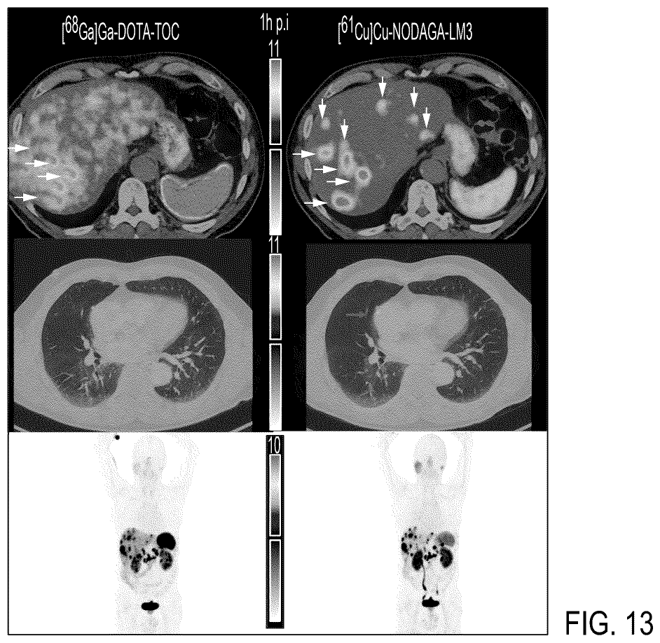

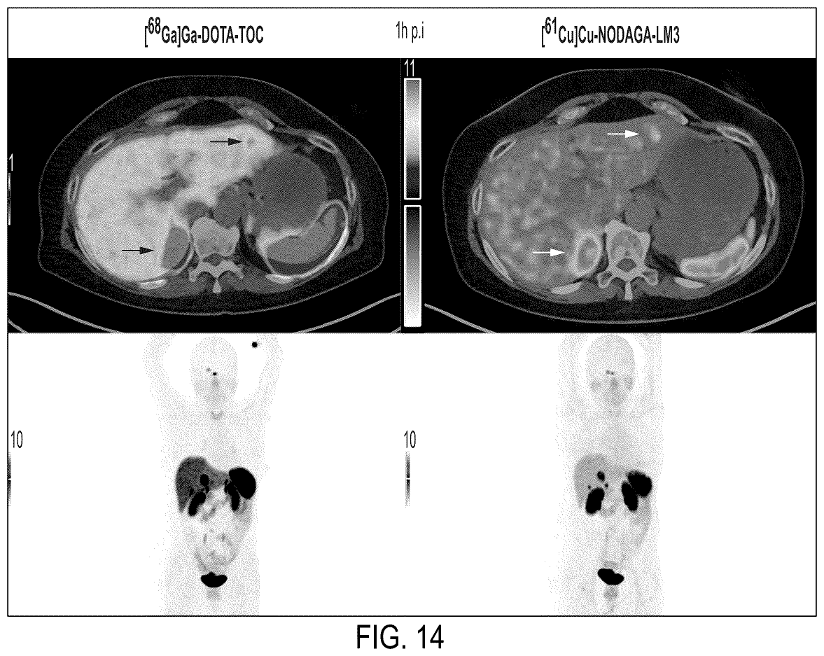

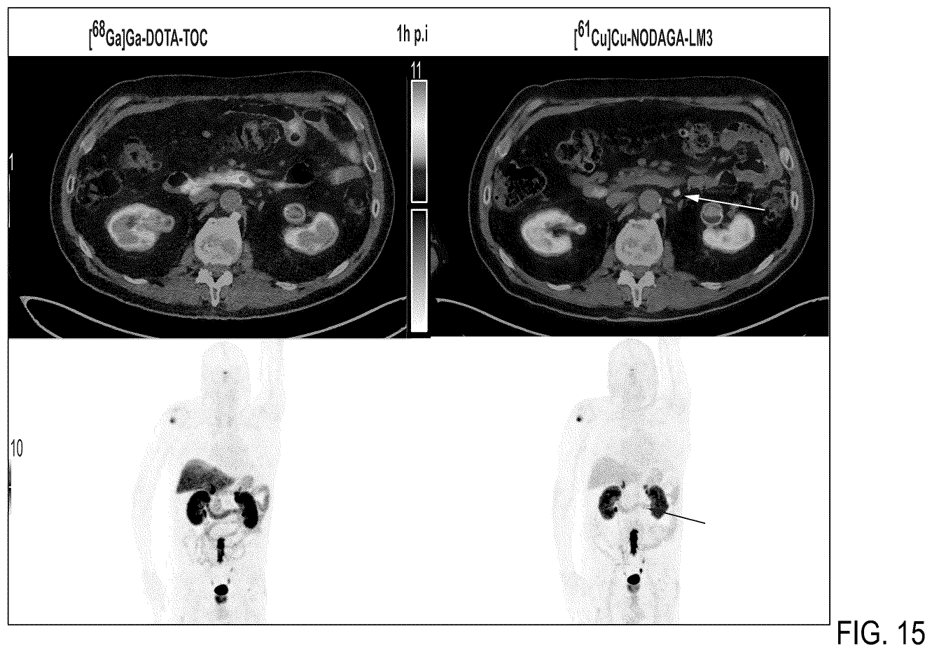

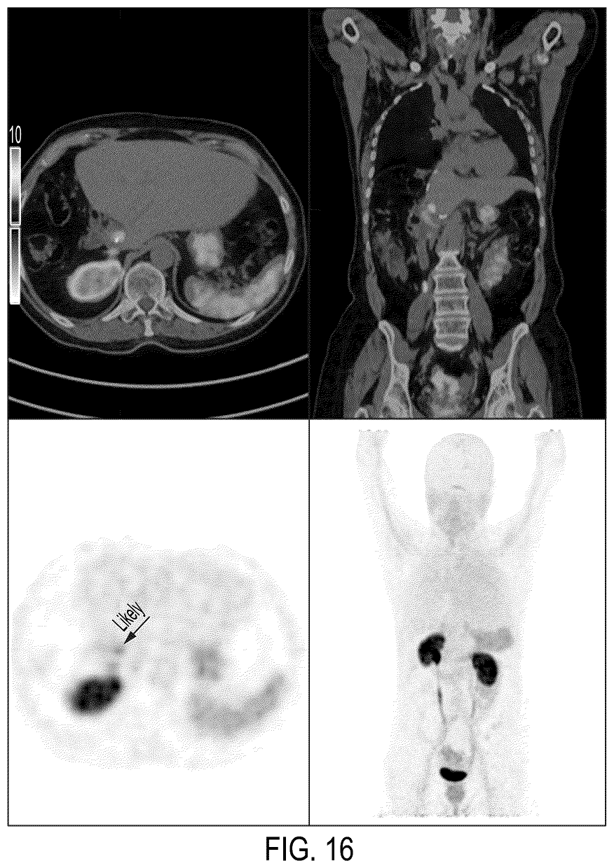

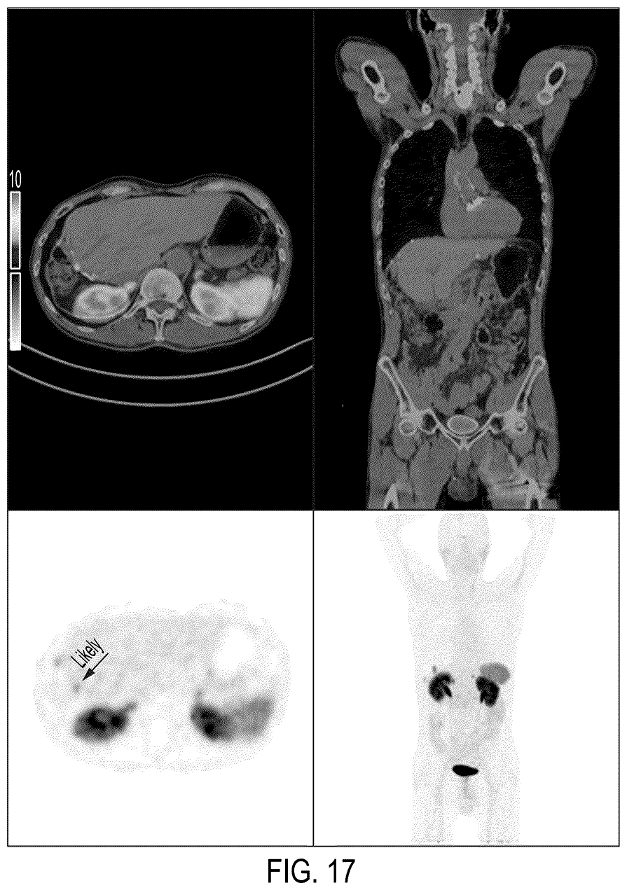

PET/CT imaging results of Subjects 1˜4 and 6 demonstrated that [⁶¹Cu]Cu-NODAGA-LM3 has the advantage of visualizing more lesions, including small and new lesions, than ⁶⁸Ga-DOTATOC. [⁶¹Cu]Cu-NODAGA-LM3 also has higher tumor uptake and very low physiological uptake in the liver, spleen, and GI tract, resulting in better tumor-to-background ratio.

[⁶¹Cu]Cu-NODAGA-LM3 PET/CT and [⁶⁸Ga]Ga-DOTATOC PET/CT of study participants are reported in FIGS. 13-17.

### 5.5. Example 5: Exemplary Method of Measuring Apparent Molar Activity

A [⁶¹Cu]CuCl₂ solution may be characterized by apparent molar activity (AMA). Apparent molar activity assesses, for example, competing trace metal impurities of ⁶¹Cu chelation by quantifying the minimum amount of a given chelator required for efficient radiolabeling.

An AMA test measures the percentage of complexation, by radio-TLC, after titrating a quantity of [⁶¹Cu]CuCl₂ solution with different amounts of a chelator (e.g., DOTA, NODAGA, etc).

The results were then plotted (x-axis (logarithmic): nmol of chelator; y-axis: % of complexation). The lowest nmol value of chelator corresponding to ≥95% complexation achieved was recorded.

The experimental AMA value was then calculated according to the formula:

$$AMA value ( experimental ) = \frac{Activity [ MBq ]}{{n}_{NODAGA} [ nmol ]}$$

where:
   Activity: indicates the activity present in the fixed amount of [⁶¹Cu]CuCl₂ solution used for the titration decay corrected at the EoP (end of production; end of bombardment plus 1 hour).
   n_{NODAGA}: indicates the lowest nmol value of chelator for which ≥95% complexation was achieved.

This test was performed to assess, for example:
1) the grade of a chemical employed for the manufacturing of the [⁶¹Cu]CuCl₂ solution;
2) the quality of a consumable employed for the manufacturing of the [⁶¹Cu]CuCl₂ solution;
3) the target coin manufacturing process; or
4) in case poor radiolabeling yields were observed.

### AMA Test Procedure

The mobile phase was prepared as (0.1M sodium citrate) as follows:
   Weigh 5.882 g of trisodium citrate dihydrate.
   Add Suprapur water up to a volume of 200 mL.
   Adjust the pH by adding HCl dropwise until it reaches a pH of 5.
Transfer an adequate amount of mobile phase into the TLC development chamber to cover a depth of 5 mm.
Prepare the sodium acetate solution (0.5 M in Ultrapur water) as follows:
   Weigh 2.051 g of sodium acetate.
   Add Ultrapur water up to a volume of 50 mL.
   Apply 10 μL of the solution on a pH strip and check if the pH was 8.
A 5 mM stock solution of NODAGA was prepared in 0.5 M sodium acetate (Stock 1) as follows:
   Weigh 3.1 mg of NODAGA.
   Add Ultrapur water or equivalent up to a volume of 1.5 mL.
   Store at −20° C., after use.
A 50 μM stock solution of NODAGA was prepared in 0.5 M sodium acetate (Stock 2) by mixing 10 μL of Stock 1 with 990 μL 0.5 M sodium acetate. Store at −20° C., after use.
NODAGA test solutions S1-S2 were prepared for chelator titration as displayed in Table T1 by diluting Stock 2 with the listed volumes of sodium acetate 0.5 M. The test solution S0 was a blank solution (without the addition of NODAGA) of 100 μL of 0.5 M sodium acetate.

TABLE T1
Preparation of solution 1-3 for chelator titration.

| Solution | c_{NODAGA} | Volume | Volume sodium |
|---|---|---|---|
| name | [nM] | stock 2 [μL] | acetate [μL] |
| S0 | 0 | 0 | 100 |
| S 1 | 3000 | 100 | 1567 |
| S 2 | 1500 | 50 | 1617 |
| S 3 | 900 | 30 | 1637 |

The test solutions, S4-S10, 1:10 dilutions of the S1-S7 were prepared with 0.5 M sodium acetate according to the dilution scheme shown in Table.

TABLE T2
Preparation of solution 4-10 for chelator titration.

| Solution | c_{NODAGA} | Volume | Volume sodium |
|---|---|---|---|
| name | [nM] | Sx [μL] | acetate [μL] |
| S 4 | 300 | 100 of S1 | 900 |
| S 5 | 150 | 100 of S2 | 900 |
| S 6 | 90 | 100 of S3 | 900 |
| S 7 | 30 | 100 of S4 | 900 |
| S 8 | 15 | 100 of S5 | 900 |
| S 9 | 9 | 100 of S6 | 900 |
| S 10 | 3 | 100 of S7 | 900 |

A solution of 0.05 M HCl was prepared as follows:
   Add 5 mL of Ultrapur water in a 15 mL Falcon tube.
   Add 53 μL of 30% HCl to the falcon using a micropipette.
   Add Ultrapur water up to 10 mL.
   Spot 10 μL of the solution on a pH strip and confirm the pH is between 1.0 and 1.6.
The [⁶¹Cu]CuCl₂ solution was diluted with 0.05 M hydrochloric acid to reach a total volume of 1 mL with an activity concentration of 0.2 MBq/μL (EoP) according to the following formula:

$${[}^{61} Cu ] {CuCl}_{2} solution [ μL ] = \frac{0.2 [ MBq / μL ]}{A C ( EoP ) [ MBq / μL ]} * 1000 μL$$

0.05 M hydrochloric acid for dilution=1000 μL-[⁶¹Cu]CuCl₂ solution to draw [μL] where AC (EoP) is the activity concentration at EoP.
   The solution were named “diluted Cu-61 solution”. See the example in Table.

TABLE T3
[⁶¹Cu]CuCl₂ dilution examples.

| Activity | | Activity | |
|---|---|---|---|
| eluted at | | concentration | |
| EoP | Volume | at EoP | Dilution for AMA |
| [MBq] | [mL] | [MBq/μL] | test |
| 1000 | 3.0 | 0.33 | 600 μL of [⁶¹Cu]CuCl₂ |
| | | | diluted with 400 μL of |
| | | | 0.05M HCl |
| 2000 | 3.0 | 0.67 | 300 μL of [⁶¹Cu]CuCl₂ |
| | | | diluted with 700 μL of |
| | | | 0.05M HCl |
| 3500 | 3.0 | 1.17 | 233 μL of [⁶¹Cu]CuCl₂ |
| | | | diluted with 767 μL of |
| | | | 0.05M HCl |

Add in each solution for chelator titration 50 μL of diluted [⁶¹Cu]CuCl₂ solution (as prepared in Table T3), obtaining the reaction solutions listed in Table.

TABLE T4
Reaction solutions.

| | | Volume of | Volume | | | |
|---|---|---|---|---|---|---|
| Reaction | | NODAGA | of ⁶¹Cu | Finale | | |
| solution | NODAGA | solution Sx | solution | volume | n_{NODAGA} | c_{NODAGA} |
| name | solution | [μL] | [μL] | [μL] | [nmol] | [nM] |
| RS 1 | S1 | 100 | 50 | 150 | 0.3 | 2000 |
| RS 2 | S2 | 100 | 50 | 150 | 0.15 | 1000 |
| RS 3 | S3 | 100 | 50 | 150 | 0.09 | 600 |
| RS 4 | S4 | 100 | 50 | 150 | 0.03 | 200 |
| RS 5 | S5 | 100 | 50 | 150 | 0.015 | 100 |
| RS 6 | S6 | 100 | 50 | 150 | 0.009 | 60 |
| RS 7 | S7 | 100 | 50 | 150 | 0.003 | 20 |
| RS 8 | S8 | 100 | 50 | 150 | 0.0015 | 10 |
| RS 9 | S9 | 100 | 50 | 150 | 0.0009 | 6 |
| RS 10 | S10 | 100 | 50 | 150 | 0.0003 | 2 |
| RS 0 | S0 | 100 (Only 100 | 50 | 150 | 0 | 0 |
| | | μL of sodium | | | | |
| | | acetate 0.5M) | | | | |

The reaction solutions were incubated at room temperature for 5 minutes.
After incubation, perform TLC measurement on all the reaction solutions as follows:
   Using a micropipette, spot 2 μL of each [⁶¹Cu]Cu-NODAGA reaction solution RSO-RS10 onto different pre-cut TLC paper.
   Allow the TLC paper to dry for about 5 minutes or until there was no visible stain.
   Place the TLC paper in the TLC chamber with the stained side on the bottom of the chamber. When the solvent front was about 1 cm from the top of the strip, remove the TLC paper from the TLC chamber with tweezers.
   Mark the solvent front on the TLC paper strip with a pencil.
   Measure the distance between the starting line and the solvent front with a ruler and let the strip dry completely.
After scanning the TLC plate, the software (e.g., Biochrom) will generate an evaluation report. Print the evaluation report and record the results. All data (i.e., RF, % of total activity, % of ROI) of the peaks must be documented.
Using the regions of interest (ROI) technique (drawing regions over distinct areas of activities), the % of complexation of [⁶¹Cu]Cu-NODAGA was expressed as a percentage of the total detected activity, as follows:

$${Complexation}_{[ 61 C u ] C u - N O D A G A} ( \% ) = \frac{{Activity}_{{[}^{61} C u ] C u - N O D A G A} [ counts ]}{Total activity [ counts ]} * 100$$

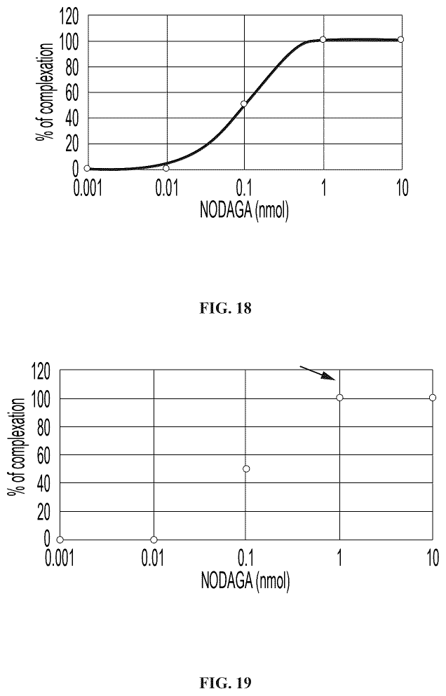

The TLC scanner results were plotted (x-axis (logarithmic): nmol of NODAGA chelator; y-axis: % of complexation), and will follow a sigmoidal trend. An example of plot was given in FIG. 18.
The lowest value of nmol of chelator in correspondence of which ≥95% complexation is identified on the plot. An example was reported in FIG. 19.

The experimental AMA value was then calculated according to the formula:

$$AMA value ( experimental ) = \frac{Activity [ MBq ]}{{n}_{NODAGA} [ nmol ]}$$

where:
   Activity: indicates the activity present in the fixed amount of [⁶¹Cu]CuCl₂ solution used for the titration decay corrected at the EoP.
   n_{NODAGA}: indicates the lowest value of nmol of chelator where ≥95% complexation was achieved.

### 6. EQUIVALENTS AND INCORPORATION BY REFERENCE

While aspects of this disclosure have been particularly shown and described with reference to a preferred embodiment and various alternate embodiments, it will be understood by persons skilled in the relevant art that various changes in form and details can be made therein without departing from the scope of the disclosure.

All references, issued patents, and patent applications cited within the body of the instant specification are hereby incorporated by reference in their entirety, for all purposes. In particular, U.S. Provisional Patent Application No. 63/568,814 (filed on Mar. 22, 2024) and International Application Nos. PCT/US2023/75064 (filed Sep. 25, 2023), PCT/US2023/75067 (filed Sep. 25, 2023), and PCT/US2025/21199 (filed Mar. 24, 2025) are hereby incorporated by reference in their entirety.

1. A method for imaging a subject comprising:
(a) administering an effective amount of a pharmaceutical composition to a subject suspected of having or diagnosed with a somatostatin receptor subtype 2 (SST2)-expressing tumor, wherein the pharmaceutical composition comprises:
i. a radiotracer that has the structure 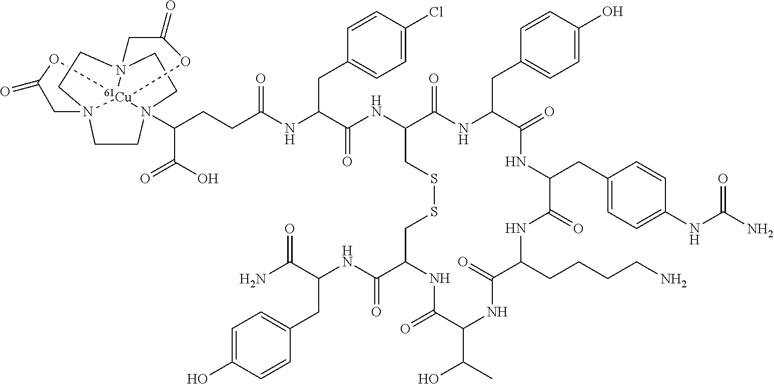
or is a pharmaceutically acceptable salt thereof; and
ii. a radiolytic inhibitor;
wherein the pharmaceutical composition is characterized by one or more of:
a [⁶¹Cu]Cu radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a ¹⁰⁹Cd specific activity≤0.1 Bq/g; and
(b) generating one or more radiographic images of the subject.

2. (canceled)

3. A method for determining a subject's response to a cancer treatment comprising:
(a) administering an effective amount of a pharmaceutical composition to a subject diagnosed with a SST2-expressing tumor at an earlier time point and at a later time point, wherein the pharmaceutical composition comprises:
i. a radiotracer that has the structure 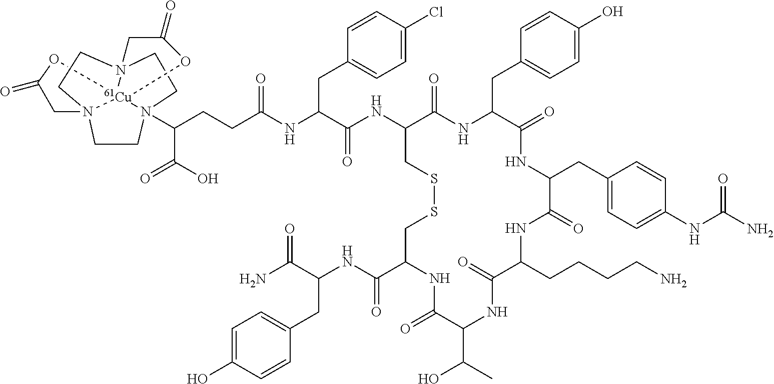
or is a pharmaceutically acceptable salt thereof; and
ii. a radiolytic inhibitor;
wherein the pharmaceutical composition is characterized by one or more of:
a [⁶¹Cu]Cu radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a ¹⁰⁹Cd specific activity≤0.1 Bq/g;
(b) generating one or more radiographic images of the subject at the earlier time point and at the later time point;
(c) determining the amount of localization of the radionuclide in the one or more radiographic images of the subject at the earlier time point and at the later time point; and
(d) determining the subject's response to the cancer treatment by comparing the amount of localization of the radionuclide in the one or more radiographic images of the subject at the earlier time point and at the later time point.

4-81. (canceled)

82. A pharmaceutical composition comprising:
(a) a radiotracer that has the structure 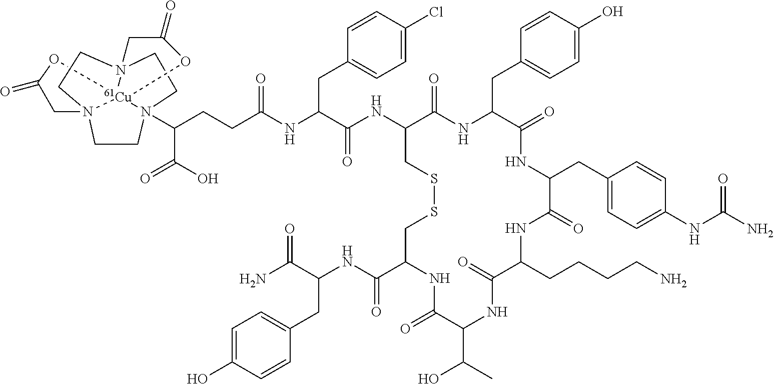
or is a pharmaceutically acceptable salt thereof,
(b) a radiolytic inhibitor, and
(c) an aqueous vehicle;
wherein the pharmaceutical composition is characterized by one or more of:
a [⁶¹Cu]Cu radionuclidic purity at end of synthesis of ≥97%,
a radiocobalt activity content at end of synthesis of ≤0.05%,
a ^{110m}Ag specific activity≤0.1 Bq/g,
a ^{108m}Ag specific activity≤0.1 Bq/g, or
a ¹⁰⁹Cd specific activity≤0.1 Bq/g.

83. The pharmaceutical composition according to claim 82, wherein the activity of the pharmaceutical composition is ≥25 MBq.

84. (canceled)

85. The pharmaceutical composition according to claim 82, wherein the radiotracer radiochemical purity of the pharmaceutical composition is ≥90% at 12 hours after end of synthesis.

86. (canceled)

87. The pharmaceutical composition according to claim 82, wherein the pharmaceutical composition is characterized by a ⁶¹[Cu]Cu radionuclidic purity of the pharmaceutical composition at end of synthesis of ≥98%.

88. The pharmaceutical composition according to claim 82, wherein the pharmaceutical composition is characterized by a radiocobalt activity content at end of synthesis of ≤0.01%.

89. The pharmaceutical composition according to claim 82, wherein the pharmaceutical composition is characterized by at least one of a ⁵⁶[Co]Co specific activity or ⁵⁸[Co]Co specific activity of ≤1,500 Bq/g.

90-92. (canceled)

93. The pharmaceutical composition according to claim 82, wherein the pharmaceutical composition is characterized by one or more of: a ^{110m}Ag specific activity≤0.1 Bq/g, a ^{108m}Ag specific activity≤0.1 Bq/g, or a ¹⁰⁹Cd specific activity≤0.1 Bq/g.

94. The pharmaceutical composition according to claim 82, wherein the sum of the specific activities of the radionuclidic impurities in the pharmaceutical composition is ≤8,000 Bq/g.

95. The pharmaceutical composition according to claim 82, wherein the pharmaceutical composition is characterized by one or more of: Al≤1.2 ng/MBq, Co≤0.2 ng/MBq, Fe≤1.7 ng/MBq, Pb≤0.8 ng/MBq, or Zn≤0.8 ng/MBq.

96. The pharmaceutical composition according to claim 82, wherein the activity concentration of the pharmaceutical composition is ≥10 MBq/mL.

97. (canceled)

98. The pharmaceutical composition according to claim 82, wherein the apparent molar activity of the radiotracer is ≥1 MBq/nmol.

99. The pharmaceutical composition according to claim 82, wherein the radiotracer is present in an amount ≥1 μg.

100. (canceled)

101. The pharmaceutical composition according to claim 82, wherein the aqueous vehicle comprises isotonic saline.

102. The pharmaceutical composition according to claim 82, wherein the radiolytic inhibitor is present in the pharmaceutical composition in a concentration of ≥1 mg/mL.

103. The pharmaceutical composition according to claim 102, wherein the radiolytic inhibitor is present in the pharmaceutical composition in a concentration from 1 mg/mL to 10 mg/mL.

104. The pharmaceutical composition according to claim 82, wherein the radiolytic inhibitor is selected from ascorbic acid, gentisic acid, citric acid, N-tert-butyl-α-phenylnitrone (PBN), polyvinylpyrrolidone (PVP), ethanol, DMSA, cysteine, vanillin, methionine, adenine, dobesilic acid, thymine, uracil, nicotinic acid, nicotinamide, salts of any of the foregoing, and combinations thereof.

105. The pharmaceutical composition according to claim 104, wherein the radiolytic inhibiter is ascorbic acid or a salt thereof.

106. The pharmaceutical composition according to claim 82, further comprising sodium chloride, for example in a concentration from 0.5 mg/mL to 50 mg/mL.

107. The pharmaceutical composition according to claim 82, further comprising ethanol in an amount ≤10% v/v.

108. The pharmaceutical composition according to claim 82, comprising:
1-100 μg of the radiotracer,
5-20 mg of ascorbic acid or a salt thereof,
0.1-1 mL ethanol, and
1-10 mL isotonic saline solution.

109-110. (canceled)

111. The pharmaceutical composition according to claim 82, further comprising sodium bicarbonate, for example in an amount from 0.5 mg to 10 mg.

112. The pharmaceutical composition according to claim 82, further comprising a metal scavenger in an amount from 0.001 mg to 0.01 mg.

113. The pharmaceutical composition according to claim 82, further comprising propylene glycol in an amount from 5 mg to 100 mg.

114. The pharmaceutical composition according to claim 82, wherein the composition is formulated for intravenous infusion.

115. The pharmaceutical composition of claim 82, wherein the total volume of the pharmaceutical composition is from 2 mL to 15 mL.

116. The pharmaceutical composition according to claim 82, having ≥99% radiochemical purity at 3 hours after end of synthesis.

117. The pharmaceutical composition according to claim 82, wherein the pH of the pharmaceutical composition is from 5 to 7.

118. The pharmaceutical composition according to claim 82, wherein the radiotracer has the structure: 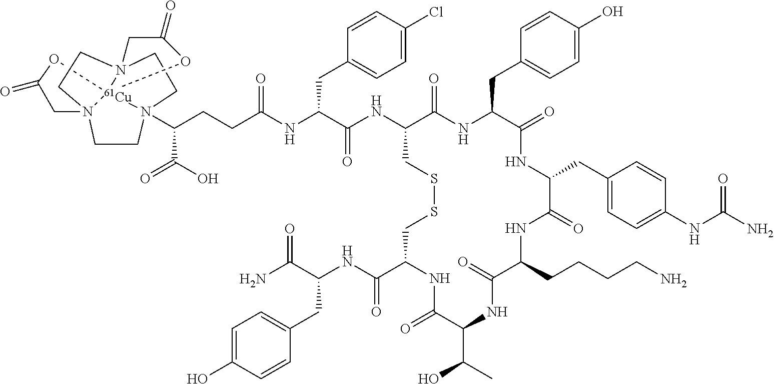
or is a pharmaceutically acceptable salt thereof.
